(19) United States
(12) Patent Application Publication
(10) Pub. No.: US 2023/0293798 A1
(43) Pub. Date: Sep. 21, 2023

(54) DEVICE FOR REMOVING UNDESIRED MATTER, PATHOGENS, AND TOXINS FROM A FLUID AND HUMAN BLOOD

(71) Applicant: Robert M. Lyden, Ashland, OR (US)

(72) Inventor: Robert M. Lyden, Ashland, OR (US)

(21) Appl. No.: 18/182,647

(22) Filed: Mar. 13, 2023

Related U.S. Application Data
(60) Provisional application No. 63/319,889, filed on Mar. 15, 2022.

(51) Int. Cl. A61M 1/36; A61M 1/14

(52) U.S. Cl. CPC A61M 1/3618; A61M 1/15632

(57) ABSTRACT

The present disclosure relates to a device for removing undesired matter, pathogens, and toxins from a fluid and human blood.

### CROSS REFERENCE TO RELATED APPLICATION

This application claims the benefit to U.S. Provisional Application Ser. No. 63/319,889, filed Mar. 15, 2022. The contents of the application cited in the CROSS REFERENCE TO RELATED APPLICATION is hereby incorporated by reference herein in its entirety.

### FIELD

The present disclosure relates to a device for removing undesired matter, pathogens, and toxins from a fluid and human blood.

### BACKGROUND

During the last four years the world's population has suffered a pandemic due to the COVID-19 virus. The virus continues to mutate and has now become an endemic which will persist in the future. In view of the evidence which has been presented in U.S. Congress by Representative Jim Jordan of Ohio which is shown in the following video and website: https://fb.watch/9g_ofuA2Px/, and by Senator Rand Paul of Kentucky which is shown of the following video and website, https://tb.watch/amQPQ4GQEf/, and elsewhere, e.g., see the article: “Military Documents About Gain of Function Contradict Fauci Testimony Under Oath,” Jan. 10, 2022 on the following website: https://www.projectveritas.con/news/nilitary-docunents-about-gain-of-function-contradict-fauci-testimony-under/, and in particular, Representative Paul Gosar's post on Facebook: https://www.facebook.com/repgosar/videos/459786482262168/, it appears that the COVID-19 virus was man-made and that Dr. Fauci, the National Institute of Health (NIH), and the company Ecohealth Alliance, were aware of this information in early 2020. The NIH has now admitted that it funded the gain of function research of Ecohealth Alliance and the Wuhan Lab's work in developing the COVID-19 virus, as shown in the following article and website: https://www.nationalreview.com/news/nih-admits-to-funding-gain-of-function-research-in-wuhan-says-ecohealth-violated-reporting-requirements/?fb_news_token=u6BFVGavP%2FPmFdJC7splew%3D%3D.FvLpMe6GY BCBOk%2F%2BgaiyYg9w4ReglwrvdbkGcmlaaitzcQbKJ%2BGHpnqKf%2Bna%2BZpsO3BZ5ALe2W36DA7OkRlBxM2UIfnbiiDYpAK81YL3AdcUcZ%2FOs6rcrqxZRe 40Xoi45xbRrWLSwh7OIPMxuctbiW7ekr%2FwwfAwbsKQUDbxywvGpiF%2FW8vhv Qi7V2eJO4BROYXOTYvN6q%2BrXYUOanE7RfEM1RoOvrFrCMk5jQEq8fwMBJ2bv 6Sj0TbQ5hf4qJd4TsleNZb3dMGbLEhVsNUcOo%2BsRPt10ZmpDDeA24qYM%2BcY JBk6sNEbu2fEGozI4NL5msE%2FP7hsL9jFIiGeCs%2F16IPU8GiwM%2FBJMIGBIs Gvs1yRiWOm%2B3%2BZ0D016oapEzPkNXox3zWY2nVfGUmeF6PO%3D%3D.

The development of pathogens which can constitute biological weapons is possibly in violation of both U.S. and International Law. See the Biological Weapons Convention which prohibits the development and production of biological and toxic weapons, the Nuremberg Code of ethics relating to medical research, and also the Nuremberg Principle VI relating to “crimes against humanity.” In this regard, see the information presented in the video: “David Martin Presents Hard Evidence Showing COVID-19 Is A Man-Made Bioweapon.” https://z3news.com/w/david-martin-presents-evidence-corona-virus-manmade/. Attorney Robert F. Kennedy, Jr., is the author of a relevant 480 page book entitled: “The Real Anthony Fauci, Bill Gates, Big Pharma, and the Global War on Democracy and Public Health,” published by Simon and Schuster, which is a national best seller and available as an e-book through Google, Amazon, and Kindle, e.g., see the website: https://anazon.com/Real-Anthony-Fauci-Democracy-Childrens/dp/1510766804.

In response to the COVID-19 pandemic, members of the executive and legislative branches of the United States Government, Dr. Fauci, the Center for Disease Control, the NIH, and companies in the pharmaceutical industry including Pfizer, Inc., Johnson & Johnson and their subsidiary Janssen Pharmaceuticals, and Moderna quickly developed so-called “vaccines.” However, the so-called “vaccines” which have been made by Pfizer and Moderna are not vaccines as most people familiar with the normal meaning of the word vaccine over the past 60 years would understand. Their so-called “vaccines” are forms of experimental synthetic gene therapy and are intended to reduce the severity of symptoms experienced when an individual becomes ill with the COVID-19 virus, but these so-called “vaccines” will not prevent individuals from getting ill or spreading the COVID-19 virus. A representative of Pfizer who testified before the European Union has admitted that their so-called “vaccine” was never even tested for this purpose, e.g., see https://youtu.be/vsbTeJOEMg0. In this regard, it is well known that many people who have been “vaccinated” have nevertheless become sick with COVID-19, and also communicated the virus to others including members of their own family. In support, see the following article: “Israel, One of the Most Vaccinated Countries in the World, Sets New COVID-19 Case Record:” https://nb.ntd.con/israel-one-of-the-most-vaccinated-countries-in-the-world-sets-new-covid-19-case-record_730957.htmlp.

The following link to the CDC website shows how it defined vaccines until late 2021 which provides the following definitions with the differences shown in red font below: https://www.ede.gov/vaccines/vac-gen/inz-basics.htm.

CDC Definitions of Vaccine and Vaccination 2015-2021:
Immunity: Protection from an infectious disease. If you are immune to a disease, you can be exposed to it without becoming infected.
Vaccine: A product that stimulates a person's immune system to produce immunity to a specific disease, protecting the person from that disease. Vaccines are usually administered through needle injections, but some can be administered by mouth or sprayed into the nose.
Vaccination: The act of introducing a vaccine into the body to produce immunity to a specific disease.
Immunization: A process by which a person becomes protected against a disease through vaccination. This term is often used interchangeably with vaccination or inoculation.

An article entitled “The CDC Suddenly Changes the Definition of ‘Vaccine’ and ‘Vaccination’ discloses the CDC's recent changes to the definition of vaccine made on Sep. 1, 2021 which are shown in below: https://www.citizensjournal.us/the-cdc-suddenly-changes-the-definition-of-vaccine-and-vaccination/.

CDC Definitions of Vaccine and Vaccination Sep. 1, 2021-Present:
Immunity: Protection from an infectious disease. If you are immune to a disease, you can be exposed to it without becoming infected.
Vaccine: A preparation that is used to stimulate the body's immune response against diseases. Vaccines are usually administered through needle injections, but some can be administered by mouth or sprayed into the nose.
Vaccination: The act of introducing a vaccine into the body to produce protection from a specific disease.
Immunization: A process by which a person becomes protected against a disease through vaccination. This term is often used interchangeably with vaccination or inoculation.

CDC Definition of Vaccination Pre-2015:
Vaccination: Injection of a killed or weakened infectious organism in order to prevent the disease.

Unfortunately, the so-called “vaccines” for COVID-19 are experimental, and have not gone through the same rigorous and lengthy research and approval process that was conducted by the FDA, e.g., in the development of the polio vaccine. In this regard, some of the pharmaceutical companies have also been less than forthright about the testing of their so-called “vaccines,” as disclosed in this video entitled “Bombshell News: Pfizer Lied To You About Their Vaccine!”: https://www.facebook.com/realCharlieKirk/videos/415015263369913/. Pfizer wished to keep all of its documents relating to the research and development of their “vaccine” sealed for 75 years, but a Court has ordered their release and the following website provides their document listing adverse reactions: https://pbmnpt.org/wp-content/uploads/2021/11/5,3,6-postmarketing-experience.pdf. In summary, the so-called “vaccines” are not in reality vaccines as that word has been used and understood over the past 60 years, but the pharmaceutical companies have used the word which then created a false impression and illusion in the public mind that they were protected from becoming infected with and also incapable of spreading COVID-19.

The use of the word vaccine may also create an impression in the public mind that the pharmaceutical companies are indemnified by the U.S. Government and legally protected from liability for any possible injuries caused by their so-called “vaccine” products. On information and belief, the pharmaceutical industry contributes more money to members of congress than any other lobby in our nation. In 1986, members of the pharmaceutical industry lobbied to get the National Childhood Vaccine Injury Act passed which indemnifies and protects the pharmaceutical companies from being sued by members of the public for their products possibly causing vaccine related injuries. Instead, there is a little-known government program which is supposed to provide benefits to people who can prove they have been seriously injured by a vaccine, but this program rarely pays and has covered less than 50 claims over the last decade. Accordingly, whereas only two or three vaccines were typically administered to children in the United States during 1950-1960's, the number of scheduled vaccines has now increased to over 30, and in part, because the pharmaceutical companies can hide behind the National Childhood Vaccine Injury Act of 1986 with regards to the harm caused by vaccine injuries. For more information, see the webpage for the National Childhood Vaccine Injury Act of 1986: https://www.congress.gov/bill/99th-congress/house-bill/5546, and also the news video: “You can't sue Pfizer or Modema if you have severe Covid vaccine side effects. The government likely won't compensate you for damages either” by CNBC, Dec. 17, 2020: https://www.cnbc.com/2020/12/16/covid-vaccine-side-effects-compensation-lawsuit.html, and also see the recent newspaper article entitled: “Vaccine Reaction Claims Stalled,” by Jeremy Olson, Minneapolis Star and Tribune, Sunday, Feb. 12, 2023.

In February, 2021, Health and Human Services Secretary Alex Azar invoked the Public Readiness and Emergency Preparedness (PREP) Act of 2005 which empowered him to provide legal protection to companies making or distributing critical medical supplies such as the so-called COVID-19 “vaccines” and treatments unless there is “willful misconduct” by the company and this protection will last until 2024. For more information, see the webpage for the PREP ACT of 2005: https://aspr.hhs.gov/legal/PREPact/Pages/default.aspx. According to Robert F. Kennedy, Jr., who is an attorney familiar with the relevant body of law, one of the reasons why Dr. Fauci and certain pharmaceutical companies have pushed for having children vaccinated has to do with those companies wanting to thereby obtain a legal shield from possible future litigation.

Within the scientific and medical community, there are now growing concerns about the nature and contents of the so-called COVID-19 “vaccines.” In this regard, several different researchers have discovered the presence of one or more substances and/or forms of matter in the so-called “vaccines” which have not been listed as ingredients or otherwise revealed to members of the public by the pharmaceutical companies. At least one of these substances and/or forms of matter has been identified as being a form of graphene oxide or graphene hydroxide. It is possible that these substances have been included as carriers and/or adjuvants in the so-called “vaccines,” e.g., see the definition of an immunologic adjuvant which is provided on the following website: https://en.m.wikipedia.org/wiki/immunologic adjuvant.

The following news sources, articles, and websites provide information relating to graphene substances: “Graphene Oxide: Introduction And Market News:” https://www.graphene-info.com/graphene-oxide?amp; “Graphene Oxide: A Carrier For Pharmaceuticals And A Scaffold For Cell Interactions:” https://pubmed.ncbi.nlm.nih.gov/25579346; “Magnetic Graphene Oxide: Synthesis Approaches, Physiochemical Characteristics, And Biomedical Applications:” https://www.sciencedirect.com/science/article/pii/S0165993621000133; “Toxicity of Graphene-Family Nanoparticles: A General Review Of The Origins And Mechanisms:” https://particleandfibrctoxicology.biomedcentral.com/articles/10.1186/s12989-016-0168-v; “Blood exposure to graphene oxide may cause anaphylactic death in non-human primates”: https://www.sciencedirect.conscience/article/pii/S1748013220300918; “Safety Assessment of Graphene-Based Materials: Focus on Human Health and the Environment:” https://pubmed.ncbi.nlin.nih.gov/30387986/. The use of some form of graphene oxide in the “vaccines” can be considered highly experimental. There are some indications that it can be associated with health risks, and the long-term effects of its presence in the human body are unknown.

In this regard, the aforementioned graphene substances can be electrically conductive and responsive to magnetic fields. The following article shows the “Radio-Frequency Characteristics of Graphene Oxide:” https://www.researchgate.net/publication/234845171_Radiofrequency characteristics of graphene oxide. The following link and its included videos show magnets sticking to the arms of different individuals at the site where they received their so-called “vaccine” shots: https://www.notonthebeeb.co.uk/magnetism.

In the following interview and video with Dr. Zandre' Botha, the blood of a patient is shown under a microscope before and after receiving one of the so-called vaccines. The microscope of the blood sample taken afterwards shows evidence of rouleau, and also include what appear to be nano structures and possibly graphene oxide which appears to move and self-assemble, e.g., see the websites: https://fb.watch/aahyn6bhto/; https://www.facebook.com/nastasiagraceofficial/videos/233 133388865722; https://www.facebook.com/103214055247397/posts/206787971556671/; and, https://youtu.be/WFh7-6t34M. Karen Kingston, a former Pfizer employee also discusses the origins of the so-called “vaccines” and graphene oxide content: https://rumble.com/vkgdq7-deadly-shots-former-pfizer-employee-confirms-poison-in-covid-vaccine.html. The late Dr. Andreas Noack was an expert on the subject of graphene oxide substances who sounded an alarm regarding their inclusion in the so-called “vaccines.” In this regard, he discussed that graphene oxide and/or graphene hydroxide are stronger than steel and harder than diamond. Further, he suggested that because these substances can be a single molecule in thickness the combination of their ultra-thin profile, strength, and hardness can make pieces of these substances behave like small razor blades if and when they are introduced into an individual's bloodstream, and so could then cause internal bleeding. As a result, he considered that well-conditioned athletes such as soccer players who do a lot of running and have elevated pulse rates could be at higher risk of suffering injury due to the so-called “vaccines.” The following website includes the article and video entitled “German Whistleblower, Dr. Andreas Noack Was Murdered,” Dec. 21, 2021, and his video about the dangers of graphene oxide/graphene hydroxide, but also a video by his wife or partner after his death: https://seemorerocks.is/german-whistleblower-dr-andreas-noack-was-murdered/.

Other doctors and researchers have confirmed the presence of graphene oxide in the COVID-19 vaccines, e.g., see “Detection Of Graphene In COVID19 Vaccines:” Research Gate, November, 2021, Dr. Pablo Campra, University de Almeria, Micro-Raman Spectroscopy, which provides a pdf copy of his lab report: https://www.google.com/url?sa=i&source=web&cd=&ved=2ahUKEwiBsgfXt8j1AhXCk YkEHYwBBbQQ3YkBegQIARAD&url=https%3A%2F%2Fwww.xesearchgate.net %2F publication%2F355979001_DETECTION_OF_GRAPHENE_IN_COVID19_VACCINE S&psig=AOvVaw0Lkkrj99B6 XNgDzodJrR-&ust=1643045979701437. Further, also see the pdf copy of Dr. Robert Young's lab report which can be found with the article “American Scientists Confirm Toxic Graphene Oxide, and More, in Covid Injections:” https://rightsfr:eeIoms.wordpress.con2021/09/02/american-scientists-confirm-toxic-graphene-oxide-and-more-in-covid-injections/. The following article discusses that Japan found metallic particles in the Moderna so-called “vaccines,” and returned 2,600,000 doses to the company: https://www.orwell.city/2021/09/japan.html, and also see https://youtu.be/fXwhQtiV5uw.

Given the findings of Dr. Zandre' Botha under the microscope, and the lab results of Dr. Pablo Campra who performed Micro-Raman Spectroscopy analysis of the Moderna, Pfizer, and Johnson & Johnson so-called “vaccines,” and also the lab results of Dr. Robert Young, it appears that some form of graphene oxide or graphene hydroxide is present in some of the COVID-19 “vaccines.” Some individuals speculate that it was included to serve as a carrier or adjuvant. At this time, there are many unanswered questions with regards to what the aforementioned graphene substances can do when injected into an individual and patient. How much of the graphene material stays lodged in muscle tissue, and how much enters the bloodstream could depend on whether the injection hit or was proximate to a vein or artery. Does the graphene material migrate and then become lodged in other organs and tissues like the liver and kidneys? Does the graphene material migrate to areas associated with constant and relatively high electrical activity in the body like the heart and brain? Would it be counterproductive to ingest substances which could possibly break down graphene oxide material into small bits which could potentially migrate and lodge in other areas of the body? Does it eventually break down and become eliminated? At this time, the answers to these questions are not known. However, Senator Ron Johnson of Wisconsin held a Congressional Hearing on Jan. 24, 2020 which included a panel of doctors, researchers, and practitioners who discussed COVID-19, and it was then made clear by several members of the panel that the so-called “vaccines” do migrate from the injection site and into multiple human organs where they can cause health problems, e.g., see the testimony of Dr. Peter McCullough at 4:50 minutes in the Congressional Hearing on the website: https://youtu.be/asw_FBipVpg. The pharmaceutical companies have so far denied that graphene substances have been included in their so-called “vaccines.” In this regard, one can either choose to believe what some of the pharmaceutical companies have represented, or what microscopic examination and several other independent laboratory test results have shown.

The patents of Moderna, Inc. relating to their so-called “vaccine” are provided on their website: https://www.modernatx.com/patents. In order to find the patents and patent applications of Johnson & Johnson relating to their so-called “vaccines” individuals may search in the future for those patents of its subsidiary and assignee Janssen Pharmaceuticals using the U.S. Patent and Trademark Office's website: uspto.gov. A list of patents of Pfizer, Inc. relating to their so-called COVID-19 “vaccine” can be found on the website: “Pfizer and The Medicines Patent Pool (MPP) Sign Licensing Agreement for COVID-19 Oral Antiviral Treatment Candidate to Expand Access in Low- and Middle-Income Countries,” that is, if an individual clicks on “access the license agreement,” then clicks on the license agreement and scrolls to near the end of the document to the Appendix 2: https://www.pfizer.com/news/press-release/press-release-detail/pfizer-and-medicines-patent-pool-mpp-sign-licensing. In this regard, see U.S. patent application Ser. No. 17/395,139 which matured as U.S. Pat. No. 11,351,149 B2. If graphene oxide substances, and specific adjuvants are not recited in the relevant patents of the pharmaceutical companies, but they are in fact present in their so-called “vaccines” the patents could possibly be held to be invalid because of the pharmaceutical companies' failure to provide a full disclosure. Furthermore, the companies could possibly be held liable for “willful misconduct” by not listing or disclosing the substances to health officials and the general public and/or later denying that those substances have been and continue to be present in their so-called “vaccines.” As a group, the pharmaceutical companies that made the COVID-19 so-called “vaccines” have not been transparent regarding the contents of their products, nor the findings of their research studies. As a result, even today members of the general public do not know what was contained in the so-called “vaccines.”

While the contents of the COVID-19 so-called “vaccines” may not be known to members of general public, it should be noted that aluminum has been and continues to be used as an adjuvant in many other vaccines. Aluminum is believed or known by numerous individuals to be associated with adverse health effects including but not limited to Alzheimer's disease, and Autism, e.g., see the book entitled: “How To End the Autism Epidemic,” by J. B. Hanley. Mercury had been used as an adjuvant in vaccines for many years and was allegedly discontinued because it was recognized as being hazardous to human health, but it is nevertheless still being included in some vaccines as an ingredient in Thimerosal which is used a preservative against spoilage.

During the past two years, there have been an unusually high number of athletes who have suffered health emergencies, as discussed in the following articles: “At Least 69 Athletes Collapse In One Month, Many Dead:” https://dpbh.nv.gov/uploadedFilcs/dpbhnvgov/content/Boards/BOH/Meetings/2021/Public%20Comments%20324%20to%20328.pdf; “Report: 75 European Fully Vaccinated Athletes Have Died Or Became Seriously Ill From “Sudden” Heart Attacks In The Past 5 Months:“https://newzworldtoday.com/report-75-european-fully-vaccinated-athletes-have-died-or-became-seriously-ill-from-sudden-heart-attacks-in-the-past-5-months/; and, “Pfizer and Moderna Vaccines are Probably Behind Heart Failures of Professional Athletes in Europe:” https://tfglobalnews.com/2021/11/10/pfizer-and-moderna-vaccines-are-probably-bchind-hcart-failures-of-profcssional-athletes-in-europe/arn/.Some medical doctors and researchers think the COVID-19 so-called “vaccines” are possibly responsible for these and other health emergencies. Besides numerous high-profile cases involving the sudden death of athletes, many other individuals who have received one of the Covid-19 so-called “vaccines” have later died from blood clots. However, the blood clots are abnormal and not composed of red blood cells, but instead largely of white blood cells, platelets, and other matter. In this regard, see the following video entitled “Died Suddenly.” https://rumble.com/v1wac7i-world-premier-died-suddenly.html. At this time, it appears the cause of these unusual blood clots could possibly be due to one or more of the following: higher than normal concentrations of one or more metals, graphene oxide, nanostructures, toxins such as toxic peptides, the COVID-19 virus, and pre-existing infections or other health conditions.

In this regard, Dr. Bryan Artis and Mike Adams who has an ISO lab have stated that several labs in China, France, and Italy have detected snake venom and other toxins in victims of COVID-19: e.g., see https://www.stewpters.com>video>2022>11>dr-jane-ruby-show-ardis-and-adams-blow-open-truth-on-mass-genocide-2, and, Dr. Jane Ruby Show: Ardis and Adams Blow Open Truth On Mass Genocide survivethenews.com EXCLUSIVE: Self-assembling vaccine clot biostructures harvest conductive metals from your blood—preliminary ICP-MS analysis results released—Survive the News. Mike Adams and Harrison Smith have also discussed finding unusual things in people's bloodstream: https://forbiddenknowledgctv.net/microscopic-video-of-engincered-bio-structures-removed-from-blood-vessels/; https://www.stewpeters.com, video>2022>08>exclusive-horrific-images-circuits-in-covid-iab-internet-routcr-causcs-circuits-to-self-assemble; Exclusive Horrific Images: Circuits In Covid Jab Internet . . . -Stew Peters.

According to Dr. Bryan Artis and Mike Adams synthetic snake venom peptides were possibly used to cleave normal RNA and insert the mRNA segment in making and using the COVID-19 so-called “vaccines.” In the video provided below, Mike Adams shows how he typically prepares organic matter for mass spectroscopy analysis, and in this case the sample is a white blood clot that has been taken from a deceased individual who had taken one of the Covid-19 mRNA so-called “vaccines.” https://www.bitchute.com/video/YAoeEIPKR6Dt/. It can be clearly seen that when a portion of the white blood clot is added to nitric acid some of the sample oxidizes and turns black in an exothermic reaction. Adams states that organic matter normally does not do this. What does do this is a form of inorganic matter, e.g., aluminum does this when it is placed in nitric acid as shown in the following video and demonstration: https://youtu.be/T1SdumDd-0s. Here is the chemical formula that is associated with this exothermic reaction: Aluminium+nitric acid=aluminium nitrate+hydrogen, that is, Al+HNO₃=Al NO₃+H, e.g., see https://pubchem.ncbi.nlm.nih.gov/compound/Aluminium-nitrate.

Edward Dowd is a former analyst for the Blackrock Investment firm who is studying the emerging data on excess mortality. He has written a book entitled: “Cause Unknown:” The Epidemic of Sudden Deaths in 2020 2021,” and observes that the sudden death epidemic has not stopped. See the websites: https://www.redvoicemedia.com/2022/11/cause-unknown-excess-mortality-the-epidemic-of-sudden-death-ed-dowd/ref/8/, https://fb.watch/gxL.oBY9bT/, and the data on excess mortality which is provided on his website: theyliedpeopledied.com/. According to the insurance data and actuary tables which Mr. Dowd has studied all-cause mortality in 2021 and 2022 was between 20-40 percent higher than average, and the upward trend of regarding disabilities and all-cause mortality continues to increase. One of the dangers possibly associated with the COVID-19 so-called “vaccines” is a phenomenon known as Antibody Dependent Enhancement (ADE), e.g., see https://en.wikipedia.org/wiki/Antibody-dependent enhancement. Again, Senator Ron Johnson of Wisconsin held a Congressional Hearing on Jan. 24, 2020 which included a panel of doctors, researchers, and practitioners and several individuals then expressed their concerns that the COVID-19 so-called “vaccines” can possibly comprise the human immune system and its ability to eliminate of cancer cells, e.g., see the testimony beginning at 4:58 minutes in the Congressional Hearing on the website: https://youtu.be/asw_FBipVpg. Further, data from the CDC website reveals that there have been approximately 1.5 million COVID-19 so-called “vaccine” injuries, over 190,000 hospitalizations, and over 34,000 deaths, and the actual numbers are believed to be much higher.

If some of the articles, videos, or websites recited above are found by the reader to have been removed, they should still be accessible using the Internet Archive WayBackMachine website: https://web.archive.org/.

Millions of individuals around the world have now taken the COVID-19 so-called “vaccines” and boosters. Because there is reason to believe that some or all of them possibly include substances which can pose health risks, there is need for a structure, device, method, and technique for removing foreign substances from the blood of individuals who have received one or more of the COVID-19 shots, or who otherwise have undesired metals, graphene oxide, nanostructures, pathogens, or toxins in their bloodstreams, and who wish to reduce their risk of suffering health problems now and in the future.

Accordingly, the present disclosure is directed to structures, devices, methods, and techniques for removing undesired foreign matter, such as metals, graphene oxide, nanostructures, pathogens, and toxins from liquids and human blood.

### SUMMARY

A first aspect of the present disclosure is a filter device for separating and removing undesired mater from blood or a component of blood including a chamber having a top side, a bottom side, a lateral side and a central axis; the chamber including a cavity; an outside height dimension between the top side and the bottom side of the chamber, and maximum width dimension; an inlet disposed on the top side and an outlet disposed on the bottom side; a filter disposed in the cavity; the filter including at least one of a mesh, a screen, and a filter media capable of capturing, trapping, or adhering the undesired matter being disposed on a plane which is orientated perpendicular to the central axis, whereby the blood or component of blood can be filtered in the chamber between the inlet and the outlet.

Optionally, the outside height dimension is equal to or greater than the maximum width dimension.

Optionally, the lateral side is curved.

Optionally, the filter device has a cylindrical shape.

Optionally, the inlet and the outlet of are removable.

Optionally, the inlet is configured to be aligned with the outlet and also about the central axis.

Optionally, the at least one of the mesh, the screen, and the filter media forms a layer, and the filter includes a plurality of layers.

Optionally, the at least one of the mesh, the screen, and the filter media further includes a border.

Optionally, the at least one of the mesh, the screen, and the filter media includes a plurality of wires.

Optionally, the at least one of the mesh, the screen, and the filter media includes a convoluted configuration.

Optionally, the plurality of layers are configured in a nested configuration.

Optionally, the filter device further includes a magnet device disposed proximate to the filter for providing an electromagnetic field.

Optionally, the magnet device includes at least one permanent magnet or electromagnet disposed proximate to at least one of the top side and the bottom side of the chamber, whereby the undesired matter can be captured, trapped, or adsorbed in the filter.

Optionally, the magnet device includes at least one permanent magnet or electromagnet disposed proximate to the lateral side of the chamber, whereby the undesired matter can be captured, trapped or adsorbed in the filter.

Optionally, the magnetic device includes a first magnet having a first positive pole and a first negative pole, and a second magnet having a second positive pole and a second negative pole, the first magnet being configured relative to the second magnet such that the first positive pole of the first magnet is disposed opposite the second negative pole of the second magnet.

Optionally, the magnet device has an electromagnetic field which is in the range between 0.01-1.0 Tesla.

Optionally, the magnet device includes an electromagnet including a coiled wire.

Optionally, the magnet device includes an electromagnet including a wire coiled around a conductive core.

Optionally, the chamber further includes a support structure for removably securing the magnet device.

Optionally, the filter is configured to have an electrical charge.

Optionally, the filter includes a plurality of magnetic beads.

Optionally, the filter includes a screen or pore size smaller than about 1.5 microns, or 15 microns, or 30 microns.

Optionally, the undesired matter includes at least one of a metal, graphene oxide, graphene hydroxide, magnetic graphene oxide, an electronic chip or other electronic device, a pathogen, and a toxin.

A second aspect of the present disclosure includes a filter device for separating and removing undesired matter from blood or a component of blood including: a chamber having a top side, a bottom side, a lateral side, and a central axis; the chamber including a cavity; an outside height dimension between the top side the bottom side of the chamber, a maximum width dimension; an inlet disposed on the top side and an outlet disposed on the bottom side; a filter disposed in the cavity; the filter including at least one of a mesh, a screen, and a filter media capable of capturing, trapping, or adsorbing the undesired matter; the at least one of the mesh, the screen, and the filter media being disposed concentrically about the central axis, whereby the blood or component of blood can be filtered in the chamber between the inlet and the outlet.

Optionally, the lateral side is curved.

Optionally, the filter device has a cylindrical shape.

Optionally, the inlet and the outlet of are removable.

Optionally, the inlet is configured to be aligned with the outlet and also about the central axis.

A third aspect of the present disclosure includes a filter device for separating and removing undesired matter from blood or a component of blood including: a chamber having a top side, a bottom side, a lateral side, and a central axis; the chamber including a cavity; an outside height dimension between the top side and the bottom side of the chamber, a maximum width dimension; an inlet disposed on the top side and an outlet disposed on the bottom side; the inlet being in fluid communication with at least one channel which is in fluid communication with the cavity; the chamber including a perforated tube orientated parallel to and about the central axis; the perforated tube being in fluid communication with the outlet; a filter disposed in the cavity of the chamber; the filter including at least one of a mesh, a screen, and a filter media capable of capturing, trapping, or adsorbing the undesired matter; the at least one of the mesh, the screen, and the filter media being disposed concentrically about and parallel to the central axis; whereby the blood or component of blood can be filtered in the chamber between the inlet and the outlet.

Optionally, the lateral side is curved.

Optionally, the filter device has a cylindrical shape.

Optionally, the inlet and the outlet of are removable.

Optionally, the inlet is configured to be aligned with the outlet and also about the central axis.

A fourth aspect of the present disclosure includes a filter device for separating and removing undesired matter from blood or a component of blood including: a chamber having a top side, a bottom side, a curved lateral side, and a cylindrical shape; the chamber including a cavity; an outside height dimension between the top side and the bottom side of the chamber, a maximum width dimension; an inlet disposed on the top side and an outlet disposed on the bottom side; the inlet configured to be aligned with the outlet and about the central axis; the chamber including a first wall having a first outside surface and a first inside surface and having a first radius; the chamber including a second wall having a second outside surface and a second inside surface and having a second radius; the second wall being concentric to the first wall and the second radius being less than the first radius; the second wall defining a first space in the cavity of the chamber between the first inside surface of the first wall and the second outside surface of the second wall; the second wall defining a second space in the cavity of the chamber between opposite portions of the second inside wall; a filter including at least one of a mesh, a screen, and a filter media capable of capturing, trapping, or adsorbing the undesired matter disposed in the cavity of the chamber in the second space; and, a magnetic device disposed in the cavity of the chamber in the first space, whereby the magnetic device can impart an electromagnetic field upon the filter and the undesired matter, whereby the blood or component of blood can be filtered in the chamber between the inlet and the outlet.

A fifth aspect of the present disclosure includes a filter device for separating and removing undesired matter from blood or a component of blood including: a chamber having a top side, a bottom side, and a lateral side; the chamber including a cavity; an outside height dimension between the top side and the bottom side of the chamber, a maximum width dimension; the chamber including an inlet and an outlet; the chamber including a spiral channel which descends from proximate to the top side to proximate to the bottom side; a filter including at least one of a mesh, a screen, and a filter media capable of capturing, trapping, or adsorbing the undesired matter disposed within the spiral channel, whereby the length of the flow path of the blood or blood component is greater than the outside height dimension, and the blood or component of blood can be filtered in the chamber between the inlet and the outlet.

Optionally, the spiral channel descends from proximate to the top side to proximate to the bottom side in a plurality of portions which alternate in slope in the range between 0/1 and 10/1.

Optionally, the lateral side is curved.

Optionally, the filter device has a cylindrical shape.

Optionally, the inlet and the outlet of are removable.

Optionally, the filter device has a central axis.

Optionally, the inlet is configured to be aligned with the outlet and also about the central axis.

A sixth aspect of the present disclosure includes a filter device for separating and removing undesired matter from blood or a component of blood including: a chamber having a top side, a bottom side, and a lateral side; the chamber including a cavity; an outside height dimension between the top side and the bottom side of the chamber, a maximum width dimension; an inlet disposed on the top side and an outlet disposed on the bottom side; the chamber including a plurality of baffles defining a plurality of channels and openings; each of the plurality of channels being in fluid communication with each other of the plurality of channels through the openings; one or more filters including at least one of a mesh, a screen, and a filter medium disposed within the plurality of channels; whereby the length of the flow path of the blood or blood component is greater than the outside height dimension and the blood or component of blood can be filtered in the chamber between the inlet and the outlet.

Optionally, the lateral side is curved.

Optionally, the filter device has a cylindrical shape.

Optionally, the inlet and the outlet of are removable.

Optionally, the filter device has a central axis.

Optionally, the inlet is configured to be aligned with the outlet and also about the central axis.

A seventh aspect of the present disclosure includes a method of treating an individual having undesired matter dispersed in their blood, the method including the steps of: inserting a first needle into a first vein or artery of the individual at a first puncture site and connecting the first needle to tubing, a stop valve, a filter device, an air bubble eliminator, and to a second needle which is inserted into a second vein or artery of the individual at a second puncture site; and, manipulating the stop valve to initiate the flow of blood from the first puncture site through the filter device and reinfusing filtered blood through the second puncture site.

Optionally, the method further includes a magnet device including at least one permanent magnet or electromagnet for providing an electromagnetic field disposed proximate to the filter.

Optionally, the magnet device includes a first magnet having a first positive pole and also a first negative pole, and a second magnet having a second positive pole and also a second negative pole, the first magnet being configured relative to the second magnet such that the first positive pole of the first magnet is disposed opposite the second negative pole of the second magnet.

Optionally, the electromagnetic field of the magnet device is in the range between 0.01-1.0 Tesla.

Optionally, the magnet device further includes at least one selection control for controlling at least one of the following electrical or electromagnetic variables: the voltage and current, the frequency and wavelength, the waveform, the polarity, the source of electrical power being either AC versus DC, and an on and off switch.

Optionally, the filter device is configured to have an electrical charge.

Optionally, the filter device further includes a plurality of magnetic beads, whereby the undesired matter is separated from the blood and captured, trapped, or adsorbed in the filter device.

Optionally, the undesired matter includes at least one of a metal, graphene oxide, graphene hydroxide, magnetic graphene oxide, an electronic chip or other electronic device, a pathogen, and a toxin.

Optionally, the flow of blood is performed at one of the following rates: 0.1-10 L/hr, 1-10 L/hr, 3-10 L/hr, or 5-10 L/hr.

Optionally, the flow of blood and reinfusion is performed in a continuous mode, or a cyclic mode.

Optionally, the filter includes a screen or pore size smaller than 1.5 microns, or 15 microns, or 30 microns.

Optionally, the inlet of the filter device and the outlet of the filter device are removable.

An eighth aspect of the present disclosure is a method of treating an individual having undesired matter dispersed in their blood, the method including the steps of: inserting a first needle into a first vein or artery of the individual at a first puncture site and connecting the first needle to tubing, a stop valve, a filter device, a pump, an air bubble eliminator, and to a second needle which is inserted into a second vein or artery of the individual at a second puncture site; and, manipulating the stop valve to initiate the flow of blood from the first puncture site through the filter device and reinfusing filtered blood through the second puncture site.

Optionally, the method further includes connecting and using at least one analyte sensor between the first needle and the filter device.

Optionally, the method further includes connecting and using a blood or blood portion dispersion device including a window between the first needle and the filter device.

Optionally, the method further includes deploying and using at least one optical device between the first needle and the filter device.

Optionally, the method further includes deploying and using a first blood or blood component conditioning device between the first needle and the filter device.

Optionally, the method further includes deploying and using a second blood or blood component conditioning device proximate to the filter device.

Optionally, the second blood or blood component conditioning device includes a magnet device including at least one permanent magnet or electromagnet for providing an electromagnetic field.

Optionally, the magnet device includes a first magnet having a first positive pole and a first negative pole, and a second magnet having a second positive pole and a second negative pole, the first magnet being configured relative to the second magnet such that the first positive pole of the first magnet is disposed opposite the second negative pole of the second magnet.

Optionally, the electromagnetic field of the magnetic device is in the range between 0.01-1.0 Tesla.

Optionally, the method further includes deploying and using a third blood or blood component conditioning device between the filter device and the second needle.

Optionally, the method further includes deploying and using a computer or other electronic medical device including a monitor and having a wired or wireless connection to at least one of a stop valve, an air bubble eliminator, a filter, a pump, an analyte monitoring device, an optical device, and a blood or blood component conditioning device.

Optionally, the method further includes an optical device which is at least one of a microscope, a stereo microscope, and an electron microscope.

Optionally, the blood or blood component conditioning device includes at least one selection control for controlling at least one of the following electrical or electromagnetic variables: the voltage and current, the frequency and wavelength, the waveform, the polarity of at least one electromagnet, the source of electrical power being either AC versus DC, and an on and off switch.

Optionally, the filter device is configured to have an electrical charge whereby the undesired matter can be separated and removed from the blood and be captured, trapped, or adsorbed in the filter device.

Optionally, the filter device further includes a plurality of magnetic beads whereby the undesired matter can be separated and removed from the blood and captured, trapped, or adsorbed in the filter device.

Optionally, the undesired matter includes at least one of a metal, graphene oxide, graphene hydroxide, magnetic graphene oxide, an electronic chip or other electronic device, a pathogen, and a toxin.

Optionally, the flow of blood is performed at one of the following rates: 0.1-10 L/hr, 1-10 L/hr, 3-10 L/hr, or 5-10 L/hr.

Optionally, the flow of blood and reinfusion is performed in a continuous mode, or a cyclic mode.

Optionally, the filter includes a screen or pore size smaller than 1.5 microns, or 15 microns, or 30 microns.

Optionally, the inlet of the filter device and the outlet of the filter device are removable.

Optionally, the pump is a portion of medical device selected from the group of medical devices consisting of an apheresis device, a plasmapheresis device, an extracorporeal blood purification device, a hemofiltration device, a continuous veno-venous hemofiltration device, a hemodiafiltration device, an ultrafiltration device, and a hemodialysis or dialysis device.

Optionally, the filter is a portion of a medical device selected from the group of medical devices consisting of an apheresis device, a plasmapheresis device, an extracorporeal blood purification device, a hemofiltration device, a continuous veno-venous hemofiltration device, a hemodiafiltration device, an ultrafiltration device, and a hemodialysis or dialysis device.

### BRIEF DESCRIPTION OF THE DRAWINGS

FIG. 1 is a side cross sectional view of a filter device.

FIG. 2 is a side cross sectional view of an alternative embodiment of a filter device including a magnetic device.

FIG. 3 is a top view of a screen portion of a filter for use in a filter device.

FIG. 4 is a top view of an alternative embodiment of a filter for use in a filter device.

FIG. 5 is a top perspective view of the alternate embodiment of a filter for use in a filter device.

FIG. 6 is a side cross sectional view of an alternative filter device including an alternative magnetic device.

FIG. 7 is a top perspective view of an alternative embodiment of a filter for use in a filter device.

FIG. 8 is a top view of a portion of an alternative embodiment of a filter for use in a filter device.

FIG. 9 is a side cross-sectional view of an alternative embodiment of a filter device.

FIG. 10 is a top view of the filter shown in FIG. 7.

FIG. 11 is a cross-section view taken along line 11-11 of the blood dispersion device shown in FIG. 16.

FIG. 12 is a cross-sectional view taken along line 12-12 of an alternative embodiment of the filter device shown in FIG. 15.

FIG. 13 is a perspective view of an alternative electromagnet disposed around a tube.

FIG. 14 is a side view of an alternative electromagnet disposed around a filter device.

FIG. 15 is a side cross-sectional view of an alternative embodiment of a filter device including an alternative electromagnet device.

FIG. 16 is a top view of a blood dispersion device.

FIG. 17 is a side view of a T-shaped alternative embodiment of a filter device.

FIG. 18 is a side view of a L-shaped alternative embodiment of a filter device.

FIG. 19 is a side view of three components of a kit for making a blood infusion or transfusion.

FIG. 20 is a side cross-sectional view of an alternative embodiment of a filter device which includes a plurality of internal baffles and openings.

FIG. 21 is a side cross-sectional view of an alternative embodiment of a filter device which includes a spiral structure.

FIG. 22 is a side cross sectional view of a filter device generally similar to the filter device shown in FIG. 1, but which further includes a removable top side and seal.

FIG. 23 is a top view of an alternative T-shaped embodiment of a filter device which includes four quadrant portions.

FIG. 24 is a side cross sectional view taken along line 24-24 of the alternative T-shaped embodiment of a filter device shown in FIG. 23.

FIG. 25 shows an extracorporeal blood filtering device and method which can include in complete combination or various alternative partial combinations, a first needle disposed at a first puncture site, tubing, a valve or roller clamp, a filter device, an air bubble eliminator, and a second needle disposed at a second puncture site on the body of an individual and patient.

FIG. 26 shows an alternative embodiment of an extracorporeal blood filtering device and method which can include in complete combination or various alternative partial combinations, a first needle disposed at a first puncture site, tubing, a valve or roller clamp, a filter device, an air bubble eliminator, a second needle disposed at a second puncture site on the body of an individual and patient, and also a conditioning device, a plurality of wires, and a computer or other medical device which includes a monitor screen and a plurality of selection controls including a keyboard.

FIG. 27 shows an alternative embodiment of an extracorporeal blood filtering device and method which can include in complete combination or various alternative partial combinations, a first needle disposed at a first puncture site, tubing, a stop valve, a filter device, a pump device, an air bubble eliminator, a second needle disposed at a second puncture site on the body of an individual and patient, and also a conditioning device, a plurality of wires, and a computer or other medical device including a monitor screen and a plurality of selection controls including a keyboard.

FIG. 28 shows an alternative embodiment of an extracorporeal blood filtering device and method which can include in complete combination or various alternative partial combinations, a first needle disposed at a first puncture site, tubing, a valve or roller clamp, a filter device, a pump device, an air bubble eliminator, at least one analyte sensor device, a second needle disposed at a second puncture site on the body of an individual and patient, and also a conditioning device, a plurality of wires, and a computer or other medical device including a monitor screen and a plurality of selection controls including a keyboard.

FIG. 29 shows an alternative embodiment of an extracorporeal blood filtering device and method which can include in complete combination or various alternative partial combinations, a first needle disposed at a first puncture site, tubing, a valve or roller clamp, a filter device, a pump device, an air bubble eliminator, a second needle disposed at a second puncture site on the body of an individual and patient, a first conditioning device, a second conditioning device, a third conditioning device, a first analyte sensor device, a second analyte sensor device, a possible third analyte sensor device, a plurality of wires, and a computer or other medical device including a monitor screen and plurality of selection controls including a keyboard.

FIG. 30 shows an alternative embodiment of an extracorporeal blood filtering device and method which can include in complete combination or various alternative partial combinations, a first needle disposed at a first puncture site, tubing, a valve or roller clamp, a filter device, a pump device, an air bubble eliminator, a second needle disposed at a second puncture site on the body of an individual and patient, a first conditioning device, a second conditioning device, a third conditioning device, a first analyte sensor device, a second analyte sensor device, a third analyte sensor device, a first blood dispersion device, a second blood dispersion device, a first optical monitor, a second optical monitor, a possible third optical monitor, a plurality of wires, and a computer or other medical device including a monitor screen and a plurality of selection controls including a keyboard.

### DETAILED DESCRIPTION

Human blood includes four main components: plasma, red blood cells, white blood cells, and platelets. In this regard, the composition of human blood is about 45% blood cells and platelets, and about 55% plasma. An average man has about 12 pints of blood, and average woman about 9 pints of blood. With regards to the size and scale used to discuss blood and its various components one micron (μm) equals 1000 nanometers (nm) and the average size of red blood cells is normally in the range between 6-9 microns or 6,000-9,000 nm. The average size of small white blood cells is in the range between 7-8 microns or 7,000-8,000 nm, whereas the average size of most white blood cells is in the range between 10-15 microns or 10,000-15,000 nm, but a few white blood cells are as large as 15-30 microns or 20,000-30,000 nm. The average size of platelets is typically between 1.5-4 microns or 1,500-4,000 nm. For reference purposes, the size of a hydrogen atom is 0.1 nm; the size of a silicon atom is about 0.2 nm; the size of a DNA molecule is about 1 nm; the size of the COVID-19 virus is between 60-140 nm and it is negatively charged, and, the width of a human hair is about 80-100 microns or 80,000-100,000 nm.

Materials in the graphene family such as graphene oxide, graphene hydroxide, magnetic graphene oxide can be made one molecule layer thick which is approximately between 1-10 nm, but their length or sheet and particle size of can be anywhere between several nanometers to microns. These particles are typically electrically conductive and have a negative charge. At this time, the brand and specific type and size of the graphene particles which have apparently been included in the COVID-19 so-called “vaccines” are not known because the pharmaceutical companies did not list the substances as ingredients, and have denied that such particles have been included. However, more than one researcher has found evidence of foreign particles which belong to the graphene family in the so-called “vaccines.” It is not known whether all of the pharmaceutical companies used foreign particles of the same graphene family or different types and sizes of these particles in their so-called “vaccines.” Metals and other electrically conductive or magnetic particles, and including those in the graphene family such as graphene oxide or graphene hydroxide can sometimes be attracted to one another and then congregate, aggregate, and/or self-assemble. Such particles are responsive to electromagnetic radiation, magnetic fields, and including cell phones using 4G and 5G, and they can possibly constitute antennas on the micron and nanometer scales. Accordingly, the particle size of graphene oxide or other like substances in human blood could potentially be anywhere in the range between 1-10 nm to one or more microns. Because these substances can congregate, aggregate, and self-assemble there is also a potential for obstruction and clogging to take place within the circulatory system and elsewhere in the human body. The single molecule thick structure of graphene oxide combined with the strength and hardness of the material can possibly also provide a hazard with regards to the particles cutting and causing hemorrhaging and blood clots to form within the human body. The potential risk can possibly be increased for those athletes who typically exercise at elevated heart rates such as runners and soccer players, but also for individuals having pre-existing heart and circulatory system issues. Moreover, there are also potential risks which can be associated with the effects of electronic devices such as cell phones and heart pacemakers being placed in close proximity to an individual having metal or other electrically conductive foreign particles such as graphene oxide in their bloodstream. Some individuals believe that graphene oxide can possibly be broken down in the human body and eliminated by eating certain foods and/or taking certain substances. However, insofar as graphene oxide is stronger than steel and harder than diamond, and in some sense, resembles a sheet of glass on a micron or nanometer scale, the idea of breaking a sheet of glass into smaller pieces which could possibly then potentially travel more easily and become lodged in more vulnerable places may not be prudent in view of some of the aforementioned risk factors. In summary, there is a need for targeting, separating, and removing foreign and undesired matter which could include, but not be limited to members of the graphene family from the blood of potentially millions of people in the United States, and also all around the world.

### Extracorporeal Blood Filtering Device

This disclosure is written with the recognition that the same medical practices, equipment and supplies are not necessarily available to everyone all around the world. Further, depending upon which so-called “vaccines” have been administered, and how many booster shots have been taken, different individuals around the world could have different amounts or types of foreign matter, such as metals, or materials of the graphene family, or other kinds of undesired matter in their bloodstream and bodies. Other forms of undesired matter could possibly also include markers, trackers, antennas, electronic chips and/or other electronic devices. For this reason, this application will disclose several different methods, techniques, and processes for possibly removing undesired matter from a fluid, blood portion or blood of individuals and patients.

Before any of the procedures and techniques discussed below would be conducted, a typical medical practice would be to first remove, observe, analyze and test a small sample of an individual and patient's 28 blood 10 in order to ascertain the presence of undesired matter 9, and also screen for other pre-existing health conditions. At that time, a medical practitioner would be able determine the individual or patient's 28 ABO blood group system blood type, Rh factor and establish a baseline reference point with regards to their blood 10 profile and general health. In this regard, the removal, examination, and possible filtering treatment and return of human blood 10 to an individual and patient 28 is and should be performed by a trained medical doctor, nurse, paramedics, phlebotomists, dialysis technicians, or qualified nursing staff.

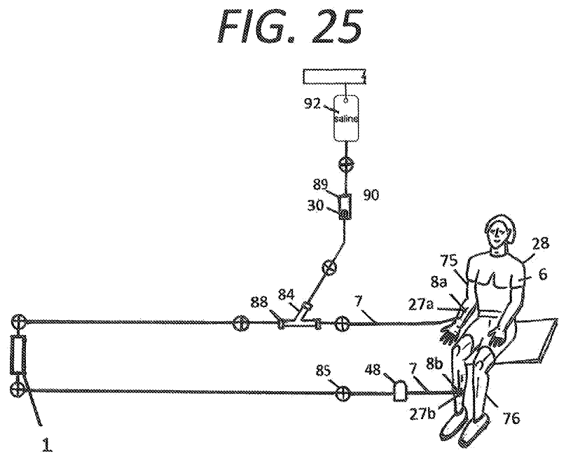

Several different alternative methods, techniques, and processes for possibly removing undesired matter from a fluid, blood portion or blood 10 of individuals and patients 28 will be disclosed and discussed with reference to drawing FIGS. 25-30 because the same medical expertise, methods, techniques, processes, equipment and supplies are not available to everyone all around the world. Some of these alternative methods, techniques, and processes would require fewer medical personnel and also less medical equipment, and so could be faster to perform and also less expensive than others.

Conventional Blood Infusion and/or Transfusion

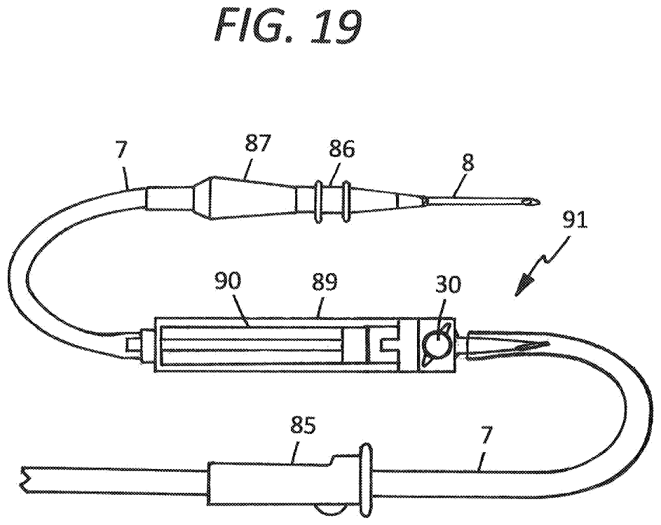

Common medical practice for the purpose of making a blood infusion and/or transfusion is to first locate one or more veins or arteries, and apply a tourniquet 3-4 inches above the desired puncture site 27a, sanitize the area where a needle 8a would be inserted using alcohol wipes, and then insert and secure the needle 8a. The size needle 8a which is typically used is 18 gauge, and made of a stainless-steel material. As shown in FIG. 19, the needle 8a can be connected to a straight union 86 made of a thermoplastic material such as an acrylic, a low-density polyethylene (LDPE), an acrylonitrile butadiene styrene (ABS), a polyvinyl chloride (PVC), a polyamide/Nylon (PA), a polypropylene (PP) which can be connected to a natural or latex rubber fitting 87 which in turn is connected to flexible tubing 7 which can be made of a clear thermoplastic material such as PVC. During a conventional blood infusion and/or transfusion procedure, a line including the aforementioned needle 8a, union 86, natural or latex rubber fitting 87, and tubing 7 is typically connected to the outlet 32c of a drip chamber 89. The drip chamber 89 is at least partially transparent and includes a flexible portion which can be used to manually pump a fluid therethrough in order to flush and at least partially fill the drip chamber 89. The drip chamber 89 includes an inlet 31c, an outlet 32c, and can and typically does include an integral valve 30 for venting air and also controlling the flow of a fluid, blood portion or blood 10. The drip chamber 89 also further includes a conventional filter 90 for removing possible blood clots and typically has a particle filtration size in the range between 170-260 microns. The inlet 31c of the drip chamber 89 can be and typically is connected by tubing 7 to the outlet 32y of a Y shaped union 84 which can be made and configured as a piece of Y tubing which includes three connectors 88 such as luer connectors 94 for joining and making fluid communication with three different lines of tubing 7. In this regard, one branch of the Y shaped union 84 can be and typically is connected by tubing 7 to the outlet 32b of a bag 92 of 0.9% saline solution which can be hung overhead and used to flush out the tubing 7 and any other devices which are going to be used in the procedure, and also possibly in order dilute the contents of a second bag 95 which can possibly include, e.g., whole blood, red blood cells, white blood cells, or plasma. Alternatively, when a bag 92 of 0.9% saline solution is not available, the individual and patient's 28 own blood 10 and/or the contents of the second bag 95 can be used to flush and/or fill the devices and lines that will be used during the blood infusion and/or transfusion procedure. The other branch of the Y shaped union 84 can be and typically is connected to a second bag 95 which can possibly include, e.g., whole blood, red blood cells, white blood cells, or plasma which is to be transfused. Three valves 30 in the form of roller clamps 85 which can be used to stop and/or control the rate of flow of these fluids are typically disposed on all of the tubes 7 leading to and from the Y shaped union 84, and a valve 30 or roller clamp 85 is also typically disposed on the tubing 7 which extends between the outlet 32c of the drip chamber 89 and the rubber or latex fitting 87, union 86 and needle 8a. When all of the above structures and connections have been made and the blood infusion and/or transfusion apparatus has been prepared, in a routine and non-emergency situation a medical professional will manipulate the valve(s) 30 and/or roller clamps 85 to initiate a blood infusion and/or transfusion of red blood cells, plasma, and/or platelets at a rate of 2 milliliters (mL)/kilogram (kg)/hour (hr) for the first 15 minutes, and then will typically increase the rate of flow to about 2-5 ml/kg/hr for red blood cells, and 4-10 ml/kg/hr for plasma, and or platelets.

### Extracorporeal Filtering of Blood

Common medical practice for the purpose of filtering and treating a blood portion and/or blood of an individual and patient is to first locate one or more veins or arteries, and apply a tourniquet 3-4 inches above the desired first puncture site 27a, sanitize the area where a first needle 8a would be inserted using alcohol wipes, and then insert and secure the first needle 8a. The size needle 8a which is typically used is 18 gauge and made of a stainless-steel material. The first needle 8a can be connected to a first straight union 86a made of a thermoplastic material such as an acrylic, a low density polyethylene (LDPE), an acrylonitrile butadiene styrene (ABS), a polyvinyl chloride (PVC), a polyamide/Nylon (PA), a polypropylene (PP) which can be connected to a first natural or latex rubber fitting 87a which in turn is connected to a piece of flexible tubing 7 which can be made of a clear thermoplastic material such as PVC. The tubing 7 can be and typically is connected to a Y shaped union 84 which can be configured as a piece of Y tubing which includes three connectors 88 such as luer connectors 94 for making fluid communication with three different lines and pieces of tubing 7. In this regard, one branch of the Y shaped union 84 can be connected using a piece of tubing 7 to the aforementioned tubing 7, rubber fitting 87 and union 86 structures which are connected to and in fluid communication with the first needle 8a. The other branch of the Y shaped union 84 can be possibly connected to a piece of tubing 7 which is connected to the outlet 32c of a drip chamber 89. The inlet 31c of the drip chamber 89 can be connected to the outlet 32b of a bag 92 of 0.9% saline solution which can be hung overhead and used to flush out the tubing 7 and any other devices which are going to be used in the procedure. The drip chamber 89 is at least partially transparent and includes a flexible portion which can be used to manually pump a fluid in order to flush and at least partially fill the drip chamber 89. The drip chamber 89 includes an inlet 31c, an outlet 32c, and can and typically does include an integral valve 30 for venting air and also controlling the flow of a liquid, blood portion or blood 10. The drip chamber 89 also further includes a conventional filter 90 for removing possible blood clots or other matter and typically has a particle filtration size in the range between 170-260 microns. Three valves 30 which can be in the form of roller clamps 85 can be used to stop and/or control the rate of flow of any given fluid are typically disposed on all of the tubes 7 leading to and from the Y shaped union 84, and including the tubing 7 which is disposed between the Y shaped union 84 and the rubber or latex fitting 87, union 86, and needle 8a. The outlet 32y of the Y shaped union 84 is typically connected to one end of piece of tubing 7 and the other end is connected to or otherwise placed in fluid communication with the inlet 31f of a filter device 4 which is configured to separate and remove undesired matter 9, such as metals, members of the graphene family, trackers, chips and other electronic devices. The outlet 32f of the filter device 4 can be and typically is connected to a piece of tubing 7 which is connected to or otherwise placed in fluid communication with the inlet 31a an air bubble eliminator 48. Another piece of tubing can be used to connect the outlet 32a of the air bubble eliminator 48 to the second natural or latex rubber fitting 87b which is connected to a second union 86b which is connected to a second needle 8b that is inserted at a second puncture site 27b. Alternatively, when a bag 92 of 0.9% saline solution is not available, the individual and patient's 28 own blood 10 can be used to flush and fill the devices and lines that will be used during the procedure. In that case, the Y shaped union 84 can be eliminated and the tubing 7 connected to the first natural or latex fitting 87a and first union 8a and first needle 8a can instead be connected to or otherwise placed in fluid communication with the inlet 31f of the filter device 1. When all of the above structures and connections have been made and the extracorporeal blood filtering and transfusion apparatus has been prepared, in a routine and non-emergency situation a medical professional will manipulate the valve(s) 30 and/or roller clamps 85 to initiate a blood transfusion of red blood cells, plasma, and/or platelets at a rate of 2 milliliters (mL)/kilogram (kg)/hour (hr) for the first 15 minutes, and then will typically increase the rate of flow to about 2-5 ml/kg/hr for red blood cells, and 4-10 ml/kg/hr for plasma, and or platelets.

Henceforth in this application, the structures which are sometimes referred to in the medical field and which have been independently recited in the specification as being a needle 8a, a union 86a, and a natural rubber or latex fitting 87a will be simply referred to as a needle or first needle in the claims. Likewise, the structures which have been independently recited as being a needle 8b, a union 86b, and a natural rubber or latex fitting 87b will be simply referred to as a second needle in the claims. Further, the structures which have been independently recited as being a part of drip chamber 89 which typically includes a transparent portion, a flexible portion which can be manipulated as a hand pump, a conventional blood filter, and also a valve 30 will be simply be referred to as a drip chamber in the claims. In addition, the structures which are sometimes referred to in the medical field as being an air bubble eliminator, or an air bubble filter, or an air bubble trap will be referred to as simply an air bubble eliminator 48 in the specification and air bubble eliminator in the claims. Examples of commercially available air bubble eliminators include the GVS SpeedFLow® 0.2 μm filter made by GVS Filtration, Inc. of Bologna, Italy which is available from TrueCare Biomedix USA Inc., of South Miami, Florida, and the B. Braun 0.2 μm SUPOR® filter made by B. Braun Medical, Inc. of Bethlehem, Pennsylvania and B. Braun Melsunger AG, located in Melsungen, Germany.

The extracorporeal blood filtering device 33a-f, and related methods, techniques and processes can include the devices, structures, methods and techniques which are disclosed in FIG. 19, FIGS. 25-30, and others discussed herein. As shown in FIG. 25, a first needle 8a can inserted into a vein or artery of an individual and patient's 28 arm 75 and be secured proximate to the first puncture site 27a. One end of a first piece of tubing 7a can be connected to the needle 8a and the other end can be connected to the inlet 31v of a valve 30 or roller clamp 85 which can stop or regulate the flow of an individual and patient's 28 blood 10. One end of a second piece of tubing 7b can be connected to the outlet 32v of the valve 30 and the other end can be connected to the inlet 31f of a filter device 1. One end of a third piece of tubing 7c can connected to the outlet 32f of the filter device 1 and the other end can be connected to the inlet 31a of an air bubble eliminator 48. One end of a fourth piece of tubing 7d can be connected to the outlet 32a of the air bubble eliminator 48 and the other end can be connected to a second needle 8b which is can be inserted and secured proximate a second puncture site 27b on the individual and patient 28. The second puncture site 27b can be located at a second vertical elevation on a different arm 75 or leg 76 of the individual and patient 28 that is below the first vertical elevation of the first puncture site 27a so that the individual and patient's 28 own heart, blood pressure and gravity can be used to move their blood 10 through the filter device 1 and back into their bloodstream.

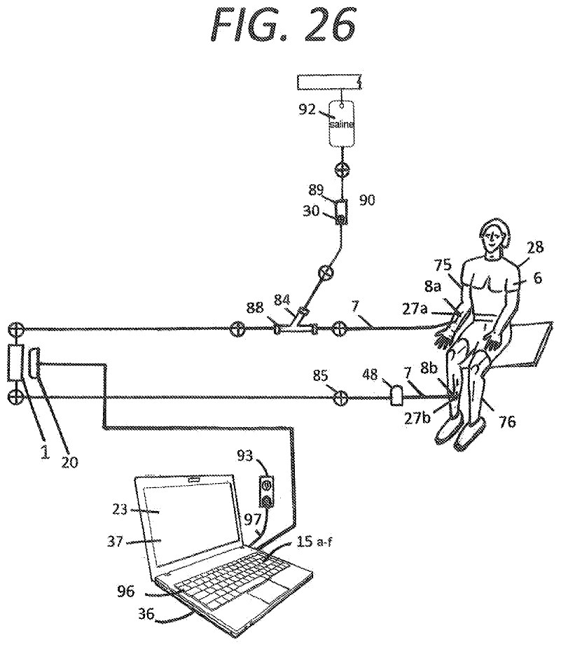

As shown in FIG. 26, the next simplest and alternative extracorporeal blood filtering device 33b, and related method, technique and process can be the same as that which has been disclosed above, but further includes a blood or blood portion conditioning device 20, hereinafter referred to as “conditioning device 20” or “second conditioning device 20,” which can include a magnet device 11 that is disposed either inside or proximate to the filter device 1 and/or filter 4 for attracting, directing, or otherwise manipulating the foreign undesired matter 9 so that it can be captured, mechanically trapped and/or adsorbed by the filter 4. The conditioning device can be connected by wire or wirelessly to a computer 36 or other medical device having a monitor screen 37 and a keyboard 96, touch pad, and mouse.

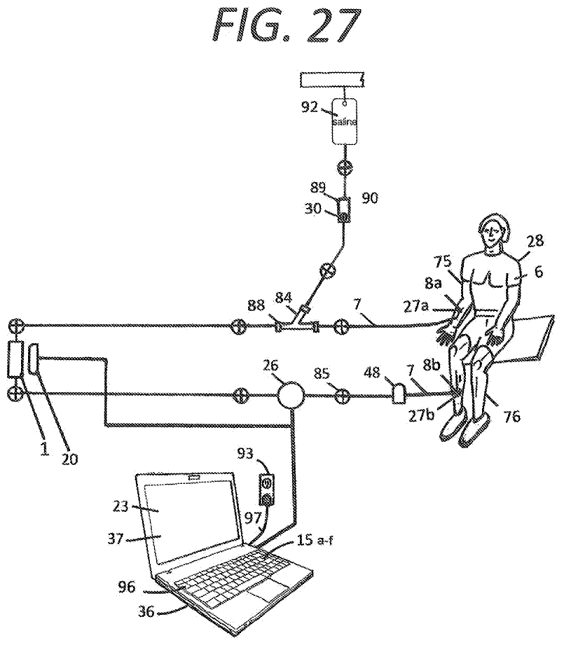

As shown in FIG. 27, the next simplest and alternative extracorporeal blood filtering device 33c, and related method, technique and process can be the same as that which has been disclosed above, but further includes a blood pump 26 disposed between the filter device 1 and the air bubble eliminator 48. Accordingly, as shown in FIG. 27, a first needle 8a can inserted into a vein or artery of an individual and patient's 28 arm 75 and be secured proximate to the first puncture site 27a. One end of a first piece of tubing 7a can be connected to the needle 8a and the other end can be connected to the inlet 31v of a valve 30 or roller clamp 85 which can stop or regulate the flow of an individual and patient's 28 blood 10. One end of a second piece of tubing 7b can be connected to the outlet 32v of the valve 30 and the other end can be connected to the inlet 31f of a filter device 1. One end of a third piece of tubing 7c can be connected to the outlet 32f of the filter device 1 and the other end can be connected to the inlet 21p of the blood pump 26. One end of a fourth piece of tubing 7d can be connected to the outlet 32b of the blood pump 26 and the other end can be connected to the inlet 31a of an air bubble eliminator 48. One end of a fifth piece of tubing 7e can be connected to the outlet 32a of the air bubble eliminator 48 and the other end can be connected to a second needle 8b which is can be inserted and secured proximate a second puncture site 27b on the individual and patient 28's arm 75 or leg 76. When a blood pump 26 is included, additional valves 30 or roller clamps 85 for stopping or regulating the individual and patient's blood 10 flow can be disposed before the inlet 31p and/or after the outlet 32p of the blood pump. The conditioning device 20 and blood pump 26 can be connected by wire or wirelessly to a computer 36 or other medical device having a monitor screen 37 and a keyboard 96, touch pad, and mouse.

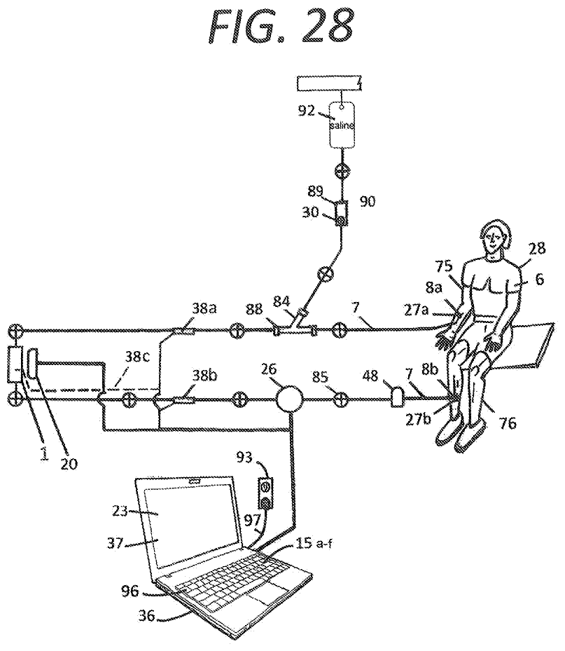

As shown in FIG. 28, the next simplest and alternative extracorporeal blood filtering device 33d, method, technique and process can be the same as that which has been disclosed above, but further includes an first analyte sensor device 38a for detecting the undesired matter 9 before the inlet 31f of the filter device 1, a second analyte sensor device 38c after the outlet 32f of the filter device 1 disposed before the needle 8b located at the second puncture site 27b so the amount and degree of success in removing the undesired matter 9 can be measured and known. Further, a third analyte sensor device 38c can possibly be disposed inside or proximate to the filter 1 for research purposes. Accordingly, as shown in FIG. 28, a first needle 8a can inserted into a vein or artery of an individual and patient's 28 arm 75 and be secured proximate to the first puncture site 27a. One end of a first piece of tubing 7a can be connected to the needle 8a and the other end can be connected to the inlet 31v of a valve 30 or roller clamp 85 which can stop or regulate the flow of an individual and patient's 28 blood 10. One end of a second piece of tubing 7b can be connected to the outlet 32v of the valve 30 and the other end can be connected to the inlet 31s of the first analyte sensor 38a. One end of a third piece of tubing 7c can be connected to the outlet 32s of the first analyte sensor 38a and the other end can be connected to the inlet 31f of a filter device 1. One end of a fourth piece of tubing 7d can be connected to the outlet 32f of the filter device 1 and the other end can be connected to the inlet 32p of the blood pump 26. One end of a fifth piece of tubing 7d can be connected to the outlet 32b of the blood pump 26 and the other end can be connected to the inlet 31s of the second analyte sensor 38b. One end of a fifth piece of tubing 7e can be connected to the outlet 32s of the second analyte sensor 38b and the other end can be connected to the inlet 31a of the air bubble eliminator 48. One end of a sixth piece of tubing 7f can be connected to the outlet 32a of the air bubble eliminator 48 and the other end can be connected to a second needle 8b which is can be inserted and secured proximate a second puncture site 27b on the individual and patient 28's arm 75 or leg 76. Additional valves 30 or roller clamps 85 for stopping or regulating the individual and patient's 28 blood 10 flow can be disposed before the inlets 31s and also after the outlets 32s of the analyte sensor devices 38a-b. The conditioning device 20, analyte sensor devices 38a-c, and pump 26 can be connected by wire or wirelessly to a computer 36 or other medical device having a monitor screen 37 and a keyboard 96, touch pad, and mouse.

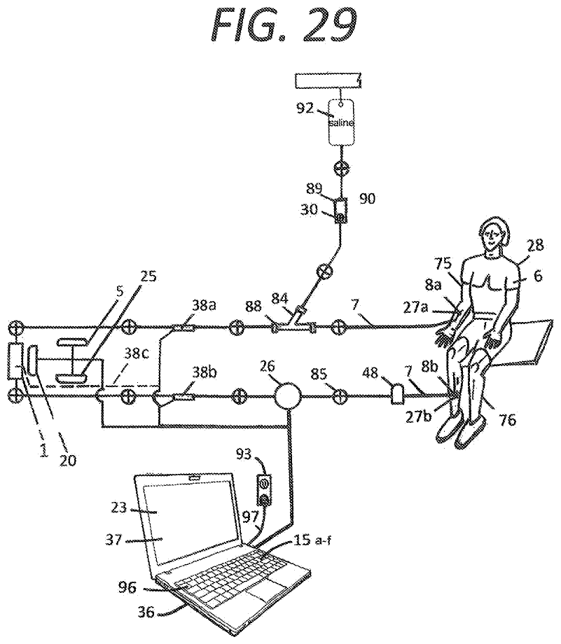

As shown in FIG. 29, the next simplest and alternative extracorporeal blood filtering device 33e, and related method, technique and process would be the same as that which has been just disclosed above, but can further include a first blood or blood portion conditioning device 5, hereinafter simply referred to as “conditioning device 5” or “first conditioning device 5” located between the first needle 8a and the inlet 31f of the filter device 1, and possibly also a third blood or blood portion conditioning device 25, hereinafter simply referred to as “conditioning device 25” or “third conditioning device 25,” located between the outlet 32f of the filter device 1 and the second needle 8b. The conditioning devices 5, 20 and 25, analyte sensor devices 38a-b, and pump 26 can be connected by wire or wirelessly to a computer 36 or other medical device having a monitor screen 37 and a keyboard 96, touch pad, and mouse.

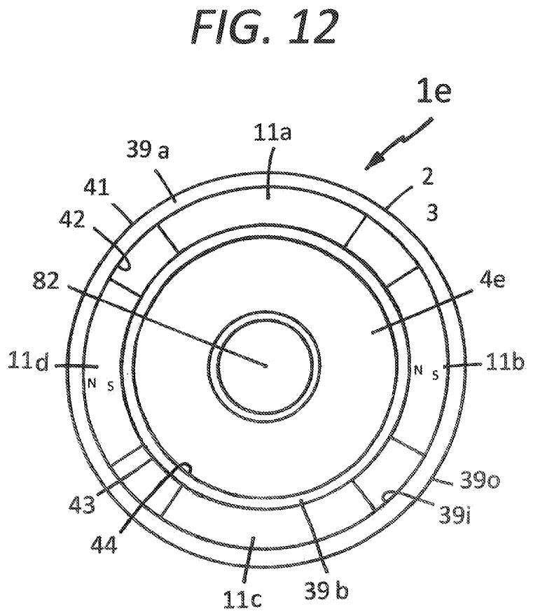

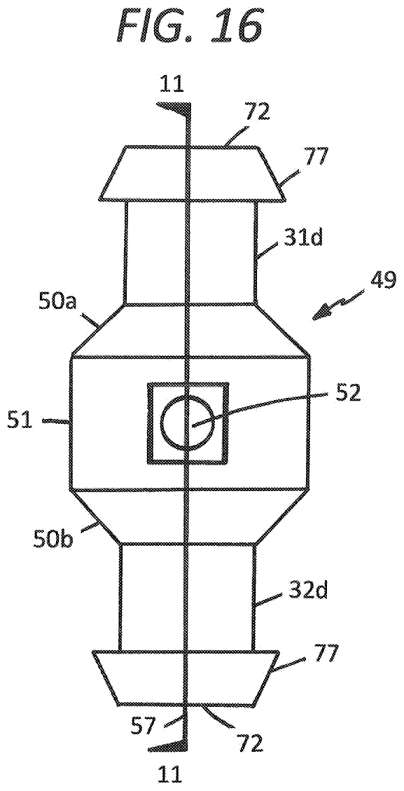

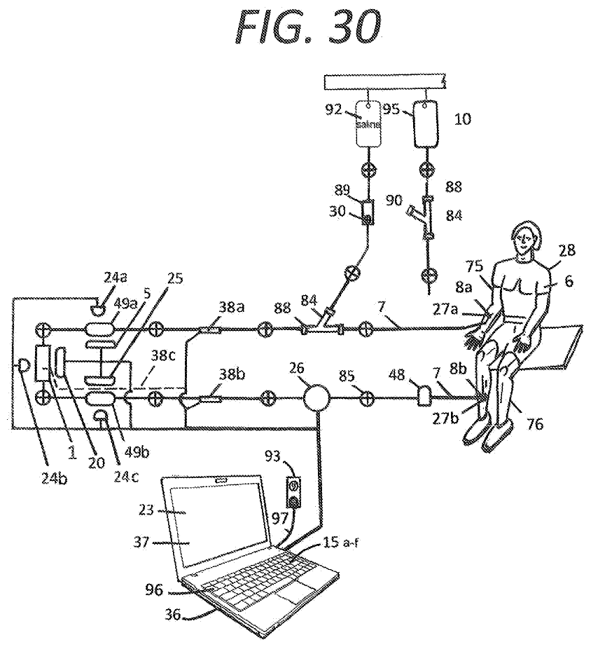

As shown in FIG. 30, the next simplest and alternative extracorporeal blood filtering device 33f, and related method, technique and process would be the same as that which has been just disclosed above, but can further include a first blood dispersion device 49a disposed between the first needle 8a and the inlet 31f of the filter device 1, and possibly also a second blood dispersion device 49b disposed between the outlet 32f of the filter device 1 and the inlet 31p of the blood pump 26. As shown in FIGS. 12 and 16, a blood dispersion device 49 can include an inlet 31d which leads to a first funnel portion 50a, a middle portion 51 having a relatively thin cross-section and a transparent window portion 52, and a second funnel portion 50b which leads to an outlet 32d. The thin cross-section can be configured to provide a gap for blood flow that is less than or equal to 50 microns wide, or alternatively less than or equal to 30 microns wide, or alternatively in the range between 6-15 microns wide, or alternatively in the range between 1.5-10 microns wide. In this regard, a gap in the range between 6-15 microns wide can permit the relatively unobstructed passage and also the observation of platelets, red blood cells, and most white blood cells in an individual and patient's 28 blood 10. The alternative extracorporeal blood filtering device 33d can include at least one optical monitor 24 disposed proximate to the window 52 of a blood dispersion device 49a-b. In this regard, a first optical monitor 24a can be disposed proximate to the window 52a portion of a first blood dispersion device 49a located between the first needle 8a and the inlet 31f of the filter device 1, a second optical monitor 24b can be disposed proximate to the filter device 1, and a third optical monitor 24c can be disposed proximate to a window 52b portion of a second blood dispersion device 49b located between the outlet 32f of the filter device 1 and the second needle 8b disposed at the second puncture site 27b. The optical monitor 24 can provide a microscopic view using a microscope, a stereo microscope, an electron microscope, a camera, or other optical device in order to provide for visual monitoring of an individual and patient's 28 blood portion and blood 10.

Accordingly, as shown in FIG. 30, a first needle 8a can inserted into a vein or artery of an individual and patient's 28 arm 75 and be secured proximate to the first puncture site 27a. One end of a first piece of tubing 7a can be connected to the first needle 8a and the other end can be connected to the inlet 31v of a valve 30 or roller clamp 85 which can stop or regulate the flow of an individual and patient's 28 blood 10. One end of a second piece of tubing 7b can be connected to the outlet 32v of the valve 30 or roller clamp 85 and the other end can be connected to the inlet 31s of the first analyte sensor 38a. One end of a third piece of tubing 7c can be connected to the outlet 32s of the first analyte sensor 38a and the other end can be connected to the inlet 32d of the first blood dispersion device 49a. One end of a fourth piece of tubing 7d can be connection to the outlet 32d of the first blood dispersion device 49a and the other end to the inlet 31f of a filter device 1. One end of a fifth piece of tubing 7e can be connected to the outlet 32f of the filter device 1 and the other end can be connected to the inlet 31d of the second blood dispersion device 49b. One end of a sixth piece of tubing 7d can be connected to the outlet 32d of the second blood dispersion device 49b and the other end can be connected to the inlet 31p of the blood pump 26. One end of a seventh piece of tubing 7f can be connected to the outlet 32b of the blood pump 26 and the other end can be connected to the inlet 31s of the second analyte sensor 38b. One end of an eighth piece of tubing 7e can be connected to the outlet 32s of the second analyte sensor 38b and the other end can be connected to the inlet 31a of the air bubble eliminator 48. One end of a ninth piece of tubing 7f can be connected to the outlet 32a of the air bubble eliminator 48 and the other end can be connected to a second needle 8b which is can be inserted and secured proximate a second puncture site 27b on the individual and patient 28's arm 75 or leg 76. Additional valves 30 or roller clamps 85 for stopping or regulating the individual and patient's 28 blood 10 flow can be located before the inlets 31d and also after the outlets 32d of the blood dispersion devices 49a-b. Further, in order to not unduly restrict the flow of an individual and patient's blood 10, a blood dispersion device 49a or 49b can alternatively be connected to tubing and two Y or T junctions configured to create a parallel line to the main line which is used to transport the blood portion or blood 10 of an individual and patient 28. The conditioning devices 5, 20, and 25, analyte sensors 38a-c, optical monitors 24a-c, and pump can be connected by wire or wirelessly to a computer 36 or other medical device having a monitor screen 37 and a keyboard 96, touch pad, and mouse.

It can be readily understood by a person of ordinary skill in the art, and in particular, a medical doctor, that the relative position and location where the valves 30 and/or roller clamps 85, tubing 7, analyte sensors 38, blood dispersion devices 49, filter device 1, blood pump 26, air bubble eliminator 48, optical monitors 24, blood conditioning devices 5, 20, and 25, wires 34 and computer 26 or other electronic medical device are disposed and located can possibly be modified and otherwise configured provided that such alternative configurations would not compromise the performance of an extracorporeal blood filtering device 33. For example, the order of the first analyte sensor 38a, the first valve 30 and/or roller clamp 85, and the first blood dispersion device 49a which are disposed between the first needle 8a and the filter device 1 could be changed. Likewise, the order of the second blood dispersion device 49b, the second analyte sensor 38b, and the pump 26 which are disposed between the filter device 1 and the second needle 8b could also be changed. Further, while some of the possible structures and configurations relating to the extracorporeal blood filtering devices 33a-f shown in FIGS. 25-30, and the methods, techniques and processes relating thereto have been discussed separately, it can be readily understood that depending on the condition of an individual and patient, and also the medical expertise and equipment which may be available, that some or all of the recited structures, configurations, methods, and technique can be selected and configured for use in various alternative partial combinations, or in a complete combination. In addition, the extracorporeal blood filtering devices 33a-f which have been disclosed in FIGS. 25-30 and discussed herein can be provided in various embodiments and in partial or complete combination in the form of prefabricated and ready-to-use medical kits which require little or no assembly. Moreover, depending on the condition of the individual and patient and also the medical expertise and equipment which is available, one or more of the device or structures which are typically included with the disclosed methods and techniques, such as a blood pump 26 or a filter device 1, can possibly be replaced and/or included within a more sophisticated medical device, such as one selected from the group of medical devices consisting of an apheresis device, a plasmapheresis device, an extracorporeal blood purification device, a hemofiltration device, a continuous veno-venous hemofiltration device, a hemodiafiltration device, an ultrafiltration device, and a hemodialysis or dialysis device.

### First Conditioning Device

As shown in FIG. 30, the extracorporeal blood filtering device 33f can include a first conditioning device 5 to introduce an electromagnetic field and gradient 45 that can impart an elevated electrical charge to or otherwise manipulate undesired matter 9 which is contained an individual or patient's 28 blood 10 after it has been removed from their body 6, but before it enters the inlet 31 of the filter device 1. For this purpose, the first conditioning device 5 can include at least one magnet device 11 such as a permanent magnet 12, a plurality of magnetic beads 13, or an electromagnet 14 which can be used to condition the undesired matter 9, which is typically negatively charged in the individual and patient's 28 blood 10, in order to facilitate its separation and removal. In this regard, a conditioning device 5 can include a variable control 15a for regulating the amount of voltage and current present in the electromagnet 14, and therefore the strength of the electromagnetic field that it will generate. Further, the conditioning device 5 can also include a variable control 15b for regulating the frequency and wavelength of the electromagnetic energy being generated. The conditioning device 5 can include a variable control 15c for selecting the type of waveform that will be produced, e.g., a square, saw, or sinusoidal wave form, and selecting whether the waveform is digital or analog. The conditioning device 5 can further include a variable control 15d for selecting and changing the magnetic poles of the electromagnet 14. In addition, the conditioning device 5 can include an AC versus DC variable control switch 15e for selecting either AC or DC as a source of power and/or for use in generating different types of waveforms. The conditioning device 5 can also include an on/off variable control switch 15f. The first conditioning device 5 can be disposed proximate to the narrow middle portion 51 of a first blood dispersion device 49a. The conditioning device 5 can also possibly include one or more gauges 22 and/or a conditioning device monitor screen 23 which can provide information and feedback with regards to the function of the variable controls 15a-f, and also regarding the nature and characteristics of the electromagnetic field and gradient 45 which is being produced. Alternatively, as shown in FIG. 30, the conditioning device 5 can be connected by wire 34 or wirelessly to a computer 36 or other medical device, and the one or more gauges 22 and monitor screens 23 can be displayed on at least one computer 36 monitor screen 37, and the variable controls 15a-f can be manipulated and controlled by using the computer's keyboard 96, touch pad or mouse.

### Second Conditioning Device

As shown in FIG. 30, the extracorporeal blood filtering device 33f can include a second conditioning device 20 which can include a magnet device 11 such as a permanent magnet 12 or an electromagnet 14 which is configured to provide an electromagnetic field and gradient 45 in order to help separate and remove undesired matter 9 such as graphene oxide, graphene hydroxide, magnetic graphene oxide, or other metals, pathogens, and toxins from blood 10. A second conditioning device 20 can be used whether or not a first conditioning device 5 has been used in order to impart an elevated electrical charge to or otherwise manipulate the undesired matter 9 present in the blood 10 of an individual and patient 28 and which typically has a negative electrical charge. Accordingly, the second conditioning device 20 is typically configured to provide an electromagnetic field and gradient 45 proximate to a filter 4 which is contained in the cavity 3 of a chamber 2 of a filter device 1 in order to manipulate the negatively charged undesired matter 9 so that it can be more effectively captured, mechanically trapped and/or adsorbed in the filter 4. In this regard, the second conditioning device 20 can be used to cause the undesired matter 9 to go into resonance, freeze, slow, or otherwise attract, entrain or direct its movement and so help the undesired matter 9 to be filtered and removed from the blood 10 of an individual and patient 28. However, when a first conditioning device 5 and/or third conditioning device 25 are not also being used, a second conditioning device 20 including permanent magnets 12, a plurality of magnetic beads 13, or electromagnets 14 can also be used to impart a neutral or other normalizing electromagnetic field and gradient 45 and positive or negative charge or to otherwise manipulate the red blood cells, white blood cells, platelets, and other components of normal human blood 10 which approximately matches the condition of healthy blood 10 and can possibly be used to correct and improve the health of an individual and patient's 28 blood 10. A second conditioning device 20 can include a variable control 15a for regulating the amount of voltage and current 15 present in the electromagnet 14, and therefore the strength of the electromagnetic field and gradient 45 that it will generate. Further, the second conditioning device 20 can also include a variable control 15b for regulating the frequency and wavelength of the electromagnetic energy being generated. The second conditioning device 20 can include a variable control 15c for selecting the type of waveform that will be produced, e.g., a square, saw, or sinusoidal wave form, and selecting whether the waveform is digital or analog. The second conditioning device 20 can further include a variable control 15d for selecting and changing the magnetic poles of the electromagnet 14. In addition, the second conditioning device 5 can include an AC versus DC variable control switch 15e for selecting either AC or DC as a source of power and/or for use in generating different types of waveforms. The second conditioning device 20 can also include an on/off variable control switch 15f. The second conditioning device 20 can also include one or more gauges 22 and/or at least one conditioning device monitor 23 which can provide information and feedback with regards to the function of the variable controls 15a-f, and also regarding the nature and characteristics of the electromagnetic field and gradient 45 which is being produced. Alternatively, the second conditioning device 20 can be connected by wire 24 or wirelessly to a computer 36 including a monitor screen 37, and the one or more gauges 22 and conditioning device monitor screen 23 can be displayed on a computer 36 monitor screen 37, and the variable controls 15a-f can be manipulated and controlled by using the computer's keyboard 96, touch pad or mouse.

### Third Conditioning Device

As shown in FIG. 30, the extracorporeal blood filtering device 33f can include a third conditioning device 25 which can include a magnet device 11 such as a permanent magnet 12 or an electromagnet 14 which is configured to provide an electromagnetic field and gradient 45 so as to impart a neutral or normalizing electrical charge or to otherwise manipulate the fluid or blood 10 of an individual and patient 28 after the fluid or blood 10 has left the outlet 32f of the filter device 1. In particular, a third conditioning device 25 can be used when a first conditioning device 5 has been used in order to impart an elevated electrical charge to or otherwise manipulate the electromagnetic field and gradient 45 which has been imparted to undesired matter 9 in an individual or patient's 28 blood 10, and/or when a second conditioning device 20 has been used to impart an electrical charge to or otherwise manipulate the electromagnetic field and gradient 45 proximate to a filter device 1 including a filter 4 included in in order to help separate and remove undesired matter 9 such as graphene oxide, graphene hydroxide, magnetic graphene oxide, or other metals, pathogens, and toxins from blood 10. In this regard, a third conditioning device 25 including permanent magnets 12, a plurality of magnetic beads 13, or electromagnets 14 can be used to impart either a neutral or a normalizing positive or negative charge and/or electromagnetic field and gradient 45 to the red blood cells, white blood cells, platelets, and other components of normal human blood 10 which approximately matches the condition of healthy blood 10, and in some cases can possibly correct and improve the status of a patient's blood 10. For example, the Ph of blood 10 could possibly be changed by the removal of negatively charged undesired matter 9, and also by the application of a normalizing electromagnetic filed and gradient 45 to an individual's blood 10. In this regard, a third conditioning device 25 can include a variable control 15a for regulating the amount of voltage and current 15 present in the electromagnet 14, and therefore the strength of the electromagnetic field that it will generate. Further, the third conditioning device 25 can also include a variable control 15b for regulating the frequency and wavelength of the electromagnetic energy being generated. The third conditioning device 25 can include a variable control 15c for selecting the type of waveform that will be produced, e.g., a square, saw, or sinusoidal wave form, and selecting whether the waveform is digital or analog. Further, the third conditioning device 25 can further include a variable control 15d for selecting and changing the magnetic poles of the electromagnet 14. In addition, the third conditioning device 25 can include an AC versus DC variable control switch 15f for selecting either AC or DC as a source of power and/or for use in generating different types of waveforms. The third conditioning device 25 can include an on/off variable control switch 15f. The third conditioning device 25 can also include one or more gauges 22 and at least one conditioning device monitor 23 which can provide information and feedback with regards to the function of the variable controls 15a-f and also regarding the nature and characteristics of the electromagnetic field and gradient 45 which is being produced. Alternatively, the third conditioning device 25 can be connected by wire 24 or wirelessly to a computer 36 including a monitor screen 37, and the one or more gauges 22 and conditioning device monitor screen 23 can be displayed on at least one computer 36 monitor screen 37, and the variable controls 15a-f can be manipulated and controlled by using the computer's keyboard 96, touch pad or mouse, as shown in FIG. 30. The third conditioning device 25 can be disposed proximate to the narrow middle portion 51 of a second blood dispersion device 49b. Alternatively, the first conditioning device 5, the second conditioning device 20, and the third conditioning device 25 can be configured as a single conditioning device 35 which has the capability of performing in partial or complete combination the desired or required functions of the first conditioning device 5, the second conditioning device 20, and the third conditioning device 25.

### Analyte Sensor

As shown in FIG. 30, an electronic analyte sensor device 38 can be further included and used to identify and communicate the presence and levels of targeted undesired matter 9 which is possibly contained in the blood 10 of an individual and patient 28. In this regard, a first analyte sensor device 38a can be disposed between the location of the first needle 8a at the first puncture site 27a, and the inlet 31f of the filter device 1 in order to determine the presence and levels of undesired matter 9 in the unfiltered blood 10 of an individual and patient 28. In addition, a second analyte sensor device 38b can be disposed between the outlet 32f of the filter device 1 and the second needle 8b located at the second puncture site 27b in order to determine the presence and levels of undesired matter 9 in the filtered blood 10 of an individual and patient 28. For research purposes, a third analyte sensor device 38c shown using dashed lines can be disposed inside or proximate to a filter device 1 to monitor the presence and level of undesired matter 9 which is contained in the blood 10 of an individual and patient 28 while the blood 10 is being filtered by the filter device 1 and is also then possibly under the influence of the second conditioning device 20. The analyte sensor devices 38a, 38b can be connected by wire 34 or wirelessly to a computer 36 including a monitor screen 37, keyboard 96, touch pad, and mouse, or other medical device which includes controls for operating the analyte sensor devices 38a-c, and which uses a software application for comparing and monitoring the presence and levels of undesired matter 9 found by analyte sensors 38a-c in an individual and patient's blood 10.

### Optical Monitor Device

As shown in FIG. 30, an optical monitor 24 such as a camera device including a macro lens, a microscope, a stereo microscope, an electron microscope or other form of optical monitoring equipment can also be included and used to visually observe the content, structure, and behavior of undesired matter 9, red blood cells, white blood cells, platelets, and plasma in an individual and patient's 28 blood 10. For example, a first optical monitor 24a can be disposed between the location of the first needle 8a and puncture site 27a and the inlet 31f of the filter device 1 and proximate to a window 52 in the middle portion 51 of a first blood dispersion device 49a which is disposed in about the same location in order to observe and monitor the blood 10 of an individual and patient 28 before it is filtered by the filter device 1 and is then also possibly under the influence of a first conditioning device 5. A third optical monitor 24c can be disposed between the outlet 32f of the filter device and the location of the second needle 8b and puncture site 27b and proximate to a window 52 in the middle portion 51 of a second blood dispersion device 49b which is disposed in about the same location in order to observe and monitor the blood 10 of an individual and patient 28 after it has been filtered by the filter device 1 and is then also possibly under the influence of a third conditioning device 25. For research purposes, a second optical monitor 24b can possibly be disposed inside or proximate to a filter device 1 in order to observe and monitor the behavior of undesired matter 9 which is contained in the blood 10 of an individual and patient 28 while the blood 10 is being filtered by the filter device 1 and is also then possibly under the influence of a second conditioning device 20. In this regard, an optical monitor 24 can be connected by wires 34 or alternatively wirelessly to a computer 36 including a monitor screen 37, keyboard 96, touch pad, and mouse, or other medical device which includes at least one monitor screen 37 and one or more views for monitoring the blood 10 of an individual and patient 28.

### Computer Monitor and Control Device

As shown in FIG. 30, a computer 36 including a monitor screen 37, keyboard 96, touch pad, mouse, and other means for providing output such as a printer, a computer network, or other medical device can be placed in electronic communication by wire 34 or wirelessly with a plurality of optical monitors 24, and/or analyte sensor devices 38 so that blood 10 of an individual or patient 28 can be monitored in a plurality of locations, such as before the blood 10 enters the inlet 31f of a filter device 1, while the blood 10 is contained within the filter device 1, and after the blood 10 has left the outlet 32f of the filter device 1. Further, a computer 26 including a monitor screen 37, keyboard 96, touch pad, mouse, and other means for providing output such as a printer, a computer network, or other medical device can be placed in electronic communication by wire 34 or wirelessly with at least one of the first conditioning device 5, the second conditioning device 20, and the third conditioning device 25 in order to monitor and control the effects of the conditioning devices 5, 20, and 25 upon the undesired matter 9 and other structures contained in the blood portion or blood 10 of an individual or patient 28. In this regard, the computer 36 including a monitor screen 37, keyboard 96, touch pad, mouse, and other means for providing output such as a printer, a computer network, or other medical device can control and monitor the conditioning devices 5, 20, and 25, and this can include a variable control 15a for regulating the amount of voltage and current present in the electromagnet 14, and therefore the strength of the electromagnetic field that it will generate, a variable control 15b for regulating the frequency and wavelength of the electromagnetic energy being generated, a variable control 15c for selecting the type of waveform that will be produced, e.g., a square, saw, or sinusoidal wave form, and selecting whether the waveform is digital or analog, a variable control 15d for selecting and changing the magnetic poles of the electromagnet 14, a variable control switch 15e for selecting either AC or DC as a source of power and/or for use in generating different types of waveforms, and also an on/off variable control switch 15f. The computer 36 including a monitor screen 37, keyboard 96, touch pad, mouse, and other means for providing output such as a printer, or other medical device can provide information and feedback with regards to the function of the variable controls 15a-f and also regarding the nature and characteristics of the electromagnetic field and gradient 45 which is being produced. The computer 36 including a monitor screen 37, keyboard 96, touch pad, mouse, and other means for providing output such as a printer, computer network, or other medical device can be connected by wire or wirelessly to one or more magnetic devices 11, optical monitors 24, pump devices 26, analyte sensor devices 38, and/or to a medical device selected from the group of medical devices consisting of an apheresis device, a plasmapheresis device, an extracorporeal blood purification device, a hemofiltration device, a continuous veno-venous hemofiltration device, a hemodiafiltration device, an ultrafiltration device, and a hemodialysis or dialysis device.

### Filter Device

Many different kinds of devices and filters have been developed in order to remove and/or separate the four main constituent portions of human blood, but also to remove bacterial and viral pathogens, toxins, and other undesired matter from fluids, portions of blood, and blood. In medical practice, it is also known to add to human blood analgesics, chemical compositions, drugs, electrolytes, or other forms of matter, e.g., chemical markers, radioactive substances, and chelators. The latter are sometimes used in chelation therapy, which sometimes uses CaNa₂EDTA (Calcium Disodium Ethylenediaminetetraacetate) to remove heavy metals from the bloodstream so that they can be excreted in order to correct an individual's blood and restore heath. Besides the metals aluminum and mercury which have been used as adjuvants and/or preservatives in some vaccines, other heavy metals such as antimony, arsenic, beryllium, cadmium, hexavalent chromium, cobalt, copper, lead, manganese, nickel, silver, and zinc in large enough quantities be toxic. Electrically conductive metals, and materials of the graphene family such as graphene oxide, graphene hydroxide, magnetic graphene oxide, but also many pathological or toxic forms of matter, diseased cells, cellular debris, and waste matter will typically exhibit a negative charge when present in a fluid, blood portion or blood 10 of an individual and patient 28. As a result, undesired matter 9, bacteria, viruses, infected cells, cellular debris, waste products, and pathological or toxic forms of matter present in human blood 10 can be removed with the use of a magnetic device 11 which can provide an electromagnetic field and gradient 45 and filters 4 which incorporate materials which are or can be positively charged. For this reason, a magnetic device 11 which can be a permanent magnet device 12, a plurality of magnetic beads 13, or an electromagnet device 14 can be used to help separate and remove undesired matter 9 from a fluid, blood portion or blood 10 of an individual and patient 28, as will be discussed in greater detail below.

Conventional filter devices and filters are typically designed, configured and engineered to work and be effective in removing substances and matter from aqueous substances, organic substances, or a combination of both. For use in association with aqueous substances filters are typically made of hydrophilic materials such as cellulose/cellulose acetate (CA), or polyethersulfone (PES), whereas hydrophobic materials such as polypropylene (PP) or polytetrafluoroethylene (PTFE) are more commonly used to filter organic matter, but depending of the matter which is to be separated and the circumstances, one, the other, or combinations of different filters can be used to mixtures of aqueous and organic matter. Examples of non-electrically charged filters made by the 3M company of St. Paul, Minnesota include the 3M™ LifeASSURE™ line of PDA filters capsules and cartridges which feature a 0.2 micron/200 nm, or a 0.1 micron/100 nm particle rated membrane and include a double-asymmetric membrane layer filter construction. For more information, see the pdf document which can be obtained from the 3M website: https://multimedia.3m.com/mws/media/14444440/3m-lifeassure-pda.pdf. Other manufacturers of non-electrically charged filters include B. Braun Medical, Inc. of Bethlehem, Pennsylvania and B. Braun Melsunger AG, located in Melsungen, Germany, and also Corbetter Filtration Equipment Co., LTD, of Hangzhou, China. However, the 0.1 micron or 0.2 micron particle rated membrane size of the aforementioned 3M LifeASSURE™ line of PDA filters is too small to permit blood platelets which are commonly 1.5-4 microns, red blood cells which are commonly 6-9 microns in diameter, and most white blood cells which are commonly 7-15 microns to pass through their filter media, and so such a filter would trap just about everything normally contained in human blood except for plasma. Further, the size of graphene oxide, graphene hydroxide, or magnetic graphene oxide, and like foreign undesired matter 9 can be on the order of only about 1 nm in thickness, and commonly between 1-1000 nanometers or more in length and width, and so small pieces could possibly be able to pass through the 3M LifeASSURE™ filter membrane. Graphene oxide can also form in generally planar sheets, and these can also possibly congregate, aggregate, and self-assemble to form large clumps of undesired matter 9.

Examples of conventional electrically charged filters include the 3M™ Zeta Plus™ ZB line and series filter capsules and cartridges. The ZB media incorporated into these 3M filters imparts a positive charge to the fiber matrix of the filter which can attract negatively charged matter, and as result the filters can use both mechanical entrapment and electrokinetic adsorption to remove undesired matter. In this regard, the ZB media and related filters are available in six grades which exhibit different levels of positive charge, and they also available in many different sizes. Further, many different filter particle rated membrane sizes are available in the range between 0.1-4 microns or 100-4000 nm. For more information, see the 3M website: https://www.3 m.com/3M/en_US/p/d/b40070867/ and 3M literature 70-0203-0883-2. Another manufacturer of electrically charged filters is B. Braun Medical, Inc. of Bethlehem, Pennsylvania and B. Braun Melsunger AG, located in Melsungen, Germany which makes Intrapur® Plus filters with positively charged membranes. However, the 0.1-0.4 micron or 100-400 nanometer particle rated membrane size of the aforementioned 3M™ Zeta Plus™ ZB line and series filter capsules and cartridges is also too small to permit blood platelets which are commonly 1.5-4 microns, red blood cells which are commonly 6-9 microns in diameter, and white blood cells which are commonly 7-15 microns to pass through the filter media, and so such a filter would trap almost everything contained in human blood except for plasma. Further, the size of graphene oxide, graphene hydroxide, or magnetic graphene oxide, and like undesired matter 9 can be on the order of only about 1 nm in thickness, and commonly between 1-1000 or more nanometers in length and width, and so small pieces could possibly be able to pass through the filter membrane. Graphene oxide can also form in generally planar sheets, and these can also congregate, aggregate, and self-assemble to form large clumps of undesired matter 9.

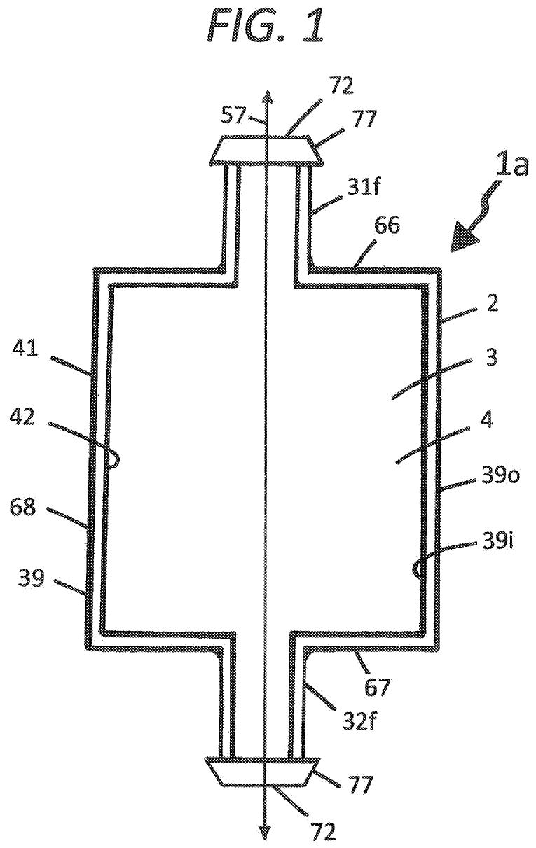

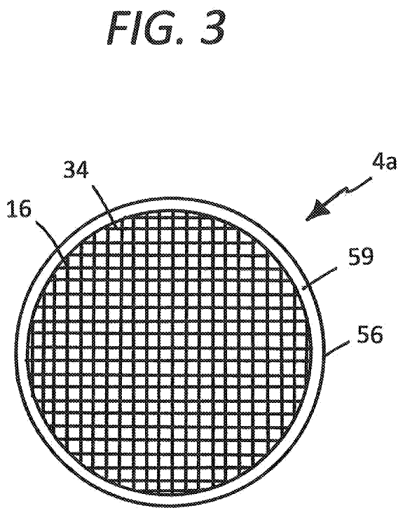

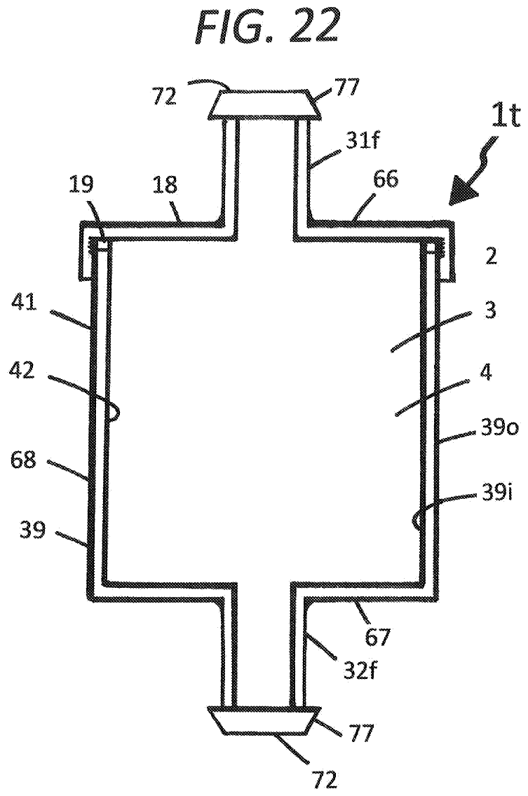

FIG. 1 is a side cross sectional view of a filter device 1a which includes a wall 39 which can be made of a biocompatible material such as a medical grade metal, glass, or thermoplastic material, e.g., polyamides or nylon, polyethylene glycol, polyvinyl alcohol, polyethyleneimine, polyvinylamine, polyacrylates, polyesters, polycarbonates, polystyrenes, polypropylene, polydimethylsiloxane or silicone, ABS, urethanes, nylon, PTFE, and a thermoplastic elastomer such as Santoprene. The wall 39 include an outside wall surface 39o which is on the exterior 41 of the filter device 1a, and an inside wall surface 39i which is on the interior 42 of the filter device 1a. The filter device 1a can include an integral or removable inlet 31f and outlet 32f. The inlet 31f and outlet 32f can include barbs, threads, luer 94, or other types of male and female or hermathrodite structures for connecting and coupling the filter device 1a to tubes 7, and/or other structures or devices. The filter device 1a can have a top side 66, a bottom side 67, and one or more lateral side(s) 68 which can be straight or curved depending on whether the side 68 portion is configured to be cylindrical in shape and then possibly also have a radius oriented about a central axis 57, or instead be configured in a different geometric shape. As shown in FIG. 1, the filter device 1a is configured with the inlet 31f and outlet 32f being in alignment about a central axis 57 which can enhance the flow through of a liquid, blood portion and blood 10. The structure of the filter device 1a defines a chamber 2 which includes a cavity 3 which includes a void space when empty and which has a volume. The filter device 1a further includes a filter 4, such as one of the filters 4a-e shown in FIGS. 3, 4, 5, 7, 8, which is used to help separate and remove undesired matter 9 such as metals, graphene oxide, graphene hydroxide, magnetic graphene oxide, pathogens, and toxins from an individual and patient's 28 blood portion and blood 10. When a non-electrically charged and/or a positively electrically charged filter 4 is being used, it can be helpful to dispose a magnetic device 11 which can be a permanent magnet 12, or an electromagnet 14 which produces a magnetic field and gradient 45 that can impart an elevated electrical charge, and typically a positive charge proximate to the filter 4. In this regard, a filter 4 which already includes an internal structure and/or media which is and/or can be electrically charged is typically selected for use. In this regard, the filter structure and/or media is typically positively electrically charged. As shown in FIG. 1, the filter 4 can be permanently sealed and contained within the chamber 2 of the filter device 1a, or alternatively the filter 4 can be configured as a removable cartridge 55 and be replaced when a filter device it includes a screw-off or otherwise removable top portion 18 and seal 19, as shown in FIG. 22.

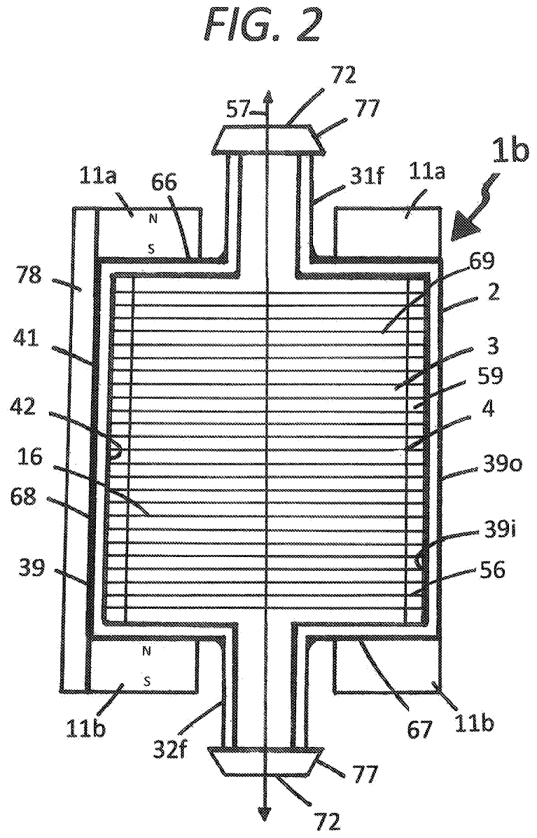

FIG. 2 is a side cross-sectional view of an alternative embodiment of a filter device 1b which includes a wall 39 which can be made of a biocompatible material which can be a medical grade metal, glass, or thermoplastic material, e.g., polyamides or nylon, polyethylene glycol, polyvinyl alcohol, polyethyleneimine, polyvinylamine, polyacrylates, polyesters, polycarbonates, polystyrenes, polypropylene, polydimethylsiloxane or silicone, ABS, urethanes, nylon, PTFE, and a thermoplastic elastomer such as Santoprene. The wall 39 includes an outside wall surface 39o which is on the exterior 41 of the filter device 1b, and an inside wall surface 39i which is on the interior 42 of the filter device 1b. The filter device 1b can include an integral or removable inlet 31f and outlet 32f. The inlet 31f and outlet 32f can include barbs, threads, luer 94, or other types of male and female or hermathrodite structures for connecting and coupling the filter device 1b to tubes 7, and/or other structures or devices. The filter device 1b can have a top side 66, a bottom side 67, and one or more lateral side(s) 68 which can be curved or straight depending on whether it is cylindrical in shape and then have a radius which is oriented about a central axis 57, or is configured in a different geometric shape. As shown in FIG. 2, the filter device 1b is configured with the inlet 31f and outlet 32f being in alignment which can enhance the flow through of a liquid, blood portion and blood 10. The structure of the filter device 1b defines a chamber 2 which includes a cavity 3 which includes a void space when empty and which has a volume. As shown in FIG. 2, the filter 4 can be permanently sealed and contained within the chamber 2 of the filter device 1b, or alternatively the filter 4 can be configured as a removable cartridge 55 and be replaced when a filter device it includes a screw-off or otherwise removable top portion 18 and seal 19, as shown in FIG. 22.

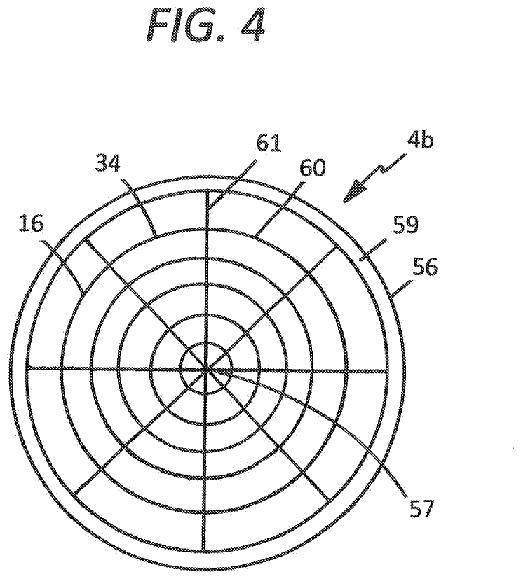

The filter device 1b includes a filter 4 such as filter 4a-b shown in FIGS. 3-4 which is used to help separate and remove undesired matter 9 such as metals, graphene oxide, graphene hydroxide, magnetic graphene oxide, pathogens, and toxins from an individual and patient's 28 blood 10. When a non-electrically charged filter 4 is being used, it can be helpful to use a permanent magnet 12, a plurality of magnetic beads 13, or an electromagnet 14 which produces a magnetic field and gradient 45 that can impart a positive charge proximate to the filter 4. However, a filter 4 that includes an internal structure and/or media which is or can be electrically charged is typically selected for use. In this regard, the filter structure and/or media can be and is typically positively electrically charged. As shown, e.g., in FIGS. 3-4 a filter 4a-b can include a filter layer 56 including a screen 16 structure which is made of an electrically conductive metal, ceramic, or other composite material. As shown in FIG. 2, a plurality of twenty five filter layers 56 can be disposed, nested, or stacked next to one another and include a border 59, and the filter layers 56 can be orientated perpendicular to a central axis 57 associated with the configuration of the inlet 31f and outlet 32f and direction of flow of a liquid, blood portion and blood 10 to form a complex matrix or maze 69. As shown in FIG. 2, one or more magnet devices 11a-b can be removably or permanently secured proximate to the wall 39, and in particular, on the exterior 41 near the outside wall surface 39o of the filter device 1b. In this regard, the magnet devices 11a-b can be permanent magnets 12, a plurality of magnetic beads 13, or electromagnets 14. As shown in FIG. 2, when a plurality of magnet devices 11a-b are used, the magnet devices 11 are typically orientated so that their north and south poles are configured to face the oppositive poles of the magnetic device 11 which is disposed nearby, that is, the south pole S of magnet device 11a is disposed opposite to the north pole N of magnet device 11b. In other words, a first magnet device 11a having a first positive pole and first negative pole is typically configured such that the first positive pole is disposed proximate to the second negative pole of the second magnet device 11b, and/or the first negative pole is disposed proximate to the second positive pole. In FIG. 2, the magnet devices 11 are shown disposed proximate to the top side 66 and bottom side 67 of the filter device 1b. As shown in FIG. 2, the magnetic devices 11a and 11b can also be electrically connected by a conductive bridge 78.

FIG. 3 is a top view of a layer 56 of screen 16 for use in a filter 4a. A screen 16 can be configured in two superimposed, overlapping and joined perpendicular partial-layers or by two or more woven partial-layers of metal wire 34 or other electrically conductive material. A screen 16 can also be configured by knitting, or can alternatively be configured and made by 3D printing or other manufacturing processes, and can also be referred to as a mesh, net, or web. The layer 56 of screen 16 includes a border 59 which provides for a mating surface, seat, and/or seal between adjoining layers 56. The layer 56 can be an integral and permanent part of a filter device 1 such as filter device 1b shown in FIG. 2, or alternatively can be part of a removable and replaceable cartridge 55 when a filter device 1t includes a removable top portion 18 and seal 19, as shown in FIG. 22. While the drawing figures show various embodiments of screens 16, meshes, nets or webs for use in filters 4 which can be easily seen, it can and should be readily understood that regarding any or all of the different embodiments of a filter 4 the size of the screen 16, mesh, net, or web can typically be equal to or less than 2 mm or equal to or less than 1000 microns, or equal to or less than 270 microns, or equal to or less than 160 microns, or equal to or less than 50 microns, or equal to or less than 30 microns, or equal to 15 microns, but will typically be greater than the range between 1-15 microns when the passage of plasma, platelets, red blood cells, and white blood cells in a blood portion and blood 10 is desired or required. However, when filtering blood plasma containing undesired matter 9, e.g., a member of the graphene family such as graphene oxide, or a different pathogen or toxin that is typically smaller in size than 15 microns, the size of the particles which the filter 4 can effectively filter can be configured to be equal to or less than 15 microns, or be equal to or less than 2 microns, or be equal to or less than 1 micron, or be equal to or less than 0.2 microns, or be equal to or less than 0.1 micron and then be in the range between 1-100 nanometers.

FIG. 4 is a top view of an alternative embodiment of a layer 56 including a web-like screen 16 for use in a filter 4b. As shown, an alternative layer 56 can include a plurality of concentrically orientated rings 60 of metal wire or other electrically conductive material which are spaced about a central axis 57 and can be connected together by one or more cross braces 61. The alternative layer 56 can also be configured by superimposing wires 34, weaving, knitting, or be configured and made by 3D printing or other manufacturing processes, and can also be referred to as a mesh, net, or web. In FIG. 4, the alternative layer 56 of screen 16 includes a border 59 which provides for a mating surface, seat, and/or seal between the filter 4b and an inside wall portion 39i of the filter device 1b. The alternative layer 56 can be an integral and permanent part of a sealed filter device 1 such as filter device 1b shown in FIG. 2, or alternatively can be part of a replaceable cartridge 55 when a filter device It includes a removable top portion 18 and seal 19, as shown in FIG. 22.

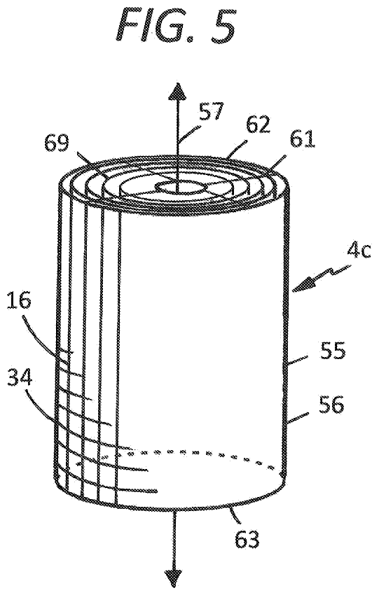

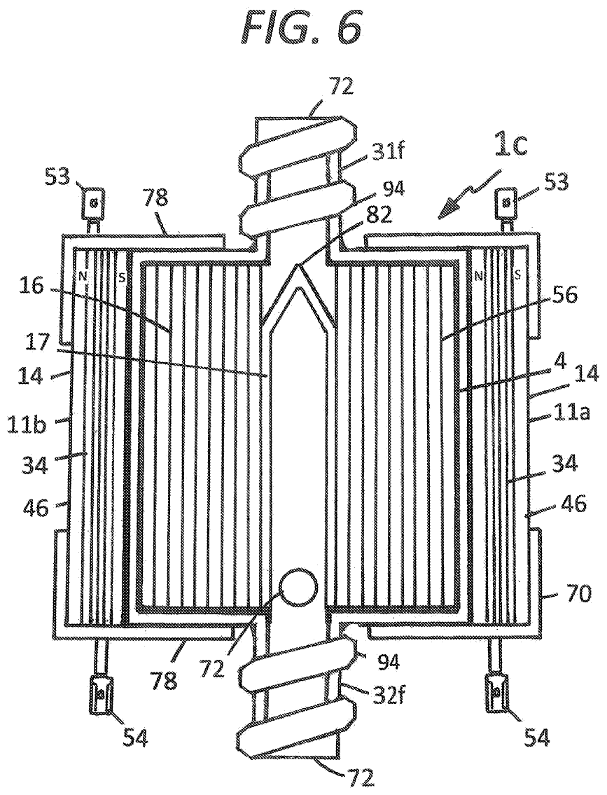

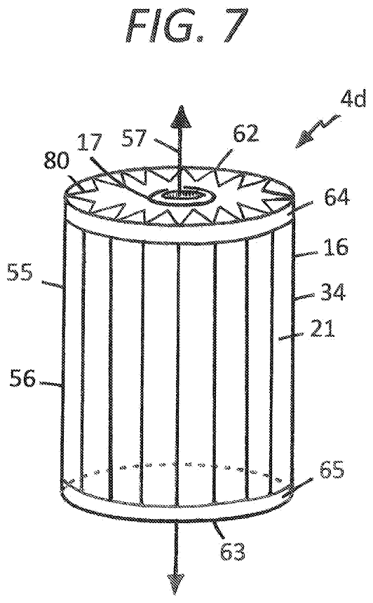

FIG. 5 is a top perspective view of the alternative filter 4c which can include a plurality of circular and concentrically orientated layers 56 of metal wire or other electrically conductive material. The layers 56 are spaced around and about a central axis 57 and can be connected together by one or more cross braces 61. The screen 16 portions of the filter 4c can be configured and made by superimposing wires 34, weaving, knitting, or by using 3D printing or other manufacturing processes, and can also be referred to as a mesh, a net, or a web. Alternatively, the top end 62 and bottom end 63 of the layers 56 of screen 16 can also include a top seal 64 and bottom seal 65, as shown with the filter 4d shown in FIG. 7, which can provide a mating surface, seat, and/or seal between the filter 4c and an inside wall portion 39i of a filter device 1. In an alternative embodiment, the screens 16 used in filter 4c can further include or be connected to an additional filter media 21 for mechanically trapping and/or adsorbing undesired matter 9 from a liquid, blood portion and blood 10. The filter 4c can be an integral and permanent part of a sealed filter device 1 such as filter device 1c shown in FIG. 6, or can be a removable and replaceable cartridge 55 when a filter device it includes a removable top portion 18 and seal 19, as shown in FIG. 22.

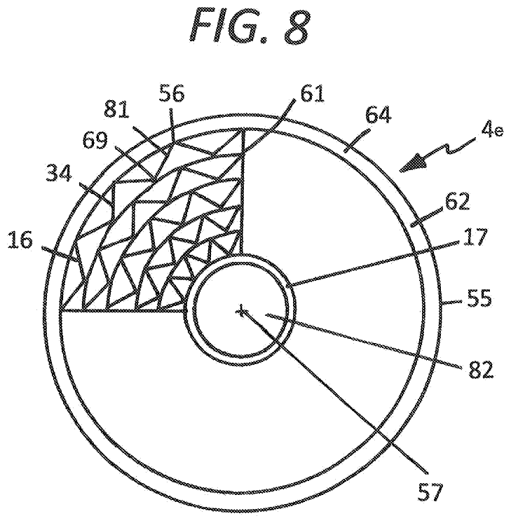

FIG. 6 is a side view of an alternative filter device 1c which includes a wall 39 which can be made of a biocompatible material such as a medical grade metal, glass, or thermoplastic material, e.g., polyamides or nylon, polyethylene glycol, polyvinyl alcohol, polyethyleneimine, polyvinylamine, polyacrylates, polyesters, polycarbonates, polystyrenes, polypropylene, polydimethylsiloxane or silicone, ABS, urethanes, nylon, PTFE, and a thermoplastic elastomer such as Santoprene. As shown and previously discussed with regards to FIG. 1, the wall 39 includes an outside wall surface 39o which is on the exterior 41 of the filter device 1c, and an inside wall surface 39i which is on the interior 42 of the filter device 1c. The filter device 1c, further includes a ledge 70 and/or conductive bridge 78 which projects from the outside wall surface 39o of the chamber 2 on the exterior 41 of the filter device 1c which can serve as a support and/or bracket for securing removable magnetic devices 11a-b. Accordingly, the magnetic devices 11a-b can be placed in close proximity to a filter 4, such as filters 4a-e. The filter device 1c can include an integral or removable inlet 31f and outlet 32f. The inlet 31f and outlet 32f can include barbs, threads, luer 94, or other types of male and female or hermathrodite structures for connecting and coupling the filter device 1b to tubes 7, and/or other structures or devices. In FIG. 6, the inlet 31f and outlet 32f portions include male luer 94 style connectors for coupling with complimentary female luer style connectors which can be used to removably secure pieces of tubing 7 to the filter device 1c. The filter device 1c can have a top side 66, a bottom side 67, and one or more lateral side(s) 68 depending on whether the side 68 portion is configured as being circular in shape and has a radius oriented about a central axis 57, or other geometric shape. As shown in FIG. 6, the filter device 1c is configured with the inlet 31f and outlet 32f being in alignment which can enhance the flow through of a liquid, blood portion and blood 10. The structure of the filter device 1c defines a chamber 2 which includes a cavity 3 including a void space when empty and which has a volume. A filter 4, such as filter 4c shown in FIG. 5, filter 4d shown in FIG. 7, or filter 4e shown in FIG. 8 can be permanently sealed and contained within the chamber 2 of a filter device 1c, or alternatively can be configured as a removable cartridge 55 and be removed and replaced when a filter device it includes a screw-off or otherwise removable top portion 18 and seal 19, as shown in FIG. 22. FIG. 6 shows a filter 4 which generally resembles the filter 4c shown in FIG. 5, but which includes ten layers 56 that each include a screen 16 which are configured to be disposed and oriented parallel to a central axis 57 of the filter device 1c. The location of a central axis 57 has already been shown relative to the location of the inlet 31f and outlet 32f of filter device 1b shown in FIG. 2, and it is similarly located with respect to filter device 1c shown in FIG. 6.

The filter device 1c can include a filter 4c, 4d, or 4e which is used to help separate and remove undesired matter 9 such as metals, graphene oxide, graphene hydroxide, magnetic graphene oxide, pathogens, and toxins from an individual and patient's 28 blood portion and blood 10. When a non-electrically charged filter 4 is being used, it can be helpful to use a magnetic device 11a-b, such as a permanent magnet 12, a plurality of magnetic beads 13, or an electromagnet 14 which produces an electromagnetic field and gradient 45 that imparts a positive charge proximate to the filter 4. However, a filter 4 such as 4c, 4d, or 4e which includes an internal structure and/or media which is or can be electrically charged is typically selected for use. In this regard, the filter structure and/or media is typically positively electrically charged. As shown in FIGS. 5, 7, and 8, a filter 4c, 4d, or 4e can include a plurality of circular concentrically orientated layers 56 or other geometric shapes including but not limited to a convoluted structure 80 or a zig-zag structure 81 and can include screens 16 made of metal wire 34 or other electrically conductive material which are spaced around and about a central axis 57 and which can possibly be connected together by one or more cross braces 61 or other means for making a permanent or removable connection. The screens 16 can also be configured by superimposing wires 34, weaving, knitting, or be configured and made by 3D printing or other manufacturing processes, and can also be referred to as a mesh, net, or web, and be made of an electrically conductive metal, ceramic, or other composite material. In an alternative embodiment, the screens 16 used in a filter 4c, 4d, or 4e can further include or be connected to an additional filter media 21 for mechanically trapping and/or adsorbing undesired matter 9 from a liquid, blood portion and blood 10. The alternative filter 4c, 4d, or 4e can be an integral component and be permanently sealed and contained within the chamber 2 of a filter device 1 such as filter device 1c shown in FIG. 6, or can alternatively be a removable and replaceable cartridge 55 and be replaced when a filter device 1 such as filter device it also includes a screw-off or otherwise removable top portion 18 and seal 19, as shown in FIG. 22.

As shown in FIGS. 5, 7 and 8, a plurality of filter layers 56 can be configured and disposed next to one another and the filter layers 56 can be orientated parallel to a central axis 57 associated with the configuration of the inlet 31f and outlet 32f, and also the direction of flow of a liquid, blood portion and blood 10 and then form a complex matrix or maze 69. As shown in FIGS. 5, 7, and 8, a filter 4c, 4d, or 4e can include a plurality of circular and/or concentrically orientated filter layers 56 including screens 16 of metal wire 34 or other electrically conductive material which are spaced about a central axis 57 and a connected together by one or more cross braces 61 or other means for making a connection, and can also possibly include a mechanically, chemically, and/or adhesively connected top seal 64 on the top end 62 and bottom seal 65 on the bottom end 63 as shown in FIGS. 7-8. Further, one or more magnet devices 11a-b can be removably or permanently secured proximate to the wall 39, and in particular, near the outside wall surface 39o of the filter device 1c. In this regard, the magnet devices 11a-b can be permanent magnets 12, a plurality of magnetic beads 13, or electromagnets 14. As shown in FIG. 6, when a plurality of magnet devices 11a-b are used with a filter device 1c which includes a filter 4c, 4d, or 4e, the magnet devices a-b are typically orientated so that their north and south poles are configured to face the oppositive poles of the magnetic device 11 which is disposed nearby, that is, the south pole S of magnet device 11a is disposed opposite to the north pole N of magnet device 11b. In other words, a first magnet device 11a having a first positive pole and first negative pole is typically configured such that the first positive pole is disposed proximate to the second negative pole of the second magnet device 11b, and/or the first negative pole is disposed proximate to the second positive pole. In FIG. 6, the magnet devices 11a-b are electromagnets 14 which are disposed proximate to the lateral side(s) 68 of the filter device 1c, and include conductive wire 34 disposed around a conductive core 46, and also male lead 53 and female lead 54 connectors.

FIG. 7 is a top perspective view of an alternative filter 4d which can include a plurality of convoluted 80 layers 56 including screens 16 of metal wire 34 or other electrically conductive material which project outwards around and about a central axis 57. The layers 56 can be configured so as to nest close to one another and be disposed inside of one another. The layers 56 can be connected together by one or more cross braces 61 and/or at the top end 62 and bottom end 63 by a top seal 64 and a bottom seal 65. The screens 16 can be configured by superimposing wires 34, weaving, knitting, or be configured and made by 3D printing or other manufacturing processes and can also be referred to as a mesh, net, or web. In FIG. 7, the top ends 62 and bottom ends 63 of the layers 56 of screen 16 include a top seal 64 and bottom seal 65 which provides for a mating surface, seat, and/or seal between the filter 4d and an inside wall surface 39i of a filter device 1 such as filter device 1c shown in FIG. 6. In an alternative embodiment, the screens 16 used in filter 4d can further include or be connected to an additional filter media 21 for mechanically trapping and/or adsorbing undesired matter 9 from a liquid, blood portion and blood 10. The alternative filter 4d can be an integral component and be permanently sealed and contained within the chamber 2 of a filter device 1 such as filter device 1c shown in FIG. 6, or can alternatively be a removable and replaceable cartridge 55 and be replaced when a filter device it also includes a screw-off or otherwise removable top portion 18 and seal 19 as shown in FIG. 22.

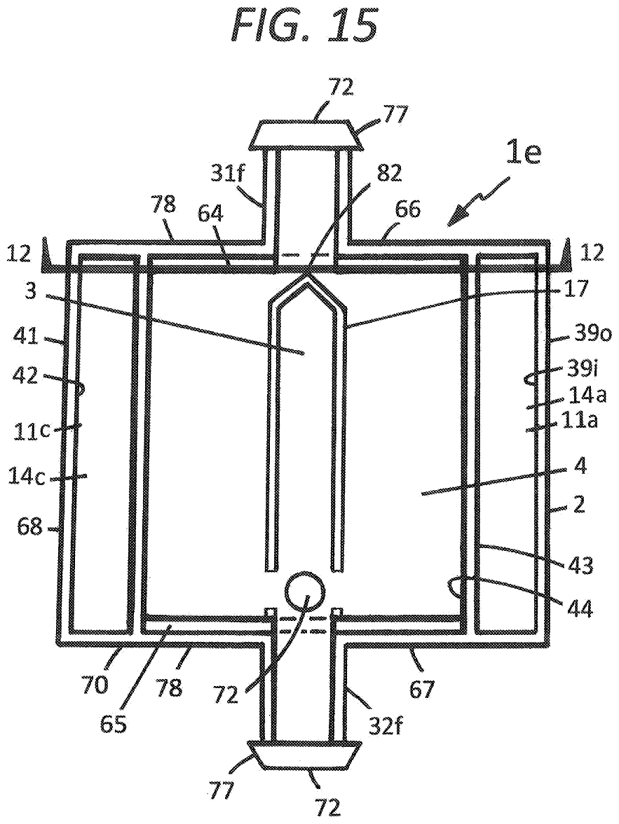

FIG. 8 is a top perspective view of an alternative filter 4e which can include a zig-zag structure 81 in a plurality of layers 56 including screens 16 of metal wire 34 or other electrically conductive material which project outwards around and about a central axis 57. The layers 56 can possibly be connected together by one or more cross braces 61, and/or at the top end 62 and bottom end 63 by a top seal 64 and a bottom seal 65. The layers 56 can be configured so as to nest close to one another and be disposed inside of one another. The screens 16 can also be configured by superimposing wires 34, weaving, knitting, and/or be configured and made by 3D printing or other manufacturing processes and can also be referred to as a mesh, net, or web. In FIG. 8, the top ends 62 and bottom ends 63 of the layers 56 of screen 16 include a top seal 64 and bottom seal 65 which provides for a mating surface, seat, and/or seal between the filter 4e and an inside wall surface 39i of a filter device 1b such as filter device 1c shown in FIG. 6. In an alternative embodiment, the screens 16 used in filter 4e can further include or be connected to an additional filter media 21 for mechanically trapping and/or adsorbing undesired matter 9 from a liquid, blood portion and blood 10. As also shown in FIG. 15, the filter 4e includes a splitter 82 which can cause a fluid, blood portion and blood 10 to be scattered and dispersed within the chamber 2 of a filter device 1 and structure of the filter 4e. The alternative filter 4e can be an integral component and be permanently sealed and contained within the chamber 2 of a filter device such as filter device 1c shown in FIG. 6, or can alternatively be a removable and replaceable cartridge 55 and be replaced when a filter device it also includes a screw-off or otherwise removable top portion 18 and seal 19, as shown in FIG. 22.

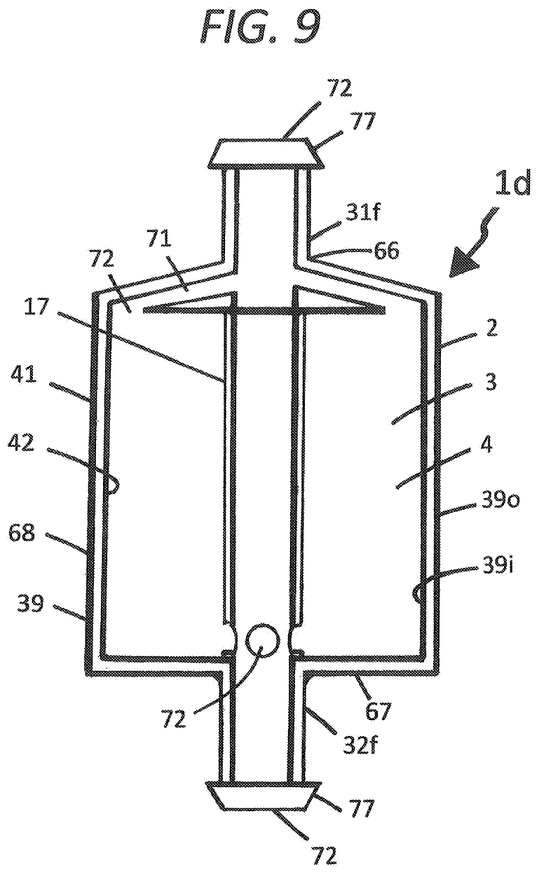

FIG. 9 is a side view of a filter device 1d which includes a wall 39 which can be made of a biocompatible material which can be a medical grade metal, glass, or thermoplastic material, e.g., polyamides or nylon, polyethylene glycol, polyvinyl alcohol, polyethyleneimine, polyvinylamine, polyacrylates, polyesters, polycarbonates, polystyrenes, polypropylene, polydimethylsiloxane or silicone, ABS, urethanes, nylon, PTFE, and a thermoplastic elastomer such as Santoprene. The wall 39 includes an outside wall surface 39o which is on the exterior 41 of the filter device 1d, and an inside wall surface 39i which is on the interior 42 of the filter device 1d. The filter device 1d can include an integral or removable inlet 31f and outlet 32f. The inlet 31f and outlet 32f can include barbs, threads, luer 94, or other types of male and female or hermathrodite structures for connecting and coupling the filter device 1d to tubes 7, and/or other devices or structures. The filter device 1d can have a top side 66, a bottom side 67, and one or more lateral side(s) 68 depending on whether the side 68 portion is configured as being circular in shape and has a radius oriented about a central axis 37, or other geometric shape. As shown in FIG. 9, the filter device 1d is configured with the inlet 31f and outlet 32f being in alignment which can enhance the flow through of a liquid, blood portion and blood 10. The structure of the filter device 1d defines a chamber 2 which includes a cavity 3 which includes a void space when empty and which has a volume. The filter device 1d can include a filter 4, such as filters 4c-e which are shown in FIGS. 5, 7 and 8. In an alternative embodiment, the screens 16 used in filter 4 can further include or be connected to an additional filter media 21 for mechanically trapping and/or adsorbing undesired matter 9 from a liquid, blood portion and blood 10. The filter 4c-e can be permanently sealed and contained within the interior chamber 2 of the filter device 1d shown in FIG. 9, or alternatively can be configured as a removable cartridge 55 and be removed when a filter device it also includes a screw-off or otherwise removable top portion 18 and seal 19, as shown in FIG. 22.

The filter device 1d can include a filter 4c-e which is used to help separate and remove undesired matter 9 such as metal, graphene oxide, graphene hydroxide, magnetic graphene oxide, pathogens, and toxins from an individual and patient's 28 blood 10. When a non-electrically charged filter 4 is being used, it can be helpful to use a permanent magnet 12, a plurality of magnetic beads 13, or an electromagnet 14 which produces an electromagnetic field and gradient 45 that imparts a positive charge proximate to the filter 4c-e. However, a filter 4c-e which includes an internal structure and/or media that is or can be electrically charged is typically selected for use. In this regard, the filter structure and/or media is typically positively electrically charged. As shown in FIGS. 5, 7, and 8, the filters 4c-e can include a filter layer 56 including a screen 16 which is made of an electrically conductive metal, ceramic, or other composite material. As shown in FIGS. 5, 7, and 8, a plurality of filter layers 56 can be disposed next to one another, and the filter layers 56 can be orientated parallel to the central axis 57 associated with the configuration of the inlet 31f and outlet 32f and direction of flow of a liquid, blood portion and blood 10 to form a complex matrix or maze 69. As shown in FIGS. 2 and 6, it can be readily understood that one or more magnet devices 11a-b can also be removably or permanently secured proximate to the wall 39, and in particular, the outside wall surface 39o of the filter device 1d. In this regard, the magnet devices 11 can be permanent magnets 12, a plurality of magnetic beads 13, or electromagnets 14. As shown in FIGS. 2 and 6, when a plurality of magnet devices 11a-b are used, the magnet devices 11a-b are typically orientated so that their north and south poles are configured to face the oppositive poles of the magnetic device 11a-b which is disposed nearby, that is, the south pole S of magnet device 11a is disposed opposite to the north pole N of magnet device 11b. In other words, a first magnet device 11a having a first positive pole and first negative pole is typically configured such that the first positive pole is disposed proximate to the second negative pole of the second magnet device 11b, and/or the first negative pole is disposed proximate to the second positive pole. In FIG. 2, the magnet devices 11a-b are shown disposed proximate to the top side 66 and bottom side 67 of the filter device 1a, whereas in FIG. 6 the magnet devices 11a-b are shown disposed proximate to the lateral side(s) 68 of the filter device 1c. As shown in FIG. 9, the path of a liquid, blood portion and blood 10 which enters the inlet 31f is directed by channels 71 towards the lateral side(s) 68 on the interior 42 of the filter device 1d, and the liquid, blood portion and blood 10 can then pass through the screen 16 and/or other media 21 portion of the filter 4c-e and into the perforated collection tube 17 which is connected to and permits a liquid, blood portion and blood 10 to flow out of the outlet 32f.

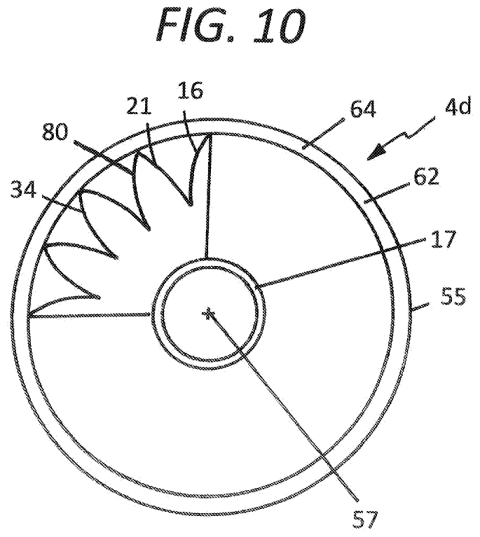

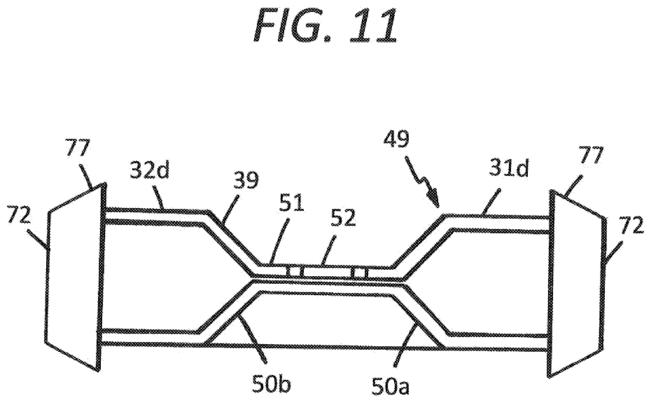

FIG. 10 is a top view of the filter 4d shown in FIG. 7. In this regard, the filter 4d is configured to have a convoluted structure 80 and can include a screen 16 made of wire 34 and/or other filter media 21 which surrounds a central perforated collection tube 17, and the filter 4d can be contained in the chamber 2 of a filter device 1, such as filter device 1d shown in FIG. 9. The filter 4d can alternatively further include a splitter 82 as shown in FIGS. 8 and 15. Alternatively, the filter 4d be configured as a removable and replaceable cartridge 55 and be removed when a filter device it is used which includes a screw-off or otherwise removable top portion 18 and seal 19, as shown in FIG. 22. FIG. 11 is a cross-sectional view taken along a line 11-11 of the blood dispersion device 49 shown in FIG. 16. As shown, the blood dispersion device 49 includes a inlet 31d, a funnel portion 50a, a middle portion 51 which is narrow in cross-section and can include a window 52 for optical monitoring of the contents of the blood dispersion device 49 such as a liquid, blood portion and blood 10, a funnel portion 50b, and an outlet 32d. At least one valve 30 or roller clamp 85 for starting, stopping, or regulating the flow of a liquid, blood portion and blood 10 can be disposed before inlet 32d and also after outlet 32d, or alternatively, a valve 30 can be included in the blood dispersion device 49. Accordingly, a liquid, blood portion and blood 10 contained in the blood dispersion device 49 can be made to stop and then be relatively motionless in order to enable optimal monitoring. The blood dispersion device 49 can have a thin cross-section in the middle portion 51 which is configured to provide a gap for blood flow that is less than or equal to 50 microns wide, or alternatively less than or equal to 30 microns wide, or alternatively in the range between 6-15 microns wide, or alternatively in the range between 1.5-15 microns wide. In this regard, a gap in the range between 6-15 microns wide can permit the relatively unobstructed passage and also the observation of platelets, red blood cells, and most white blood cells in an individual and patient's 28 blood 10. Once again, with regards to the size and scale used to discuss blood and its various components one micron (μm) equals 1000 nanometers (nm) and the average size of red blood cells is normally in the range between 6-9 microns or 6,000-9,000 nm. The average size of small white blood cells is in the range between 7-8 microns or 7,000-8,000 nm, whereas the average size of most white blood cells is in the range between 10-15 microns or 10,000-15,000 microns, but a few white blood cells are as large as 15-30 microns or 20,000-30,000 nm. The average size of platelets is typically between 1.5-4 microns or 1,500-4,000 nm. In order to not undesirably slow the flow of an individual and patient's blood 10 through an extracorporeal filtering device 33, a blood dispersion device 49 can possibly be connected and reconnected using two Y or T shaped connectors 84 and tubing 7 in parallel to the main flow path of a liquid, blood portion and blood 10 in an extracorporeal blood filtering device 33.

FIG. 12 is a top cross-sectional view taken along line 12-12 of the filter device 1e shown in FIG. 15. In this regard, the filter device 1e includes a first wall 39a having an outside wall surface 39o on the exterior 41 of the filter device 1e, and an inside wall surface 39i on the interior 42 of the filter device 1e. The filter device 1e, further includes a second wall 39b having outside portion 43, and an inside portion 44. A filter 4 such as filter 4c-e can be disposed within the central space defined by the inside portion 44 of the second wall 39b of the filter device 1e, whereas one or more magnet devices 11a-d can be disposed in the space defined between the outside portion 43 of the second wall 39b, and the inside wall surface 39i of the first wall 39a. Accordingly, the one or more magnet devices 11a-d can be placed in close proximity to a filter 4, such as a filter 4c-e. The magnet devices 11a-d which can be one or more permanent magnets 12, magnetic beads 13, or one or more electromagnets 14 can be selected for use, and also removed and replaced for possible future use. Further, the type and strength of the permanent magnet 12, magnetic beads 13, or electromagnet 14 can be selected to optimize the function and process of removing undesired matter 9 from a liquid, blood portion and blood 10 of an individual and patient 28, such as from a separated portion of blood plasma.

The magnet devices 11a-e are typically configured to produce an electromagnetic field and gradient 45 in range between 0.01-1 Tesla. For reference purposes, 1 Tesla is equal to 10,000 Gauss, and also 1 Volt (V) per second/(meters) m². Moreover, the Covid-19 virus is also negatively charged, and it is vulnerable to moderate electric fields. Electric field strengths between 10⁶ and 10⁷ Volts (V) Meter (m)⁻¹ which are below the breakdown threshold of water are commonly used in industrial food processing to inactivate pathogens, and electric field strengths less than or equal to 10⁴ Vm⁻¹ can possibly be used without adverse effects as discussed in the article: “The SARS-CoV-2 Spike Protein is Vulnerable To Moderate Electric Fields,” Sep. 13, 2021: https://www.nature.com/articles/s41467-021-25478-7. The isoelectric point of most viruses are found in the Ph range between 1.9-8.4, and in particular in the Ph range between 3.5-7, as discussed in the article: “Isoelectric Points of Viruses,” August, 2010: https://pubmcd.ncbi.nlm.nij.gov/20102425/. Most bacteria pathogens have a negative electrical charge, and magnetic beads 13 or nanoparticles which have a positive electrical charge can be used to capture, separate and remove them, as discussed in the articles: “Using Positively Charged Nanoparticles to Capture Bacteria at Ultralow Concentration,” Jun. 9, 2019: htups://nanoscalereslett.springeropen.corn/articles/10.1186/s11671-019-3005-z; and, “Electric Charge of Bacterial Antigens,” Apr. 1, 1932: https://www.jimmnunol.org/content/22/4/251.

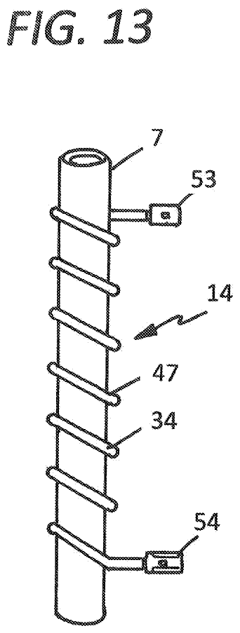

FIG. 13 is a perspective view of an electromagnet 14 which includes a coil 47 made of electrically conductive wire 34 or other electrically conductive matter which includes a male lead 53 and also a female lead 54 for connecting to positive and negative electrical wires and a source of electric power. The source of electric power can be DC or AC and come from a conventional power grid, a battery, solar panels or other form of renewable energy, a power supply, a computer 36 or a medical device which is connected by a power supply cord 97 to a wall plug 93. The electromagnet 14 can be disposed and encircle a tube 7 for carrying a liquid, blood portion and blood 10. When supplied with electric current it can impart an electromagnetic field and gradient 45 to undesired matter 9, and/or other substances contained in a liquid, blood portion and blood 10 and thereby elevate or otherwise modify the electric charge and/or manipulate undesired matter 9 and other substances contained in a liquid, blood portion and blood 10.

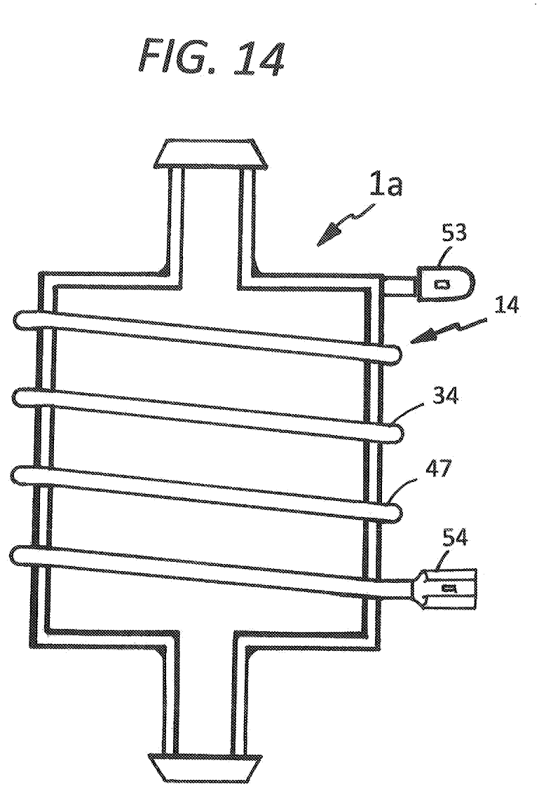

FIG. 14 is a side perspective view of an electromagnet 14 which includes a coil 47 made of electrically conductive wire 34 or other electrically conductive matter which includes a male lead 53 and also a female lead 54 for connecting to positive and negative electrical wires and a source of electric power. The source of electric power can be DC or AC and come from a conventional power grid, a battery, solar panels or other form of renewable energy, a power supply, a computer or a medical device which is connected to a wall plug 93. The electromagnet 14 can be disposed so at to coil around and encircle a filter device 1, such as filter device 1a for filtering a liquid, blood portion and blood 10. When the electromagnet 14 is supplied with electric current it can impart an electromagnetic field and gradient 45 to a filter 4 such as filter 4a-e and also to undesired matter 9, and other substances contained in a liquid, blood portion and blood 10, and thereby elevate or otherwise modify the electric charge of undesired matter 9, and/or manipulate other substances contained in a liquid, blood portion and blood 10. In this regard, the electromagnet 14 can impart an electromagnetic field and gradient 45 and create or enhance the positive charge of a filter 4, and also serve to attract, entrain, mix, move, or otherwise cause undesired matter 9 in the liquid, blood portion and blood 10 which typically have a negative charge to become captured, mechanically trapped, adsorbed, or otherwise attached to a filter 4 contained within a filter device 1 such as filter device 1a.

FIG. 15 is a side view of a filter device 1e which includes magnet devices 11a-e. The magnetic devices 11a-e can be made of permanent magnets 12, a plurality of magnetic beads 13, or electromagnets 14. As shown in FIG. 6, the electromagnets can include a coil 47 made of electrically conductive wire 34 or other electrically conductive matter disposed around a conductive core 46 and which include a male lead 53 and also a female lead 54 for connecting to positive and negative electrical wires or other sources of electric power. The source of electric power can be DC or AC and come from a conventional power grid, a battery, solar panels or other form of renewable energy, a computer or medical device which is connected with a power supply cord 97 to an electric outlet 93. The filter device 1e shown FIG. 15 includes a filter 4e similar to the one shown in FIG. 8 which includes a splitter 82 that can cause a stream of fluid, blood portion, or blood 10 to be scattered widely and dispersed within the structure of the filter 4e contained in the chamber 2 of the filter device 1e. The magnetic devices 11a-d can impart a plurality of electromagnetic fields and gradients 45 to a filter 4e and also to undesired matter 9 and other substances contained in liquid, a blood portion and blood 10, and thereby elevate or otherwise manipulate the electric charge and movement of undesired matter 9, and other substances contained in a liquid, blood portion and blood 10. When the magnetic devices 11a-d are configured as a plurality of electromagnets 14a-d they can be provided with electrical power simultaneously and constantly, or sequentially, or variably and in different patterns, and the polarity of the electromagnets 14a-d can also possibly be alternated and changed. In this regard, the electromagnets 14a-d can impart an electromagnetic field and gradient 45 and then create or enhance the positive charge of a filter 4 such as filter 4e, and also serve to attract, entrain, mix, move, or otherwise cause undesired matter 9 in a liquid, blood portion and blood 10 which typically has a negative charge to become captured, mechanically trapped, adsorbed, or otherwise attached to a filter 4 such as filter 4e contained within a filter device 1e.

FIG. 16 is a top view of a blood dispersion device 49 which can include an integral or removable inlet 31b and also outlet 32b. The inlet 31b and outlet 32b can include barbs, threads, luer 94, or other types of male and female or hermathrodite structures for connecting and coupling the blood dispersion device 49 to tubes 7, and/or other devices or structures. As shown in FIG. 16, the blood dispersion device 49 is configured with the inlet 31b and outlet 32b being in alignment about a central axis 57 which can enhance the flow through of a liquid, blood portion and blood 10. The blood dispersion device 49 includes a plurality of walls 39 which can be made of a biocompatible material which can be a medical grade metal, glass, or thermoplastic material, e.g., polyamides or nylon, polyethylene glycol, polyvinyl alcohol, polyethyleneimine, polyvinylamine, polyacrylates, polyesters, polycarbonates, polystyrenes, polypropylene, polydimethylsiloxane or silicone, ABS, urethanes, nylon, PTFE, and a thermoplastic elastomer such as Santoprene. The walls 39 include an outside wall surface 39o which is on the exterior 41 of the blood dispersion device 49, and an inside wall surface 39i which is on the interior 42 of the blood dispersion device 49. The structure of the blood dispersion device 49 defines a chamber 2 which includes a cavity 3 having void space when empty and which has a volume. The blood dispersion device 49 includes an inlet 31b which leads to a funnel portion 50a, a middle portion 51, and a funnel portion 50b which leads to an outlet 32b. The middle portion 51 can further include a window 52 for optical monitoring of the contents of the blood dispersion device 49. At least one valve 30 or roller clamp 85 for starting, stopping, or regulating the flow of a liquid, blood portion and blood 10 can be disposed before inlet 32d and also after outlet 32d, or alternatively, a valve 30 can be included in the blood dispersion device 49. The blood dispersion device 49 has a thin cross-section which is configured to provide a gap for blood flow that is less than or equal to 50 microns wide, or alternatively less than or equal to 30 microns wide, or alternatively in the range between 6-15 microns wide, or alternatively in the range between 1.5-10 microns wide. In this regard, a gap between 6-15 microns wide can permit the relatively unobstructed passage, and also the observation of red blood cells, white blood cells, and platelets in an individual and patient's 28 blood 10. Once again, with regards to the size and scale used to discuss blood and its various components one micron (μm) equals 1000 nanometers (nm) and the average size of red blood cells is normally in the range between 6-9 microns or 6,000-9,000 nm. The average size of small white blood cells is in the range between 7-8 microns or 7,000-8,000 nm, whereas the average size of most white blood cells is in the range between 10-15 microns or 10,000-15,000 microns, but a few white blood cells are as large as 15-30 microns or 20,000-30,000 nm. The average size of platelets is typically between 1.5-4 microns or 1,500-4,000 nm.

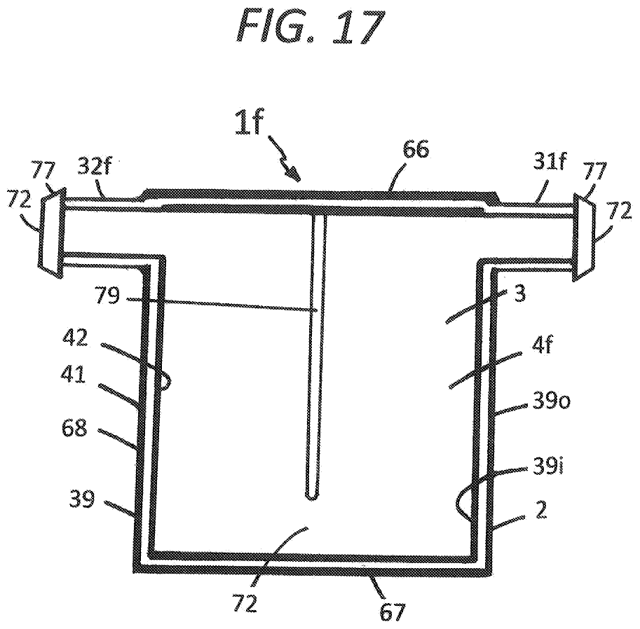

FIG. 17 is a side view of an alternative configuration of a T-shaped filter device if. The remaining structure and function of filter device if can be generally similar to that of filter devices 1a-e, which are shown in the other drawing figures and also discussed herein. However, the embodiment of a filter device if shown in FIG. 17 further includes a divider wall 79 which includes an opening 72 proximate to the bottom side 67 of the filter device if and this causes the flow of liquid, a blood portion or blood 10 to be directed downwards from the inlet 31f and upwards towards the outlet 32f.

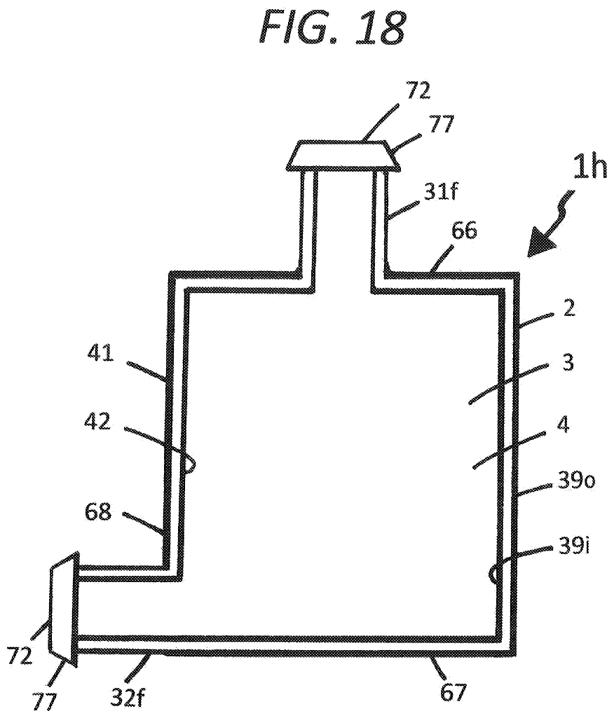

FIG. 18 is a side view of an alternative configuration of a L-shaped filter device 1h. The remaining structure and function of filter device 1h can be generally similar to that of filter devices 1a-e, which are shown in the other drawing figures and also discussed herein.

FIG. 19 is a side view of three components of a kit 91 for making a blood infusion or transfusion. In this regard, the kit 91 can include at least one needle 8 which can also be identified as 8a which is connected with a thermoplastic union 86 to a rubber or latex fitting 87 which is connected to tubing 7. The flow of a liquid, blood portion and blood in the tubing 7 can be controlled by at least one roller clamp 85 and/or valve 30. The tubing 7 which is connected to the rubber or latex fitting 87, union 86 and needle 8 is connected to a drip chamber 89 which is made at least in part of a flexible and at least partially transparent thermoplastic material which includes a conventional blood filter 90 which typically has a particle filtration size between 170-260 microns. In order to perform a transfusion, the kit 91 would typically further include another roller clamp 85, additional tubing 7, and another rubber or latex fitting 87, union 86, and needle 8 which can also be identified as 8b.

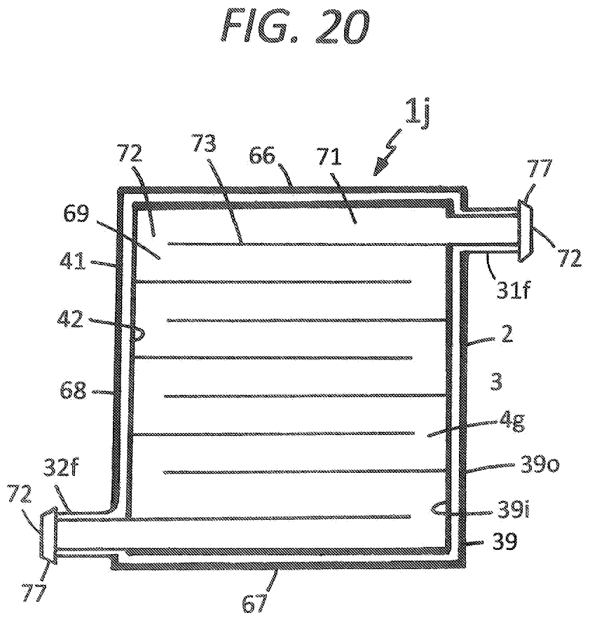

FIG. 20 is a side view of an alternative filter device 1j which includes a wall 39 which can be made of a biocompatible material which can be a medical grade metal, glass, or thermoplastic material, e.g., polyamides or nylon, polyethylene glycol, polyvinyl alcohol, polyethyleneimine, polyvinylamine, polyacrylates, polyesters, polycarbonates, polystyrenes, polypropylene, polydimethylsiloxane or silicone, ABS, urethanes, nylon, PTFE, and a thermoplastic elastomer such as Santoprene. The wall 39 includes an outside wall surface 39o which is on the exterior 41 of the filter device 1j, and an inside wall surface 39i which is on the interior 42 of the filter device 1j. The filter device 1j can include a top side 66, a bottom side 67, and one or more lateral side(s) 68 depending on whether the side 68 portion is configured to be circular in shape and has a radius oriented about a central axis 57, or other geometric shape. The filter device 1j can include an integral or removable inlet 31f and outlet 32f. The inlet 31f and outlet 32f can include barbs, threads, luer 94, or other types of male and female or hermathrodite structures for connecting and coupling the filter device 1j to tubes 7, and/or other devices or structures. As shown in FIG. 20, the filter device 1j is not configured with the inlet 31f and outlet 32f being in direct alignment, but rather the flow of a liquid, blood portion and blood 10 is directed through a plurality of internal channels 71 and openings 72 and plurality of internal baffles 73 resembling a matrix or maze 69 which serves to increase the exposure of a liquid, blood portion and blood 10 to the filter 4g which can include microstructures or nanostructures and/or filter media for trapping and/or adsorbing the undesired matter 9 to be separated and removed from a liquid, blood portion and blood 10. Alternatively, the inlet 31f and outlet 32f can be aligned about a central axis 57, or be otherwise disposed in functional relation to the filter device 4j. In FIG. 20, a total of eight internal baffles 73 and also eight openings 72 are shown, but the number of internal baffles 73, internal channels 71 and openings 72 can be in the range between 1 and a trillion. In this regard, the internal baffles 73 can be made of a thermoplastic or other materials which have been previously recited. Alternatively, the internal baffles 73 or other portions of the filter 4g can be made of a nanomaterial such as graphene, or graphene oxide, or magnetic graphene oxide, or other material which has a chemical, biological, mechanical, electromagnetic or other affinity to the undesired matter 9 which is targeted to be separated and removed from a liquid, blood portion and blood 10. As graphene oxide is known to have a natural tendency to congregate or aggregate and self-assemble, the use of the same family of material or an identical material as the target can be seen in some sense as “fighting fire with fire.” Alternatively, the filter 4g can include a nanomaterial which can be made of a biological material. In this regard, a biological material can possibly include, but not be limited to portions of DNA, RNA, or other biological molecules, cell structures, cells, natural or synthetic substances or a drug, e.g., heparin which is an anticoagulant, and can possibly include natural or synthetic liver or kidney tissue made outside of the human body in a laboratory and production facility. The structure of the filter device 1j defines a chamber 2 which includes a plurality of internal channels 71, baffles 73, and openings 72 and has a cavity 3 including a void space when empty and has a volume. In particular, the filter device 1j can include a filter 4g which is used to help separate and remove targeted undesired matter 9 such as metals, graphene oxide, graphene hydroxide, magnetic graphene oxide, pathogens, and toxins from an individual and patient's 28 blood portion or blood 10. When a non-electrically charged and/or a positively electrically charged filter 4g is being used, it can sometimes be helpful to dispose a magnetic device 11 which can be a permanent magnet 12, a plurality of magnetic beads 13, or an electromagnet 14 which produces an electromagnet field and gradient 45 that can impart an elevated electrical charge, and typically a positive charge proximate to the filter 4g. A filter 4g that already includes an internal structure and/or media which is and/or can be electrically charged is typically selected for use. In this regard, the filter 4g structure and/or media is typically positively electrically charged. The filter 4g can be permanently sealed and contained within the chamber 2 of the filter device 1j, or alternatively can be configured as a removable cartridge 55 and be inserted when a filter device it would include a screw-off or otherwise removable top portion 18 and seal 19, as shown in FIG. 22.

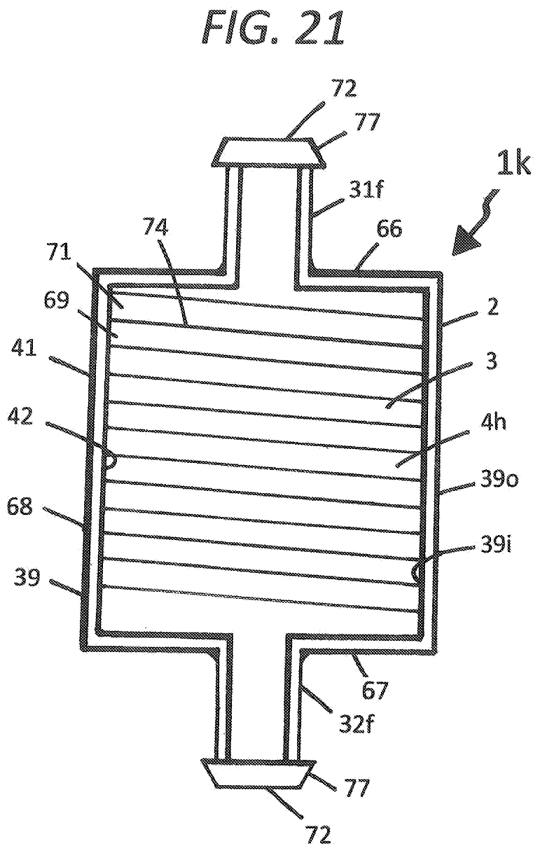

FIG. 21 is a side view of an alternative filter device 1k which includes a wall 39 which can be made of a biocompatible material which can be a medical grade metal, glass, or thermoplastic material, e.g., polyamides or nylon, polyethylene glycol, polyvinyl alcohol, polyethyleneimine, polyvinylamine, polyacrylates, polyesters, polycarbonates, polystyrenes, polypropylene, polydimethylsiloxane or silicone, ABS, urethanes, nylon, PTFE, and a thermoplastic elastomer such as Santoprene. The wall 39 includes an outside wall surface 39o which is on the exterior 41 of the filter device 1k, and an inside wall surface 39i which is on the interior 42 of the filter device 1k. The filter device 1k can have a top side 66, a bottom side 67, and one or more lateral side(s) 68 depending on whether the side 68 portion is configured to be circular in shape and has a radius oriented about a central axis 57, or other geometric shape. The filter device 1k can include an integral or removable inlet 31f and outlet 32f. The inlet 31f and outlet 32f can include barbs, threads, luer 94, or other types of male and female or hermathrodite structures for connecting and coupling the filter device 1k to tubes 7, and/or other devices or structures. As shown in FIG. 21, the filter device 1k can be configured with the inlet 31f and outlet 32f being in direct alignment, but the flow of a liquid, blood portion and blood 10 is directed through at least one internal channel 71 which has a spiral configuration 74 which serves to increase the exposure of a liquid, blood portion and blood 10 to the filter 4h which can include microstructures and/or filter media for trapping and/or adsorbing the targeted undesired matter 9 to be separated and removed from a liquid, blood portion and blood 10. Alternatively, the inlet 31f and outlet 32f can be offset as shown in FIG. 20, or be otherwise disposed in functional relation to the filter device 1k. In FIG. 21, a total of twelve descending internal channels 71 appear in the cross-sectional view, but they are part of a single internal channel 71 having a spiral configuration 74, or semi-spiral configuration which descends continuously, or alternatively, the channel 71 and spiral 74 structure can descend discontinuously and then include relatively flat, but also descending portions which have a slope in the range between 1/0 and 10/1. In this regard, the internal channel 71 can be made of thermoplastic and the other materials which have been previously recited, or alternatively of a nanomaterial such as graphene, or graphene oxide, or magnetic graphene oxide, or other material which has a chemical, biological, mechanical, electromagnetic or other affinity to the undesired matter 9 which is targeted to be separated and removed from a liquid, blood portion or blood 10. As graphene oxide is known to have a natural tendency to congregate or aggregate and self-assemble, the use of the same family of material or an identical material as the target can be seen in some sense as “fighting fire with fire.” Alternatively, the filter 4h can include a nanomaterial which can be made of a biological material. In this regard, a biological material can possibly include, but not be limited to portions of DNA, RNA, or other biological molecules, cell structures, cells, natural or synthetic substances or drugs, e.g., heparin which is an anticoagulant, and could include natural or synthetic liver or kidney tissue made outside of the human body in a laboratory and production facility. The structure of the filter device 1k defines a chamber 2 which is configured to include an internal channel 71 having a spiral configuration 74 and which includes a cavity 3 including a void space when empty and which has a volume. In particular, the filter device 1k can include a filter 4h which is used to help separate and remove undesired matter 9 such as metals, graphene oxide, graphene hydroxide, magnetic graphene oxide, pathogens, and toxins from an individual and patient's 28 blood portion or blood 10. When a non-electrically charged and/or a positively electrically charged filter 4h is being used, it can sometimes be helpful to dispose a magnetic device 11 which can be a permanent magnet 12, magnetic beads 13, or an electromagnet 14 which produces an electromagnetic field and gradient 45 that imparts an elevated electrical charge, and typically a positive charge proximate to the filter 4h in order to manipulate the undesired matter 9 and cause it to be separated and removed from a fluid, blood portion or blood 10. In this regard, a filter 4h that already includes an internal structure and/or media which is and/or can be electrically charged is typically selected for use. In this regard, the filter structure and/or media is typically positively electrically charged. The filter 4h can be permanently sealed and contained within the chamber 2 of the filter device 1k, or alternatively can be configured as a removable cartridge 55 and be replaced when the filter device it includes a screw-off or otherwise removable top portion 18 and seal 19, as shown in FIG. 22.

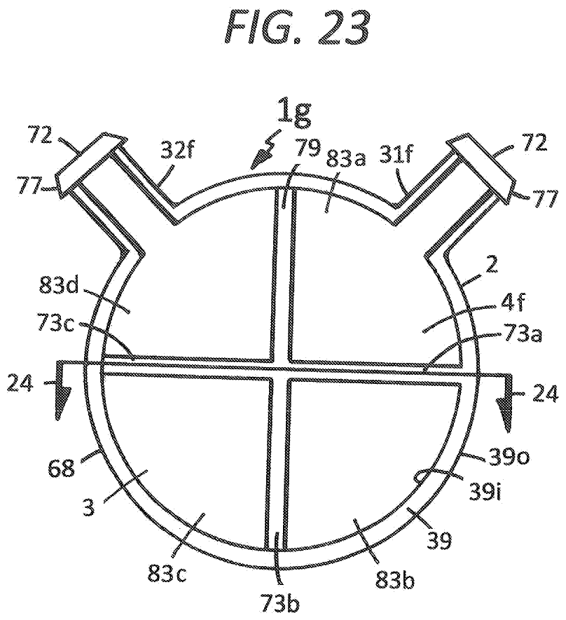

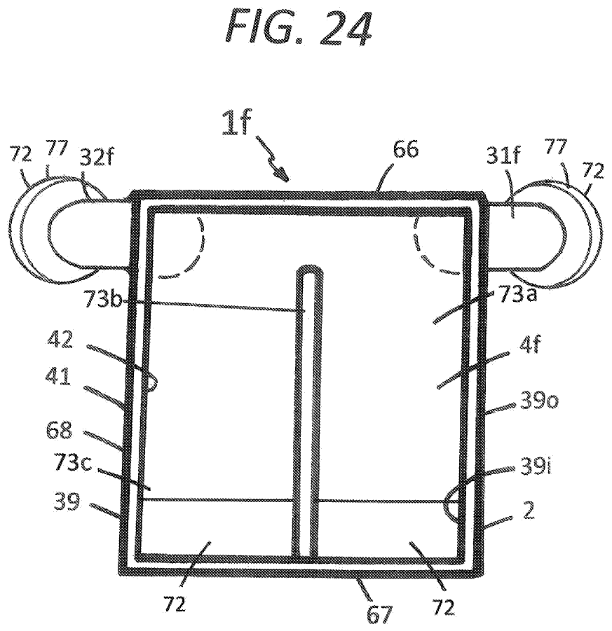

FIG. 23 is a top view of an alternative configuration of a T-shaped filter device 1g which includes a divider wall 79, and also three baffles 73a-c which are configured to define four quadrant 83a-d portions of the chamber 2 of the filter device 1g. In this regard, the first baffle 73a includes an opening 72 disposed proximate to the bottom side 67 of the filter device 1g and this causes a liquid, blood portion or blood 10 in the first quadrant 83a to flow downwards from the inlet 31f and into the bottom of the second quadrant 83b. The liquid, blood portion or blood 10 can then flow upwards in the second quadrant 83b and through an opening 72 disposed proximate to the top side 66 of the filter device 1g and into the third quadrant 83c. A liquid, blood portion or blood 10 can then flow downwards in the third quadrant 83c and through an opening 72 which is disposed proximate to the bottom side 67 of the filter device 1g and into the fourth quadrant 83d. The liquid, blood portion or blood 10 can then flow upwards in the fourth quadrant 83d and exit through the outlet 32f which includes an opening 72. FIG. 24 is a cross-sectional view of the filter device 1g shown in FIG. 23 taken along line 24-24 and shows baffles 73b-d which include openings 72 for directing the flow of a liquid, blood portion and blood 10 through the quadrants 83a-d disposed between the inlet 31f and outlet 32f.

### Magnet Device

Given the possible need or desire to remove metals or other electrically conductive and charged forms of undesired matter 9 from blood 10 which could include, but not be limited to members of the graphene family such as graphene oxide, graphene hydroxide, and magnetic graphene oxide, magnetic devices 11 which generate or impart electromagnetic fields and gradients 45 can be used to separate and remove undesired matter 9, pathogens, and toxic substances from the blood 10 of an individual and patient 28. A magnet device 11 can be either a permanent magnet device 12 or an electromagnet device 14. Permanent magnet devices 12 are made and generally available in the form of alnico magnets which typically include a composite of aluminum, nickel, and cobalt; ceramic magnets which typically include ferrite or iron oxide and barium or strontium carbonate; samarium cobalt (SmCo) magnets; neodymium iron boron (NeFeB) magnets; and, injection molded and/or flexible magnets. A permanent magnet device 12 can also be made and configured in the form of magnetic beads 13. Alternatively, a magnet device 11 can be an electromagnet device 14. In this regard, the electromagnet device 14 can possibly include a magnetic core 46 made of ferrite or other conductive material, and a coil 47 of wire 34 made of an electrically conductive metal such as steel, copper, silver, gold, aluminum, nickel, cobalt, or a conductive composite. However, a simple wire 34, and in particular, a coil 47 of wire 34 made of an electrically conductive metal will produce an electromagnetic field and gradient 45 when an electric current is caused to flow in such a wire 34 or coil 47. In accordance with the so-called “right hand rule,” the direction of the magnetic field will typically be perpendicular and clockwise relative to the direction of the movement or flow of the corresponding electric current.

As previously discussed, a magnet device 11 such as an electromagnet device 14 can be included in the second conditioning device 20, but also in the first conditioning device 5 and the third conditioning device 25. In this regard, the strength of an electromagnetic field and gradient 45 produced by the second conditioning device 20 and electromagnetic device 14 can be selectively increased or decreased as desired or required by manipulating a variable control 15a for voltage and current such as a variable resistor or potentiometer. Further, a variable control 15b for frequency and wavelength can be included and possibly be used to manipulate the frequency and wavelength of the electromagnetic field and gradient 45 being produced. In addition, the second conditioning device 20 can also include a variable control 15c for selecting the type of waveform being produced by the electromagnet device 14, e.g., a square wave, sinusoidal wave, sawtooth, or other waveform. Moreover, a variable control 15d for selecting whether a positive or negative magnetic pole and/or changing electromagnetic field and gradient 45 will be imparted to undesired matter 9 can also possibly be included with the second conditioning device 20 which includes an electromagnet device 14. Further, a variable control 15e for selecting between AC/DC can also be included in a second conditioning device 20 which includes an electromagnet device 14. A second conditioning device can also include a variable control 15f for selecting between power on/off. The magnetic device 11, which can be an electromagnetic device 14, the first conditioning device 5, second conditioning device 20 and third conditioning device and their variable controls 15a-f can be controlled by a computer 36 or medical device including a monitor screen 37, a keyboard 96, touch pad, and mouse. The magnetic device 11 which can be a permanent magnet 12 or electromagnet 14 is typically configured to produce an electromagnetic field and gradient 45 in range between 0.01-1 Tesla. Again, for reference purposes 1 Tesla is equal to 10,000 Gauss, and also 1 Volt (V) per second/(meters) m².

Moreover, the Covid-19 virus is also negatively charged, and it is vulnerable to moderate electric fields. Electric field strengths between 106 and 107 Volts (V) Meter (m)⁻¹ which are below the breakdown threshold of water are commonly used in industrial food processing to inactivate pathogens, and electric field strengths less than or equal to 10⁴ Vm⁻¹ can possibly be used without adverse effects as discussed in the article: “The SARS-CoV-2 Spike Protein is Vulnerable To Moderate Electric Fields,” Sep. 13, 2021: https://www.nature.com/articles/s41467-021-25478-7. The isoelectric point of most viruses are found in the Ph range between 1.9-8.4, and in particular in the Ph range between 3.5-7, as discussed in the article: “Isoelectric Points of Viruses,” August, 2010: https://pubmed.nebi.nim.nii.gov/20102425/. Most bacteria pathogens have a negative electrical charge, and magnetic beads 13 or nanoparticles which have a positive electrical charge can be used to capture, separate and remove them, as discussed in the articles “Using Positively Charged Nanoparticles to Capture Bacteria at Ultralow Concentration,” Jun. 9, 2019: https://nanoscalereslett.springeropen.con/articles/10.1186/s11671-019-3005-z, and “Electric Charge of Bacterial Antigens,” Apr. 1, 1932: https://www.jimmunol.org/content/22/4/251.

The following U.S. Patents disclose the use of magnets and electromagnetic fields having a gradient to separate and remove undesired matter from fluids and/or human blood, and some of these also make use of magnetic beads. U.S. Pat. No. 5,123,901 for “Method For Separating Pathogenic Or Toxic Agents From A Body Fluid And Return To Body” discloses using magnetic beads having a size in the range between 0.01-10 microns which also corresponds to a range between 100,000-1,000,000 paramagnetic beads per milliliter to remove the HIV-1 virus from human blood. U.S. Pat. No. 5,711,871 for “Magnetic Separation Apparatus” discloses such a device in FIG. 1, and also in the corresponding specification. U.S. Pat. No. 6,231,760 for “Apparatus For Mixing And Separation Employing Magnetic Particles,” discloses that configuring two magnets in an opposing relationship with their positive and negative poles each being close to one another causes there to be two areas having a high magnetic attraction, and a neutral center area. Further, this patent also discloses neodymium iron boron or samarium-cobalt magnetics characterized by a maximum energy product also known as BHmax of 25-45 MGOe, that is, megaGauss Oersted, which are made by International Magnaproducts, Inc. of Valparaiso, Indiana. In addition, this patent discloses sequential energization of electromagnets using simple binary on and off, but also analog energization with power being gradually increased and decreased, and then switching the polarity of magnets using 10-200 revolutions per minute to perform mixing. Moreover, this patent discloses using magnetic particles having a size range in diameter from 0.1 um to about 300 um, and a range in magnetic strength between about 200-5000 Gauss. U.S. Pat. No. 6,764,859 for “Device And Method For Mixing Magnetic Particles With A Fluid” discloses in Column 4, Lines 42-57 that having magnets face each other with similar poles helps to perform mixing. U.S. Pat. No. 7,699,979 for “Separation System And Efficient Capture Of Contaminants Using Magnetic Nanoparticles” discloses using magnetic nanoparticles and rotating magnetic fields to separate matter. This patent also suggests that a magnetic field having a strength of 1 Tesla which equals 10,000 Gauss is too high for use with human blood. U.S. Pat. No. 8,870,446 for “Device And Method For Manipulating And Mixing Magnetic Particles In A Liquid Medium,” and also U.S. Pat. No. 8,999,732 for “Method For Manipulating Magnetic Particles In A Liquid Medium” both disclose structures and methods of manipulating and mixing magnetic particles in a liquid. U.S. Pat. No. 9,150,631 for “Engineered Opsonin For Pathogen Detection And Treatment” discloses the use of magnetic beads made the following manufacturers: Dynal, Inc. which makes My One™ Dynabeads® located in Lake Success, New York; PerSeptive Diagnostics, Inc. located in Cambridge, Massachusetts; Invitrogen Corp. located in Carlsbad, California; Cortex Biochem, Inc. located in San Leandro, California; and, Bangs Laboratories located in Fishers, Indiana. U.S. Pat. No. 9,156,037 for “Microfluid Device And Uses Therefore” discloses using magnets to separate target components from fluid, and also the use of at least one collection channel, transfer channel, and removal channel. U.S. Pat. No. 9,389,225 for “Separating Target Analytes Using Alternating Magnetic Fields” discloses using alternating or shifting magnetic fields to separate magnetic and other matter from human blood. U.S. Pat. No. 10,265,457 for “Magnetic Filter Apparatus And Method,” discloses that the typical flow rate of blood to and from a human artery or vein for an adult is approximately between 40-400 milliliters (ml.) per minute (min.) with extracorporeal volume being about 8% of the total blood volume which in an adult having a body weight of 80 kilograms (kg) would be approximately 0.08×6.4 liters=512 ml. Further, this patent also discloses the use of neodymium iron boron magnets having 45 Newtons (N) of pull at 30 mm and 200 N of pull at 10 mm being disposed proximate to a filter including a plurality of wire screens, a blood pump, an air detector device, and also an air bubble removal device. This patent is assigned to MediSieve, Ltd. located in London, United Kingdom which makes the MediSieve single-use disposable magnetic filter which includes at least one removable and reusable permanent magnet, and can also be used with magnetic beads in order to filter a patient's total blood volume in less than an hour. For more information, see https://www.medisieve.com. U.S. Pat. No. 10,274,495 for “System And Method For Separating Cells Incorporating Magnetic Separation” discloses structures and methods for separating cells and matter from blood. U.S. Pat. No. 10,828,414, for “Magnetic Filtration Devices and Methods Relating Thereto” discloses in Column 22, at Lines 31-39: “Longer magnets magnetized across their length in the reversed orientation with like polarities repelling each other and minimal spacing between magnets exhibited strong efficacy in producing suitable magnetic fields for filtration of magnetic particle bound therapeutic agents.” U.S. 20100331753 A1 for “Blood Purification Method And Apparatus For The Treatment Of Malaria” discloses the use of magnetic fields to purify the blood of patients having malaria. All of the patents and patent applications recited in this paragraph are hereby incorporated by reference herein.

U.S. Patents relating to magnetic mixing devices include: U.S. Pat. Nos. 3,995,835, 6,764,859, 9,636,689, U.S. 20040114458 A1, U.S. 20050286342 A1, U.S. 20070207272 A1, and all of these patents are hereby incorporated herein. In addition, U.S. Patents relating to magnetic separation of particles or other matter from a solid or a liquid include: U.S. Pat. Nos. 3,567,026, 3,676,337, 3,902,994, 3,985,646, 4,054,513, 4,526,681, 4,663,029, 5,137,629, 5,465,849, 5,691,208, 6,361,749, 6,688,473, and all of these patents are hereby incorporated herein.

U.S. Patents relating to the use of magnetic particles or beads some of which include or disclose medical devices and methods for separating pathogens or toxins from human blood include: U.S. Pat. Nos. 3,531,413, 3,970,518, 3,933,997, 4,018,886, 4,177,253, 4,230,685, 4,267,234, 4,452,773, 4,554,088, 4,628,037, 4,659,678, 4,677,055, 4,695,393, 4,770,183, 4,795,698, 4,895,650, 4,910,148, 5,110,624, 5,123,901, 5,186,827, 5,200,084, 5,238,819, 5,240,855, 5,336,760, 5,385,707, 5,543,158, 5,578,325, 5,674,173, 5,695,946, 5,939,319, 6,033,574, 6,045,925, 6,676,729, 6,878,445, 7,462,446, 8,768,501, JP 58193687, WO 091/02811.

Further, U.S. Patents relating to binding members which can be used in separating pathogens or toxins from human blood include: U.S. Pat. Nos. 4,159,804, 4,219,411, 4,452,773, 4,710,472, 4,795,698, 5,200,084, and all of these patents are hereby incorporated herein.

In addition, U.S. Patents relating to devices and methods for use in identifying or separating pathogens or toxins from human blood include: U.S. Pat. Nos. 5,123,901, 5,439,586, 5,711,871, 5,951,877, 5,980,479, 6,051,146, 6,071,422, 6,153,113, 6,231,760, 6,280,622, 6,616,623, 7,758,533, 7,601,133, 7,699,979, 8,557,518, 8,841,104, 8,870,446, 8,999,732, 9,150,631, 9,156,037, 9,347,595, 9,389,225, 9,428,547, 9,593,160, U.S. U.S. Pat. Nos. 10,265,457, 10,274,495, 10,828,414, U.S. 2010021989 A1, U.S. 20100331753 A1, U.S. 20120065482 A1, U.S. 20130217144 A1, WO 2002094351 A3 WO 2009097151 A1, and all of these patents are hereby incorporated herein.

### Air Bubble Eliminator

The structures which are sometimes referred to in the medical field as being an air bubble eliminator, or an air bubble filter, or an air bubble trap will be referred to as being an air bubble eliminator 48 in the specification and air bubble eliminator in the claims. Examples of commercially available air bubble eliminators include the GVS SpeedFLow® 0.2 μm (micron) filter made by GVS Filtration, Inc. of Bologna, Italy which is available from TrueCare Biomedix USA Inc., of South Miami, Florida, and the B. Braun 0.2 μm SUPOR® filter made by B. Braun Medical, Inc. of Bethlehem, Pennsylvania and B. Braun Melsunger AG, located in Melsungen, Germany.

### Magnetic Beads

As briefly discussed above, magnetic beads 13 and/or magnetic nanoparticles of different types and compositions have been used in combination with permanent magnets 12 and/or electromagnets 14 in order to mix substances in fluids or blood, but also to separate and remove specific elements, pathogens or toxins in human blood. Magnetic beads 13 can be included inside a filter 4, and/or within the filter media of a filter 4. In this regard, the magnetic beads 13 can be electromagnetically attached to a conductive metal screen 16 of a filter 4. Further, magnetic beads 13 can be disposed proximate to a filter 4 and/or filter device 1. Moreover, magnetic beads 13 and/or magnetic nanoparticles can be placed in fluid communication with a fluid, blood portion and blood 10 of an individual and patient 28. Magnetic beads 13 can be used to help separate and remove metals, materials belonging to the graphene family, nanostructures, trackers, chips and other electronic devices, and/or to otherwise filter and treat the fluid, blood portion and blood of an individual and patient 28. In this regard, BD BioSciences located in San Jose, California makes flow cytometers, cell sorters, and also provides a Covid-19 ebook for identifying and separating different substances in human blood. The following article discloses that magnetic beads can be used in a process for testing for Covid-19, “A Magnetofluidic Platform For SARS-CoV-2 Variant And Respiratory Pathogen Detection:” https//doi.org/10.1101/2021.05.10.21256995, htpps://www.medrxiv.org/comment/10.1101/2021.05.10.21256995v1. Manufacturers of magnetic beads 13 for possible use include the following companies: AMSBIO located in Abingdon, United Kingdom which makes MagSi magnetic beads; IBA Lifesciences GmbH located in Göttingen, Germany which makes MagStrep Type3 XT beads and also a magnetic separator; Audemars Microtec located in Lamone-Cadempino, Switzerland which makes microbeads, microcoils, micromagnets and other related microcomponents; Bang Laboratories located in Fishers, Indiana which makes magnetic beads and microspheres; Cortex Biochem, Inc. located in San Leandro, California; Cube Biotech GmbH of Monheim am Rhein, Germany which makes nanodisks and other products relating to protein testing and purification; Dynal, Inc. which makes My One™ Dynabeads® located in Lake Success, New York; Invitrogen Corp. located in Carlsbad, California; MilliporeSigma located in Burlington, Massachusetts which also makes many other medical related products; Miltenyi Bioted GmbJ of Cologne, Germany which has a location in Cambridge, Massachusetts; PerSeptive Diagnostics, Inc. located in Cambridge, Massachusetts; and, Thermo Fisher Scientific located in Waltham, Massachusetts and having a sales office in Coon Rapids, Minnesota which makes Dynabeads™ including Streptavidin Dynabeads™ M-280, DynaMags™, and other medical related products.

### Pump Device

FIG. 30 shows a pump device 26 which has an inlet 31p, an outlet 32p. With reference to current medical practice in the United States, a pump device 26 will typically either be a roller type pump device or a centrifugal type pump device. The pump device 26 can include a variable blood flow control 29 whether onboard or remotely for regulating the speed at which an individual or patient's 28 blood 10 flows in the path or loop beginning from the first needle 8a puncture site 27a on their body 6 which is used for the extraction of their blood 10 and to the second needle 8b disposed at the second puncture site 27b on their body 6 where the treated blood 10 is returned to the individual or patient 28. The inlet 31 of the pump device 26 can be connected to or in fluid communication with the outlet 32 of the filter device 1 by tubing 7, and a valve 30 or roller clamp 85 can also be included for stopping or regulating the flow of blood 10 between the outlet 32 of the filter device 1 and inlet 31 of the pump device 26. Further, the outlet 32 of the pump device 26 can be connected by tubing 7 and also possibly to a valve 30 or roller clamp 85 for stopping or regulating the flow of blood 10 between the pump device 26 and the second needle 8b disposed at a second puncture site 27b for returning the individual or patient's treated blood 10 to their body 6 and bloodstream. For information relating to the history and development of blood pumps including early pulsatile tube compression pumps, but also the modern roller pump and centrifugal pump devices which are being used today, see, e.g., the article “History of Extracorporeal Circulation: The Invention And Modification Of Blood Pumps”: https://Pubmed.ncbi.nlm.nih.gov/14653417/. Once again, when medical equipment such as the pump device 26 is not available, as could be the case in some parts of the so-called third world, the use of a pump device 26 could possibly be omitted and the treatment procedure could simply use the individual's 28 normal blood pressure and also gravity, that is, provided that the first puncture site 27a used for extracting blood 10 would be disposed above the second puncture site 27b used for returning treated blood 10 to the body 6 of an individual and patient 28.

A pump device 26 can sometimes be already incorporated within and/or also be used in conjunction with other pieces of more sophisticated medical equipment for treating human blood. In this regard, depending upon the particular manufacturer and the model of medical equipment which is available for use and the particular need and requirement, one or more medical devices could be used for pumping blood and/or for also performing more complex tasks relating to extracorporeal blood purification (EBP), and such could include one or more of the following medical procedures: hemofiltration; continuous veno-venous hemofiltration; hemodiafiltration; ultrafiltration; hemodialysis/dialysis which typically uses dialysis filters which can remove molecules with molecular weights of 5,000-10,000 Dalton or more; and, plasmapheresis which typically uses plasma filters and/or centrifuge methods which can remove molecules with molecular weights of 1,000,000-5,000,000 Dalton or more.

The following companies are makers of blood pumps and/or equipment for treating and purifying blood. Baxter International located in Deerfield, Illinois makes the AMIA with SHARESOURCE remote patient monitoring platform, HomeChoice™, HomeChoice PRO™, and THERANOVA™ renal care products and Artificial Kidney AK 98 hemodialysis machine, and many infusion pumps such as model BM-11; B. Braun Medical, Inc. located in Bethlehem, Pennsylvania makes the Outlooko, Infusomato, Vistas, and Easypumpo infusion blood pumps, the Dialog+® Evolution Hemodialysis System, and Plasmat™ Futura System for H.E.L.P.® LDL Apheresis therapy; Medtronic PLC. located in Mounds View, Minnesota makes the Biomedicus Bio-Consoleo 500 series, the Affinity™ CP, and the BPX-80 Bio Pump™ blood pumps; Thoratec Corporation/Abbott located in Pleasanton, California makes the Thoratec, CentriMag®, and PediMag™, blood pumps; Terumo Corporation located in Somerset, New Jersey makes the Sams™, CAPIOX® SP, centrifugal blood pumps, Advanced Perfusion System 1 Roller Pump, and also CAPIOX®, Haemonetics®, LipiGuard®, Paul AutoVent SV™, and Pall LeukoGuard® filters, and CAPIOX® Bubble Traps; Qura Srl located in Mirandola, Italy makes centrifugal blood pumps, heat exchangers, and combinations thereof; Braile Biomedica, Ltd located in Sān Josè dio Rio Preto, Brazil makes the Centriflux® and Safira® centrifugal blood pumps and also blood filters; LivaNova, PLC. located in London, United Kingdom makes the Revolution® centrifugal blood pump and Intraoperative Autotransfusion System XTRA®; XENIOS AG/Fresenius Medical Care Company located in Heilbronn, Germany makes the Xenios® Diagonal pump, Xenios® Console for heart and lung support, and extracorporeal membrane oxygenation ECMO devices; Stockert Shiley had made roller blood pumps; and, Getinge AB located in Gothenburg, Sweden makes the Rotaflow RF-32 and RotaFlow II centrifugal blood pumps, Permanent Life Support (PLS) system, and Rotaflow Console. These and other products made by the aforementioned manufacturers can possibly be used in order to help remove metals and/or graphene oxide, graphene hydroxide, magnetic graphene oxide, or other undesired matter from the blood of individuals and patients.

Further, apheresis machines separate blood into its various components: red blood cells, white blood cells, platelets, and plasma. Examples of commercially available apheresis machines include: the thrombapheresis machine MAGELLAN® made by Arteriocyte Medical Systems located in Hopkinton, Massachusetts; the Bag-Extractor Bio15, Blood Component Extractor BIO45, and BagPRESS Bio45P made by Bioelecttronica located in Milan, Italy; the Benchtop Blood Component Extractor(s) TWIN6920 and ROBOTIC6960 made by BMS/Wuhan BMS Medicalteh Co., Ltd. located in Wuhan, China; the Plasmat Futura Apheresis System made by B. Braun located in Bethlehem, Pennsylvania; the Automatic Blood Component Extractor GIOTTO MONZA made by Delcon located in Grassobbio, Italy; the Automatic Blood Component Extractor DSEP series made by Demophorius Healthcare located in Limassol, Cyprus; the Erythropheresis Machine ALYS™ made by Fenwal™ located in Lake Zurich, Illinois; the Fractiomatic® Plus 2 Automated Blood Component Separator made by GRIFOLS located in Sant Cugat del Vallès, Spain; the plasmapheresis machine MCS+ 8150 made by ‘Haemonetics® located in Salt Lake City, Utah; the plasmapheresis machine XJ-II made by Haier Biomedical located in Sunbury Surrey, United Kingdom; the CF300 Plasma Adsorption Monitor made by INFOMED located in Geneva, Switzerland; the Autophereis-C Plasma Collection System, Aurora Plasma Collection System, and Alyx Apheresis Collection System made by Fresenius Kabi located in Lake Zurich, Illinois; the adsorption type plasma purification devices LIPOSORBER™ and Lixelle™ and the DX-21 apheresis machine made by Kaneka Medix Corporation located in Minato-Ku, Tokyo, Japan; the Automated Blood Component Extractor Luxomatic V2 made by LMB located in Schwaig, Germany; the leukapheresis machine LA25 and therapeutic apheresis machine AFTERSMART made by Medica located in Medolla, Italy; the ADAsorb® Therapeutic Apheresis System made by Medicap located in Ulrichstein, Germany; the transfusion apheresis machines TRIMA ACCEL® and Trim Accel® 7 made by Terumo located in Somerset, New Jersey; and, the plasmapheresis machine PCM made by Zhengyuan Technology Company, Ltd., (ZYT) located in China. Again, these and other products made by the aforementioned manufacturers can possibly be used in order to help remove metals and/or graphene oxide, graphene hydroxide, magnetic graphene oxide, or other undesired matter from the blood of individuals and patients.

In addition, when a medical practitioner is treating individuals and patients who are seriously ill and infected with the Covid-19 virus, other medical devices can possibly be used to remove the Covid-19 virus and/or cytokines from their bloodstream: e.g., the Extracorporeal Blood Purification (EBP) Device which can use the Oxiris® filter set and PrisMax® and Prismaflex® systems made by the Baxter Healthcare Corporation located in Deerfield, Illinois; the Seraph® 100 Microbind® Affinity Blood Filter and Extracorporeal Blood Purification (EBP) Device which use microbead adsorption media with chemically-bonded heparin which can serve to trap bacterial or virus pathogens which are made by ExThera Medical Corporation located in Martinez, California; and, the Extracorporeal Blood Purification (EBP) Device which is a cytokine adsorber which is made by CytoSorbents, Inc. located in Monmouth Junction, New Jersey; and, the Extracorporeal Blood Purification (EBP) Device which uses the Spectra Optia® Aphresis System with Marker Therapeutics' D2000 Adsorption cartridge to reduce the number of cytokines and other inflammatory mediators in an individual's bloodstream made by Terumo BCT, Inc. and Marker Therapeutics AG which are located in Lakewood, Colorado and Houston, Texas. In this regard, see the emergency use authorization of the FDA for the use of these devices in possibly correcting the blood 10 of individuals or patients 28 who are ill with the COVID-19 virus: https://www.fda.gov/medical-devices/coronavirus-disease-2019-covid-emergency-use-authorizations-medical-devices/blood-purification-devices-euas. Once again, these and other products made by the aforementioned manufacturers can possibly be used in order to help remove metals and/or graphene oxide, graphene hydroxide, magnetic graphene oxide, or other undesired matter from the blood of individuals and patients.

Some of the medical equipment, techniques, and procedures recited in the preceding paragraphs and including the U.S. Patents and U.S. Patent applications which have been incorporated by reference herein can possibly be used to separate and remove undesired matter 9 such as metals and members of the graphene family such as graphene oxide, graphene hydroxide, and magnetic graphene oxide from a liquid, blood portion and blood 10. Depending on the particular piece of medical equipment, and the expertise and techniques used by the operator and a medical professional, it is possible that some of the above recited equipment and procedures could be used. For example, an apheresis machine could be used to separate blood into its major components, that is, red blood cells, white blood cells, platelets, and plasma. Provided that the undesired matter 9 remains in the plasma and not in the other blood components, the plasma can then be filtered and the undesired matter 9 can be separated and removed. The plasma can then be recombined with the red blood cells, white blood cells, and platelets, and the blood be reinfused in an individual and patent 28. In this regard, it could be necessary or sound medical practice to then also use a blood warmer device. In addition, an air bubble detector could also be used to ensure that air bubbles have been successfully eliminated by what is sometimes referred to in the medical field as an air bubble eliminator 48, an air bubble filter, or an air bubble trap which is included and used with an extracorporeal blood filtering device 33 and/or another piece of medical equipment. Some of the recited pieces of medical equipment and the required medical expertise required to perform some of the aforementioned procedures may not be generally available to the public in the United States and also around the world. Further, over 200 million people have been “fully vaccinated” with one of the COVID-19 so-called vaccines, which is about 64 percent of the population in the United States, and so the potential pool of individuals and patients and the number of medical procedures to separate and remove undesired matter 9 from their bloodstream is very large. In addition, over 10 billion doses have been administered and over 60 percent of the world's population have received one of the so-called “vaccines.” Accordingly, the present disclosure recites several different alternative extracorporeal blood devices 33, methods, techniques, and processes for removing undesired matter from the blood of individuals and patients. The disclosed structures, devices, methods and techniques can be used to filter blood, or a blood portion such as plasma, or other fluid such as water.

The following Clauses provide exemplary filter devices for separating and removing undesired matter from blood or a blood component, and also exemplary methods of treating an individual having undesired matter dispersed in their blood.
Clause 1: A filter device for separating and removing undesired matter from blood or a component of blood comprising: a chamber comprising a top side, a bottom side, a lateral side, a central axis, a cylindrical shape, and a cavity; an outside height dimension between said top side and said bottom side of said chamber, said chamber comprising a maximum width dimension; an inlet disposed on said top side and an outlet disposed on said bottom side; a filter disposed in said cavity; said filter comprising at least one of a mesh, a screen, and a filter media capable of capturing, trapping, or adsorbing said undesired matter disposed on a plane orientated perpendicular to said central axis, whereby said blood or component of blood can be filtered in said chamber between said inlet and said outlet.
Clause 2: The filter device according to Clause 1, wherein said outside height dimension is equal to or greater than said maximum width dimension.
Clause 3: The filter device according to Clause 1, wherein said inlet and said outlet of are removable.
Clause 4: The filter device according to Clause 1, wherein said at least one of the mesh, the screen, and the filter media comprises a layer, and said filter comprises a plurality of layers.
Clause 5: The filter device according to Clause 1, wherein said at least one of the mesh, the screen, and the filter media comprises a border.
Clause 6: The filter device according to Clause 1, wherein said at least one of the mesh, the screen, and the filter media comprises a plurality of wires.
Clause 7: The filter device according to Clause 1, wherein said at least one of the mesh, the screen, and the filter media comprises a convoluted configuration.
Clause 8: The filter device according to Clause 4, wherein said plurality of layers comprises a nested configuration.
Clause 9: The filter device according to Clause 1, further comprising a magnet device disposed proximate to said filter.
Clause 10: The filter device according to Clause 9, said magnet device comprising at least one permanent magnet or electromagnet disposed proximate to at least one of said top side and said bottom side of said chamber, whereby said undesired matter can be captured, trapped, or adsorbed in said filter.
Clause 11: The filter device according to Clause 9, said magnet device comprising at least one permanent magnet or electromagnet disposed proximate to said lateral side of said chamber, whereby said undesired matter can be captured, trapped, or adsorbed in said filter.
Clause 12: The filter device according to Clause 9, said magnetic device comprising a first magnet having a first positive pole and a first negative pole, and a second magnet having a second positive pole and a second negative pole, said first magnet being configured relative to said second magnet such that the first positive pole of said first magnet is disposed opposite the second negative pole of said second magnet.
Clause 13: The filter device according to Clause 9, wherein said magnet device comprises an electromagnetic field in the range between 0.01-1.0 Tesla.
Clause 14: The filter device according to Clause 9, wherein said magnet device comprises an electromagnet comprising a coiled wire.
Clause 15: The filter device according to Clause 9, wherein said magnet device comprises an electromagnet comprising a wire coiled around a conductive core.
Clause 16: The filter device according to Clause 9, wherein said chamber comprises a support structure for removably securing said magnet device.
Clause 17: The filter device according to Clause 1, wherein said filter is configured to comprise an electrical charge.
Clause 18: The filter device according to Clause 1, wherein said filter comprises a plurality of magnetic beads.
Clause 19: The filter device according to Clause 1, wherein said filter comprises a screen or pore size smaller than about 1.5 microns, or 15 microns, or 30 microns.
Clause 20: The filter device according to Clause 1, wherein said undesired matter comprises at least one of a metal, graphene oxide, graphene hydroxide, magnetic graphene oxide, an electronic chip or other electronic device, a pathogen, and a toxin.
Clause 21: A filter device for separating and removing undesired matter from blood or a component of blood comprising: a chamber comprising a top side, a bottom side, a central axis, a curved lateral side, and a cylindrical shape; said chamber comprising a cavity; an outside height dimension between said top side and said bottom side of said chamber, said chamber comprising a maximum width dimension; an inlet disposed on said top side and an outlet disposed on said bottom side; said inlet configured to be aligned with said outlet and said central axis; a filter disposed in said cavity; said filter comprising at least one of a mesh, a screen, and a filter media capable of capturing, trapping, or adsorbing said undesired matter being disposed concentrically about and parallel to said central axis whereby said blood or component of blood can be filtered in said chamber between said inlet and said outlet.
Clause 22: The filter device according to Clause 21, wherein said outside height dimension is equal to or greater than said maximum width dimension.
Clause 23: The filter device according to Clause 21, wherein said inlet and said outlet are removable.
Clause 24: The filter device according to Clause 21, wherein said at least one of the mesh, the screen, and the filter media comprises a layer, and said filter comprises a plurality of layers.
Clause 25: The filter device according to Clause 21, wherein said at least one of the mesh, the screen, and the filter media comprises a border.
Clause 26: The filter device according to Clause 21, wherein said at least one of the mesh, the screen, and the filter media comprises a plurality of wires.
Clause 27: The filter device according to Clause 21, wherein said at least one of the mesh, the screen, and the filter media comprise a convoluted configuration.
Clause 28: The filter device according to Clause 24, wherein plurality of layers comprises a nested configuration.
Clause 29: The filter device according to Clause 21, further comprising a magnet device.
Clause 30: The filter device according to Clause 29, said magnet device comprising at least one permanent magnet or electromagnet disposed proximate to at least one of said top side and said bottom side of said chamber, whereby said undesired matter can be captured, trapped, or adsorbed in said filter.
Clause 31: The filter device according to Clause 29, said magnet device comprising at least one permanent magnet or electromagnet disposed proximate to said lateral side of said chamber, whereby said undesired matter can be captured, trapped, or adsorbed in said filter.
Clause 32: The filter device according to Clause 29, said magnetic device comprising a first magnet having a first positive pole and a first negative pole, and a second magnet having a second positive pole and a second negative pole, said first magnet being configured relative to said second magnet such that the positive pole of said first magnet is disposed opposite the negative pole of said second magnet.
Clause 33: The filter device according to Clause 29, wherein said magnet device comprises an electromagnetic field in the range between 0.01-1.0 Tesla.
Clause 34: The filter device according to Clause 29, wherein said magnet device comprises an electromagnet comprising a coiled wire.
Clause 35: The filter device according to Clause 29, wherein said magnet device comprises an electromagnet comprising a wire coiled around a conductive core.
Clause 36: The filter device according to Clause 29, wherein said chamber comprises a support structure for removably securing said magnet device.
Clause 37: The filter device according to Clause 21, wherein said filter is configured to comprise an electrical charge.
Clause 38: The filter device according to Clause 21, wherein said filter comprises a plurality of magnetic beads.
Clause 39: The filter device according to Clause 21, wherein said filter comprises a screen or pore size smaller than 1.5 half microns, or 15 microns, or 30 microns.
Clause 40: The filter device according to Clause 21, wherein said undesired matter comprises at least one of a metal, graphene oxide, graphene hydroxide, magnetic graphene oxide, an electronic chip or other electronic device, a pathogen, and a toxin.
Clause 41: A filter device for separating and removing undesired matter from blood or a component of blood comprising: a chamber comprising a top side, a bottom side, a central axis, a curved lateral side, and a cylindrical shape; said chamber comprising a cavity; an outside height dimension between said top side and said bottom side of said chamber, said chamber comprising a maximum width dimension; an inlet disposed on said top side and an outlet disposed on said bottom side; said inlet configured to be aligned with said outlet about said central axis; said inlet being in fluid communication with at least one channel which is in fluid communication with said cavity; said chamber comprising a perforated tube orientated parallel to and about said central axis; said perforated tube being in fluid communication with said outlet; a filter disposed in said cavity of said chamber; said filter comprising at least one of a mesh, a screen, and a filter media capable of capturing, trapping or adsorbing said undesired matter disposed between said top side and said bottom side; said at least one of the mesh, the screen, and the filter media being disposed concentrically about and parallel to said central axis; whereby said blood or component of blood can be filtered in said chamber between said inlet and said outlet.
Clause 42: The filter device according to Clause 41, wherein said outside height dimension is equal to or greater than said maximum width dimension.
Clause 43: The filter device according to Clause 41, wherein said inlet and said outlet are removable.
Clause 44: The filter device according to Clause 41, wherein said at least one of the mesh, the screen, and the filter media comprises a layer, and said filter comprises a plurality of layers.
Clause 45: The filter device according to Clause 41, wherein said at least one of the mesh, the screen, and the filter media comprises a border.
Clause 46: The filter device according to Clause 41, wherein said at least one of the mesh, the screen, and the filter media comprises a plurality of wires.
Clause 47: The filter device according to Clause 41, wherein said at least one of the mesh, the screen, and the filter media comprises a convoluted configuration.
Clause 48: The filter device according to Clause 44, wherein said plurality of layers comprise a nested configuration.
Clause 49: The filter device according to Clause 41, further comprising a magnet device.
Clause 50: The filter device according to Clause 49, said magnet device comprising at least one permanent magnet or electromagnet disposed proximate to at least one of said top side and said bottom side of said chamber, whereby said undesired matter can be captured, trapped, or adsorbed in said filter.
Clause 51: The filter device according to Clause 49, said magnet device comprising at least one permanent magnet or electromagnet disposed proximate to said lateral side of said chamber, whereby said undesired matter can be captured, trapped or adsorbed in said filter.
Clause 52: The filter device according to Clause 49, said magnetic device comprising a first magnet having a first positive pole and a first negative pole, and a second magnet having a second positive pole and a second negative pole, said first magnet being configured relative to said second magnet such that said first positive pole of said first magnet is disposed opposite said second negative pole of said second magnet.
Clause 53: The filter device according to Clause 49, wherein said magnet device comprises an electromagnetic field in the range between 0.01-1.0 Tesla.
Clause 54: The filter device according to Clause 49, wherein said magnet device comprises an electromagnet comprising a coiled wire.
Clause 55: The filter device according to Clause 49, wherein said magnet device comprises an electromagnet comprising a wire coiled around a conductive core.
Clause 56: The filter device according to Clause 49, wherein said chamber comprises a support structure for removably securing said magnet device.
Clause 57: The filter device according to Clause 41, wherein said filter is configured to comprise an electrical charge.
Clause 58: The filter device according to Clause 41, wherein said filter comprises a plurality of magnetic beads.
Clause 59: The filter device according to Clause 41, wherein said filter comprises a screen or pore size smaller than 1.5 microns, or 15 microns, or 30 microns.
Clause 60: The filter device according to Clause 41, wherein said undesired matter comprises at least one of a metal, graphene oxide, graphene hydroxide, magnetic graphene oxide, an electronic chip or other electronic device, a pathogen, and a toxin.
Clause 61: A filter device for separating and removing undesired matter from blood or a component of blood comprising: a chamber comprising a top side, a bottom side, a central axis, a curved lateral side, and a cylindrical shape; said chamber comprising a cavity; an outside height dimension between said top side and said bottom side of said chamber, said chamber comprising a maximum width dimension; an inlet disposed on said top side and an outlet disposed on said bottom side; said inlet configured to be aligned with said outlet and about said central axis; said chamber comprising a first wall comprising a first outside surface and a first inside surface and comprising a first radius; said chamber comprising a second wall comprising a second outside surface and a second inside surface and comprising a second radius; said second wall being concentric to said first wall and said second radius being less than said first radius; said second wall defining a first space in said cavity of said chamber between said first inside surface of said first wall and said second outside surface of said second wall; said second wall defining a second space in said cavity of said chamber between opposite portions of said second inside wall; a filter comprising at least one of a mesh, a screen, and a filter media capable of capturing, trapping or adsorbing said undesired matter disposed in said cavity of said chamber in said second space; and, a magnetic device disposed in said cavity of said chamber in said first space, whereby said magnetic device can impart an electromagnetic field upon said filter and said undesired matter contained in said blood or component of blood in said chamber and cause said undesired matter to be captured, trapped, or adsorbed in said filter.
Clause 62: The filter device according to Clause 61, wherein said outside height dimension is equal to or greater than said maximum width dimension.
Clause 63: The filter device according to Clause 61, wherein said inlet and said outlet are removable.
Clause 64: The filter device according to Clause 61, wherein said at least one the mesh, the screen, and the filter media comprises a layer, and said filter comprises a plurality of layers.
Clause 65: The filter device according to Clause 61, wherein said at least one of the mesh, the screen, and the filter media comprises a border.
Clause 66: The filter device according to Clause 61, wherein said at least one of the mesh, the screen, and the filter media comprises a plurality of wires.
Clause 67: The filter device according to Clause 61, wherein said at least one of the mesh, the screen, and the filter media comprises a convoluted configuration.
Clause 68: The filter device according to Clause 64, wherein said plurality of layers comprise a nested configuration.
Clause 69: The filter device according to Clause 61, said magnet device comprising at least one permanent magnet or electromagnet disposed proximate to at least one of said top side and said bottom side of said chamber, whereby said undesired matter can be captured, trapped, or adsorbed in said filter.
Clause 70: The filter device according to Clause 61, said magnet device comprising at least one permanent magnet or electromagnet disposed proximate to said lateral side of said chamber, whereby said undesired matter can be captured, trapped or adsorbed in said filter.
Clause 71: The filter device according to Clause 61, said magnetic device comprising a first magnet having a first positive pole and a first negative pole, and a second magnet having a second positive pole and a second negative pole, said first magnet being configured relative to said second magnet such that said first positive pole of said first magnet is disposed opposite said second negative pole of said second magnet.
Clause 72: The filter device according to Clause 61, wherein said magnet device comprises an electromagnetic field in the range between 0.01-1.0 Tesla.
Clause 73: The filter device according to Clause 61, wherein said magnet device comprises an electromagnet comprising a coiled wire.
Clause 74: The filter device according to Clause 61, wherein said magnet device comprises an electromagnet comprising a wire coiled around a conductive core.
Clause 75: The filter device according to Clause 61, wherein said filter is configured to comprise an electrical charge.
Clause 76: The filter device according to Clause 61, wherein said filter comprises a plurality of magnetic beads.
Clause 77: The filter device according to Clause 61, wherein said filter comprises a screen or pore size smaller than 1.5 microns, or 15 microns, or 30 microns.
Clause 78: The filter device according to Clause 61, wherein said undesired matter which is electrically conductive comprises at least one of a metal, graphene oxide, graphene hydroxide, magnetic graphene oxide, an electronic chip or other electronic device, a pathogen, and a toxin.
Clause 79: A filter device for separating and removing undesired matter from blood or a component of blood comprising: a chamber comprising a top side, a bottom side, a curved lateral side, and a cylindrical shape; said chamber comprising a cavity; an outside height dimension between said top side and said bottom side of said chamber, said chamber comprising a maximum width dimension; said chamber comprising an inlet and an outlet; said chamber comprising a spiral channel which descends from proximate said top side to proximate said bottom side; a filter disposed within said spiral channel, whereby the length of the flow path of said blood or blood component is greater than said outside height dimension.
Clause 80: The filter device according to Clause 79, wherein said outside height dimension is equal to or greater than said maximum width dimension.
Clause 81: The filter device according to Clause 79, wherein said inlet and said outlet are removable.
Clause 82: The filter device according to Clause 79, wherein said filter comprises at least one layer of a mesh, a screen, and a filter media capable of capturing, trapping, or adsorbing said undesired matter.
Clause 83: The filter device according to Clause 82, wherein said at least one layer of the mesh, the screen, and the filter media comprises a border.
Clause 84: The filter device according to Clause 82, wherein said at least one layer of the mesh, the screen, and the filter media comprises a plurality of wires.
Clause 85: The filter device according to Clause 82, wherein said at least one layer comprises a convoluted configuration.
Clause 86: The filter device according to Clause 82, wherein said at least one layer comprises a plurality of layers comprising a nested configuration.
Clause 87: The filter device according to Clause 79, further comprising a magnet device.
Clause 88: The filter device according to Clause 87, said magnet device comprising at least one permanent magnet or electromagnet disposed proximate to at least one of said top side and said bottom side of said chamber, whereby said undesired matter can be captured, trapped, or adsorbed in said filter.
Clause 89: The filter device according to Clause 87, said magnet device comprising at least one permanent magnet or electromagnet disposed proximate to said lateral side of said chamber, whereby said undesired matter can be captured, trapped, or adsorbed in said filter.
Clause 90: The filter device according to Clause 87, said magnetic device comprising a first magnet having a first positive pole and a first negative pole, and a second magnet having a second positive pole and a second negative pole, said first magnet being configured relative to said second magnet such that said first positive pole of said first magnet is disposed opposite said second negative pole of said second magnet.
Clause 91: The filter device according to Clause 87, wherein said magnet device comprises an electromagnetic field in the range between 0.01-1.0 Tesla.
Clause 92: The filter device according to Clause 87, wherein said magnet device comprises an electromagnet comprising a coiled wire.
Clause 93: The filter device according to Clause 87, wherein said magnet device comprises an electromagnet comprising a wire coiled around a conductive core.
Clause 94: The filter device according to Clause 79, wherein said chamber comprises a support structure for removably securing said magnet device.
Clause 95: The filter device according to Clause 79, wherein said filter is configured to comprise an electrical charge.
Clause 96: The filter device according to Clause 79, wherein said filter comprises a plurality of magnetic beads.
Clause 97: The filter device according to Clause 79, wherein said filter comprises a screen or pore size smaller than 1.5 microns, or 15 microns, or 30 microns.
Clause 98: The filter device according to Clause 79, wherein said undesired matter comprises at least one of a metal, graphene oxide, graphene hydroxide, magnetic graphene oxide, an electronic chip or other electronic device, a pathogen, and a toxin.
Clause 99: The device according to Clause 79, wherein said spiral channel descends from proximate said top side to proximate said bottom side in a plurality of portions which alternate in slope in the range between 0/1 and 10/1.
Clause 100: A filter device for separating and removing undesired matter from blood or a component of blood comprising: a chamber comprising a top side, a bottom side, and a lateral side; said chamber comprising a cavity; an outside height dimension between said top side and said bottom side of said chamber, said chamber comprising a maximum width dimension; an inlet disposed on said top side and an outlet disposed on said bottom side; said chamber comprising a plurality of baffles defining a plurality of channels and openings; each of said plurality of channels being in fluid communication with each other of said plurality of channels through said openings; one or more filters disposed within said plurality of channels; whereby the length of the flow path of said blood or blood component is greater than said outside height dimension.
Clause 101: The filter device according to Clause 100, wherein said outside height dimension is equal to or greater than said maximum width dimension.
Clause 102: The filter device according to Clause 100, wherein said inlet and said outlet are removable.
Clause 103: The filter device according to Clause 100, wherein said filter comprises at least one layer of a mesh, a screen, and a filter media capable of capturing, trapping, or adsorbing said undesired matter.
Clause 104: The filter device according to Clause 103, wherein said at least one layer of the mesh, the screen, and the filter media comprises a border.
Clause 105: The filter device according to Clause 103, wherein said at least one layer of the mesh, the screen, and the filter media comprises a plurality of wires.
Clause 106: The filter device according to Clause 103, wherein said at least one layer comprises a convoluted configuration.
Clause 107: The filter device according to Clause 103, wherein said at least one layer comprises a plurality of layers comprising a nested configuration.
Clause 108: The filter device according to Clause 100, further comprising a magnet device.
Clause 109: The filter device according to Clause 108, said magnet device comprising at least one permanent magnet or electromagnet disposed proximate to at least one of said top side and said bottom side of said chamber, whereby said undesired matter can be captured, trapped, or adsorbed in said filter.
Clause 110: The filter device according to Clause 108, said magnet device comprising at least one permanent magnet or electromagnet disposed proximate to said lateral side of said chamber, whereby said undesired matter which is electrically conductive can be captured, trapped, or adsorbed in said filter.
Clause 111: The filter device according to Clause 108, said magnetic device comprising a first magnet having a first positive pole and a first negative pole, and a second magnet having a second positive pole and a second negative pole, said first magnet being configured relative to said second magnet such that said first positive pole of said first magnet is disposed opposite said second negative pole of said second magnet.
Clause 112: The filter device according to Clause 108, wherein said magnet device comprises an electromagnetic field in the range between 0.01-1.0 Tesla.
Clause 113: The filter device according to Clause 108, wherein said magnet device comprises an electromagnet comprising a coiled wire.
Clause 114: The filter device according to Clause 108, wherein said magnet device comprises an electromagnet comprising a wire coiled around a conductive core.
Clause 115: The filter device according to Clause 108, wherein said chamber comprises a support structure for removably securing said magnet device.
Clause 116: The filter device according to Clause 100, wherein said filter is configured to comprise an electrical charge.
Clause 117: The filter device according to Clause 100, wherein said filter comprises a plurality of magnetic beads.
Clause 118: The filter device according to Clause 100, wherein said filter comprises a screen or pore size smaller than 1.5 microns, or 15 microns, or 30 microns.
Clause 119: The filter device according to Clause 100, wherein said undesired matter comprises at least one of a metal, graphene oxide, graphene hydroxide, magnetic graphene oxide, an electronic chip or other electronic device, a pathogen, and a toxin.
Clause 120: A method of treating an individual having undesired matter dispersed in their blood, the method comprising the steps of: inserting a first needle into a first vein or artery of the individual at a first puncture site on the individual and connecting the first needle to at least one piece of tubing, a stop valve, a filter device comprising at least one of a mesh, a screen, and a filter media which is capable of capturing, trapping, or adsorbing said undesired matter, an air bubble eliminator, and to a second needle; inserting the second needle into a second vein or artery of the individual at a second puncture site, the second puncture site being disposed at a lower vertical elevation than the first puncture site; and, manipulating the stop valve to initiate the flow of blood from the first puncture site through the filter device and reinfusing filtered blood through the second puncture site.
Clause 121: The method according to Clause 120, further comprising a magnet device comprising at least one permanent magnet or electromagnet disposed proximate to said filter.
Clause 122: The method according to Clause 121, said magnet device comprising a first magnet having a first positive pole and also a first negative pole, and a second magnet having a second positive pole and also a second negative pole, said first magnet being configured relative to said second magnet such that said first positive pole of said first magnet is disposed opposite said second negative pole of said second magnet.
Clause 123: The method according to Clause 121, wherein said magnet device comprises an electromagnetic field in the range between 0.01-1.0 Tesla.
Clause 124: The method according to Clause 121, wherein said magnet device comprises at least one selection control for controlling at least one of the following electrical or electromagnetic variables: the voltage and current, the frequency and wavelength, the waveform, the polarity, the source of electrical power being either AC versus DC, and an on and off switch.
Clause 125: The method according to Clause 120, said filter device being configured to comprise an electrical charge whereby said undesired matter can be captured, trapped, or adsorbed in said filter device.
Clause 126: The method according to Clause 120, said filter device further comprising a plurality of magnetic beads, whereby said undesired can be captured, trapped, or adsorbed in said filter device.
Clause 127: The method according to Clause 120, wherein said undesired matter which comprises at least one of a metal, graphene oxide, graphene hydroxide, magnetic graphene oxide, an electronic chip or other electronic device, a pathogen, and a toxin.
Clause 128: The method according to Clause 120, wherein said flow is at rate selected from at least one the following rates: 0.1-10 L/hr, 1-10 L/hr, 3-10 L/hr, or 5-10 L/hr.
Clause 129: The method according to Clause 120, wherein said flow and said reinfusion is performed in a continuous mode, or a cyclic mode.
Clause 130: The method according to Clause 120, wherein said filter comprises a screen or pore size smaller than 1.5 microns, or 15 microns, or 30 microns.
Clause 131: The method according to Clause 120, wherein said inlet and said outlet of said filter device are removable.
Clause 132: A method of treating an individual having undesired matter which is dispersed in their blood, the method comprising the steps of: inserting a first needle into a first vein or artery of the individual at a first puncture site on the individual and connecting the first needle to at least one piece of tubing, a stop valve, a filter device comprising at least one of a mesh, a screen, and a filter media which is capable of capturing, trapping, or adsorbing said undesired matter, an air bubble eliminator, a pump, and to a second needle; inserting the second needle into a second vein or artery of the individual at a second puncture site; and, manipulating the stop valve to initiate the flow of blood from the first puncture site through the filter device and reinfusing filtered blood through the second puncture site.
Clause 133: The method according to Clause 132, further comprising connecting at least one analyte sensor to said tubing between said first needle and said filter device.
Clause 134: The method according to Clause 132, further comprising connecting at least one analyte sensor to said tubing between said filter device and said second needle.
Clause 135: The method according to Clause 132, further comprising connecting at least one analyte sensor in said filter device.
Clause 136: The method according to Clause 132, further comprising connecting a blood or blood portion dispersion to said tubing between said first needle and said filter device.
Clause 137: The method according to Clause 132, further comprising connecting a blood or blood portion dispersion device to said tubing between said filter device and said second needle.
Clause 138: The method according to Clause 132, further comprising using at least one optical device to observe said blood between said first needle and said filter device.
Clause 139: The method according to Clause 132, further comprising using at least one optical device to observe said blood between said filter device and said second needle.
Clause 140: The method according to Clause 132, further comprising using at least one optical device to observe said blood in said filter device.
Clause 141: The method according to Clause 132, further comprising using a first blood or blood component conditioning device to condition said blood between said first needle and said filter device.
Clause 142: The method according to Clause 132, further comprising using a second blood or blood component conditioning device to condition said blood in said filter device.
Clause 143: The method according to Clause 142, said second blood or blood component conditioning device comprising a magnet device comprising at least one permanent magnet or electromagnet for providing an electromagnetic field.
Clause 144: The method according to Clause 143, said magnet device comprising a first magnet having a first positive pole and a first negative pole, and a second magnet having a second positive pole and a second negative pole, said first magnet being configured relative to said second magnet such that said first positive pole of said first magnet is disposed opposite said second negative pole of said second magnet.
Clause 145: The method according to Clause 143, wherein said electromagnetic field is in the range between 0.01-1.0 Tesla.
Clause 146: The method according to Clause 132, further comprising using a third blood or blood component conditioning device for conditioning said blood between said filter device and said second needle.
Clause 147: The method according to Clause 132, further comprising monitoring at least one of said stop valve, said filter, said pump, said air bubble eliminator and said flow of blood.
Clause 148: The method according to Clauses 133-147, further comprising monitoring at least one of an analyte sensor, a blood dispersion device, an optical device, a blood or blood component conditioning device, and said magnet device.
Clause 149: The method according to Clause 147, wherein said optical device is at least one of a camera, a microscope, a stereo microscope, and an electron microscope.
Clause 150: The method according to Clause 147, wherein said blood or blood component conditioning device comprises at least one selection control for controlling at least one of the following electrical or electromagnetic variables: the voltage and current, the frequency and wavelength, the waveform, the polarity of at least one electromagnet, the source of electrical power being either AC versus DC, and an on and off switch.
Clause 151: The method according to Clause 132, said filter device being configured to comprise an electrical charge whereby said undesired matter can be captured, trapped, or adsorbed in said filter device.
Clause 152: The method according to Clause 132, said filter device further comprising a plurality of magnetic beads.
Clause 153: The method according to Clause 132, where said undesired matter comprises at least one of a metal, graphene oxide, graphene hydroxide, magnetic graphene oxide, an electronic chip or other electronic device, a pathogen, and a toxin.
Clause 154: The method according to Clause 132, wherein said flow is at rate selected from at least one the following rates: 0.1-10 L/hr, 1-10 L/hr, 3-10 L/hr, or 5-10 L/hr.
Clause 155: The method according to Clause 132, wherein said flow and said reinfusion is performed in a continuous mode, or a cyclic mode.
Clause 156: The method according to Clause 132, wherein said filter comprises a screen or pore size smaller than 1.5 microns, or 15 microns, or 30 microns.
Clause 157: The method according to Clause 132, wherein said inlet of said filter device and said outlet of said filter device are removable.
Clause 158: The method according to Clause 132, wherein said pump comprises a portion of medical device selected from the group of medical devices consisting of an apheresis device, a plasmapheresis device, an extracorporeal blood purification device, a hemofiltration device, a continuous veno-venous hemofiltration device, a hemodiafiltration device, an ultrafiltration device, and a hemodialysis or dialysis device.
Clause 159: The method according to Clause 132, wherein said filter comprises a portion of a medical device selected from the group of medical devices consisting of an apheresis device, a plasmapheresis device, an extracorporeal blood purification device, a hemofiltration device, a continuous veno-venous hemofiltration device, a hemodiafiltration device, an ultrafiltration device, and a hemodialysis or dialysis device.

Having thus described the subject matter, it should be apparent that numerous alternative embodiments, modifications, adaptations, and alternative methods may be resorted to without departing from the scope and fair meaning of the subject matter as set forth hereinabove.

1. A filter device for separating and removing undesired matter from blood or a component of blood comprising:
a chamber comprising a top side, a bottom side, and a lateral side;
said chamber comprising a cavity;
an outside height dimension between said top side and said bottom side of said chamber, said chamber comprising a maximum width dimension, said outside height dimension being equal to or greater than said maximum width dimension;
an inlet disposed on said top side and an outlet disposed on said bottom side;
a filter disposed in said cavity;
said filter comprising at least one of a mesh, a screen, and a filter media which is capable of capturing, trapping, or adsorbing said undesired matter; whereby said blood or component of blood can be filtered in said chamber between said inlet and said outlet.

2. The filter device according to claim 1, wherein said at least one of the mesh, the screen, and the filter media comprise a border.

3. The filter device according to claim 1, said chamber comprising a central axis, said at least one of the mesh, the screen, and the filter media comprising a layer, said chamber comprising a plurality of layers disposed on parallel planes which are orientated perpendicular to said central axis.

4. The filter device according to claim 1, wherein said at least one of the mesh, the screen, and the filter media comprise a convoluted configuration.

5. The filter device according to claim 3, wherein said plurality of layers comprise a nested configuration.

6. The filter device according to claim 1, comprising a magnet device disposed proximate to said filter.

7. The filter device according to claim 6, comprising a support structure for removably securing said magnet device.

8. The filter device according to claim 1, wherein said lateral side comprises a curved shape, and said chamber comprises a cylindrical shape.

9. The filter device according to claim 1, wherein said filter media comprises a plurality of magnetic beads.

10. The filter device according to claim 1, wherein said filter comprises a mesh or screen pore size smaller than 30 microns.

11. The filter device according to claim 1, said chamber further comprising a central axis, and a perforated tube disposed about said central axis, said perforated tube being in fluid communication with said outlet, said inlet being configured to be aligned with said outlet and about said central axis, said filter comprising said at least one of the mesh, the screen, and the filter media comprising a plurality of layers of said least one of said mesh, said screen, and said filter media, said plurality of layers being disposed concentrically about said central axis.

12. The filter device according to claim 1, wherein said filter is configured to comprise an electrical charge.

13. A filter device for separating and removing undesired matter from blood or a component of blood comprising:
a chamber comprising a top side, a bottom side, and a lateral side;
said chamber comprising a cavity;
an outside height dimension between said top side and said bottom side of said chamber;
said chamber comprising a maximum width dimension, said outside height dimension being equal to or greater than said maximum width dimension;
an inlet disposed on said top side and an outlet disposed on said bottom side;
a filter disposed in said cavity;
said filter comprising at least one of a mesh, a screen, and a filter media which is capable of capturing, trapping, or adsorbing said undesired matter;
said chamber comprising cylindrical shape and said lateral wall comprising a curved shape;
said chamber comprising a first wall comprising a first outside surface and a first inside surface and comprising a first radius;
said chamber comprising a second wall comprising a second outside surface and a second inside surface and comprising a second radius;
said second wall being concentric to said first wall and said second radius being less than said first radius;
said second wall defining a first space in said cavity of said chamber between said first inside surface of said first wall and said second outside surface of said second wall;
said second wall defining a second space in said cavity of said chamber between opposite portions of said second inside wall;
said filter disposed in said cavity of said chamber in said second space; and, a magnetic device disposed in said cavity of said chamber in said first space, whereby said magnetic device can impart an electromagnetic field upon said filter and said undesired matter contained in said blood or component of blood in said chamber and said blood or component of blood can be filtered in said chamber between said inlet and said outlet.

14. The filter device of claim 1, further comprising a spiral channel disposed in said chamber, said spiral channel descending from proximate to said top side to proximate to said bottom side, said filter disposed within said spiral channel, whereby the length of the flow path of said blood or blood component is greater than said outside height dimension.

15. The filter device of claim 1, further comprising a plurality of baffles disposed in said chamber, said plurality of baffles defining a plurality of channels and openings; each of said plurality of channels being in fluid communication with each other of said plurality of channels through said openings; one or more of said filter being disposed within said plurality of channels; whereby the length of the flow path of said blood or blood component is greater than said outside height dimension.

16. The filter device according to claim 1, wherein said undesired matter comprises at least one of a metal, graphene oxide, graphene hydroxide, magnetic graphene oxide, an electronic chip or other electronic device, a pathogen, and a toxin.

17. A method of treating an individual having undesired matter dispersed in their blood, the method comprising the steps of: inserting a first needle into a first vein or artery of said individual at a first puncture site on said individual and connecting said first needle to at least one piece of tubing, an air bubble eliminator, a stop valve, a filter device comprising at least one of a mesh, screen, and a filter media which is capable of capturing, trapping, or adsorbing said undesired matter, and to a second needle; inserting the second needle into a second vein or artery of the individual at a second puncture site; and manipulating the stop valve to initiate the flow of blood from said first puncture site through the filter device and reinfusing filtered blood through the second puncture site.

18. The method according to claim 17, further comprising applying an electromagnetic field causing said undesired matter to be separated from said blood and be captured, trapped, or adsorbed in said filter device.

19. The method according to claim 17, said filter device comprising a plurality of magnetic beads, whereby said undesired matter is separated from said blood and captured, trapped, or adsorbed in said filter device.

20. The method according to claim 17, and further comprising utilizing at least one of a pump, an analyte sensor, a blood dispersion device comprising a window, an optical device, a blood or blood component conditioning device, and a magnet device disposed between said first needle and said second needle, and monitoring at least one of said pump, said analyte sensor, said blood dispersion device comprising said window, said optical device, said blood or blood component conditioning device, and said magnet device.
